(19) United States
(12) Patent Application Publication
(10) Pub. No.: US 2014/0358140 A1
(43) Pub. Date: Dec. 4, 2014

(54) MICROWAVE TREATMENT DEVICES AND METHODS

(71) Applicant: MicroCube, LLC, Fremont, CA (US)

(72) Inventors: Clarence EMMONS, Capitola, CA (US); Chun Yiu CHU, Oakland, CA (US); Amrish J. WALKE, Milpitas, CA (US); Dinesh I. MODY, San Jose, CA (US); Ketan SHROFF, Pleasanton, CA (US)

(73) Assignee: MICROCUBE, LLC, Fremont, CA (US)

(21) Appl. No.: 14/462,434

(22) Filed: Aug. 18, 2014

Related U.S. Application Data
(63) Continuation-in-part of application No. 12/603,134, filed on Oct. 21, 2009, now Pat. No. 8,808,281.
Application No. 12/603,134 is a continuation-in-part of application No. 12/603,349, filed on Oct. 21, 2009.
Application No. 12/603,349 is a continuation-in-part of application No. 12/603,077, filed on Oct. 21, 2009.
Application No. 12/603,077 is a continuation-in-part of application No. 13/854,673, filed on Apr. 1, 2013.
(60) Provisional application No. 61/222,409, filed on Jul. 1, 2009.
(60) Provisional application No. 61/180,133, filed on May 21, 2009.
(60) Provisional application No. 61/162,241, filed on Mar. 20, 2009.
(60) Provisional application No. 61/107,252, filed on Oct. 21, 2008.
(60) Provisional application No. 61/113,189, filed on Nov. 10, 2008.
(60) Provisional application No. 61/113,192, filed on Nov. 10, 2008.
(60) Provisional application No. 61/113,194, filed on Nov. 10, 2008.
(60) Provisional application No. 61/162,241, filed on Mar. 20, 2009.
(60) Provisional application No. 61/162,244, filed on Mar. 20, 2009.
(60) Provisional application No. 61/222,409, filed on Jul. 1, 2009.
(60) Provisional application No. 61/222,409, filed on Jul. 1, 2009.
(60) Provisional application No. 61/180,133, filed on May 21, 2009.
(60) Provisional application No. 61/162,241, filed on Mar. 20, 2009.
(60) Provisional application No. 61/107,252, filed on Oct. 21, 2008.
(60) Provisional application No. 61/686,125, filed on Mar. 31, 2012.
(60) Provisional application No. 61/752,351, filed on Jan. 14, 2013.

(51) Int. Cl. A61B 18/18

(52) U.S. Cl. CPC A61B 18/1815; A61B 2018/1861; A61B 2018/00005
USPC 606/33

(57) ABSTRACT

The present invention discloses embodiments of microwave antennas and other energy delivery systems. The present invention also discloses systems and methods for the delivery of various medical components within or on a body for performing one or more medical procedures. Several embodiments herein disclose medical systems comprising a combination of one or more medical components and one or more elongate steerable or non-steerable arms that are adapted to mechanically manipulate the one or more medical components. Several embodiments of microwave antennas are disclosed that comprise an additional diagnostic or therapeutic modality located on or in the vicinity of the microwave antennas.

### CROSS-REFERENCE TO RELATED APPLICATIONS

This application is a continuation in part of U.S. patent application Ser. No. 12/603,134, filed Oct. 21, 2009 which claims benefit of U.S. Provisional Patent Application Nos. 61/222,409, filed Jul. 1, 2009; 61/180,133, filed May 21, 2009; 61/162,241, filed Mar. 20, 2009; and 61/107,252, filed Oct. 21, 2008; this application is also a continuation in part of U.S. patent application Ser. No. 12/603,349, filed Oct. 21, 2009, which claims benefit of U.S. Provisional Patent Application Nos. 61/113,189, filed Nov. 10, 2008; 61/113,192, filed Nov. 10, 2008; 61/113,194, filed Nov. 10, 2008; 61/162,241, filed Mar. 20, 2009; 61/162,244, filed Mar. 20, 2009; and 61/222,409, filed Jul. 1, 2009; this application is also a continuation in part of U.S. patent application Ser. No. 12/603,077, filed Oct. 21, 2009, which claims benefit of U.S. Provisional Patent Application Nos. 61/222,409, filed Jul. 1, 2009; 61/180,133, filed May 21, 2009; 61/162,241, filed Mar. 20, 2009; and 61/107,252, filed Oct. 21, 2008; this application is also a continuation in part of U.S. patent application Ser. No. 13/854,673, filed Apr. 1, 2013, which claims benefit of U.S. Provisional Patent Application Nos. 61/686,125, filed Mar. 31, 2012 and 61/752,351, filed Jan. 14, 2013, the contents of each of which is incorporated herein by reference in its entirety.

### FIELD OF THE INVENTION

This invention relates to treatment devices and delivery systems suitable for performing energy based medical procedures within a patient's body.

### BACKGROUND OF THE INVENTION

Microwave antennas have been designed to treat various medical conditions by microwave energy delivery. Such microwave antennas may be used for ablating or otherwise modifying tissue. For example, microwave antennas have been used for ablating cardiac tissue to treat cardiac arrhythmias. In several applications of microwave antennas including cardiac ablations, it is very advantageous to have an additional modality located on the microwave antenna. For example, it is very advantageous to have one or more electrophysiological mapping electrodes located on or near a microwave antenna used for cardiac ablation. Such a design enables the user to ablate cardiac tissue using the microwave antenna and also detect electrophysiological signals using the mapping electrodes. Simply adding an additional modality such as mapping electrodes and the conductive wires over a microwave antenna may create a microwave antenna that is unsuitable for clinical use. The additional modality and the conductive wires may absorb microwave energy and become hot. This in turn may causes problems such as burning or charring of tissue, creation of undesirable blood clots and adherence of the microwave antenna to tissue. In addition, additional modality and the conductive wires connected to the additional modality may change the shape of the microwave field emitted by the microwave antenna. This in turn may create a shaped microwave field that is no longer clinically useful. Such a shaped microwave field may also create additional safety issues by undesired microwave energy delivery to healthy tissue. The microwave field may also affect the functioning of the additional modality. Thus, there is a need for microwave antennas that comprise such additional modality such that the safety and the performance of such antennas is not compromised.

Medical diagnostic and surgical procedures generally require that physicians perform many increasingly complex functions within the body. In many of these procedures, the physicians must properly access tissue locations and precisely orient a medical component to perform a diagnostic or surgical procedure. In an ever increasing number of modern surgical procedures, elongate devices are introduced into the target tissue through surgically created access openings or natural openings in the body. Physicians deliberately keep such access openings small to minimize the trauma and the healing burden on the patient. For example, in many catheter-based cardiac procedures, a physician will access internal cardiac tissue by navigating a device through the vasculature until the device can engage the cardiac tissue (e.g. a chamber of the heart). In another example, a physician may introduce an ablation catheter into the renal arteries for performing a renal denervation procedure. In another example, a physician may create an opening (or rely upon a natural body opening) to access organs and tissues requiring a medical procedure. Traditionally, the desire to minimize trauma to the patient often comes at the expense of the maneuverability of the medical component when compared to a similar open surgical procedure. For instance, a physician working with his hands and having full access to a target site such as during an open surgery has much greater freedom to manipulate one or more medical devices to access target tissue and position and orient the medical devices as needed to perform many functions. Yet, such freedom is lost during minimally invasive procedures and in some open surgical procedures where the physician's access to the target site is limited due to obstructing tissue or organs (such as accessing the interior of an organ through a wall of the organ).

Many conventional approaches attempt to overcome these limitations on maneuverability by providing a device with a steerable distal end. However, these existing devices are unable to provide the optimal amount of articulation and/or manipulation needed within the body, especially when the procedure is performed under fluoroscopy.

To illustrate this point, consider the situation in an open surgical procedure, where a physician is able to use one or both arms to directly manipulate a treatment device onto or along a body structure. The numerous degrees of freedom offered by a single arm (including the hand, the wrist and fingers) enables satisfactory placement of the treatment device. When the physician attempts the same procedure through a limited access (either a smaller incision, use of access ports, through the vasculature or even through dissection of other tissues/organs), conventional steerable devices do not provide sufficient maneuverability. For example, catheter-based interventional endocardiac ablations for treating Atrial Fibrillation suffer from shortcomings due to a lack of access to all target regions within the atria. For a successful procedure, it is critical to access specific target regions within the left atrium and create a series of ablations in a specific pattern. Existing steering devices are inadequate in providing this function. This reduces the efficacy of the procedure, increases the procedure times and necessitates the use of very expensive accessory devices such as surgical navigation systems.

Further, there is a need for devices and methods that can deliver therapeutic energy to vascular regions such as renal artery walls for procedures such as renal denervation to treat conditions such as hypertension. Such devices and methods need to deliver a controlled amount of energy accurately to the desired target vascular region while minimizing any collateral damage to non-target tissue.

In view of the above, there remains a need for a device or system that offers improved positioning of one or more medical device components in various medical procedures. There also remains a need for a device or system that provides controlled energy delivery to target anatomical locations. There also remains a need for a device or system that allows a physician the capability to position an appropriate medical component readily and predictably at all desired locations (where the locations may be 2 or 3 dimensional structures) for performing the required procedures such as catheter based cardiac ablation, renal denervation, etc.

### SUMMARY OF THE INVENTION

The systems and methods described herein are useful in a variety of medical procedures including various diagnostic and therapeutic procedures. In addition, the invention is also useful in open surgical procedures, minimally invasive procedures, as well as procedures performed through natural body openings such as NOTES procedures.

In a significant amount of the disclosure, the heart is used an example of a target organ and cardiac ablation procedures are used as an example of procedures that may be performed using the current invention. However, it should be noted that the various methods and devices disclosed herein may also be used in medical procedures ranging from endovascular cardiac, thoracic cardiac, bronchial (e.g. bronchial thermoplasty), lung, neurological, gynecological, gastro-intestinal, spinal, ENT, laparoscopic, arthroscopic and other endoscopic procedures, robotic including tele-robotic, oncological, etc. Specifically, the various methods and devices disclosed herein may be used to ablate one or more regions of the renal artery wall and its surrounding anatomy to treat hypertension and other conditions.

Some of the embodiments herein may be broadly described as microwave devices comprising a transmission line such as a coaxial cable and an antenna connected to the coaxial cable. Further, an additional diagnostic or treatment modality is located adjacent to or over the antenna. The antenna comprises 1. a radiating element, 2. one or more shaping elements and 3. one or more antenna dielectrics covering one or more portions of the radiating element and/or the shaping element. In embodiments wherein transmission line is a coaxial cable, the radiating element may be a continuation of the inner conductor of the coaxial cable or may be an additional conductive element electrically connected to the inner conductor of the coaxial cable. The radiating element radiates a microwave field that is characteristic of its specific design. The radiated microwave field causes agitation of polarized molecules, such as water molecules, that are within target tissue. This agitation of polarized molecules generates frictional heat, which in turn raises the temperature of the target tissue. Further, the microwave field radiated by the radiating element may be shaped or otherwise redistributed by one or more shaping element(s) in the antenna. In one embodiment, the shaping element(s) are made of an electrically conductive material (e.g. one or more metallic objects of various sizes, shapes, orientations, etc.). In this embodiment, the shaping element(s) may be electrically connected to the outer conductor or shielding element of the transmission line (e.g. the outer conductor of a coaxial cable). In an alternate embodiment, the shaping element(s) are not in direct electrical conduction with the outer conductor or shielding element of the transmission line e.g. the outer conductor of a coaxial cable. The one or more antenna dielectrics may cover one or more portions of one or both of: radiating element and shaping element. The antenna dielectrics may be used for changing the propagation of the microwave field from one or both of: radiating element and shaping element to the surrounding. The antenna dielectrics may be used for changing the matching of the antenna.

The present invention discloses multiple embodiments of planar, linear and three dimensional microwave antennas. Such antennas may be flexible to allow introduction of the antenna into a bodily region in a collapsed configuration through an opening of a small size. The antennas disclosed herein may adapt to bodily regions of different sizes and shapes to allow the user to perform the desired action regardless of the size and shape of the target anatomy.

The microwave devices disclosed herein may use a variety of feedback mechanisms during their use. In one such embodiment, one or more returned power measurements are used to take a variety of decisions before, during, or after an energy delivery procedure.

One or more diagnostic or treatment modalities may be a part of the microwave device or may be located on an additional device that is located around the microwave device. One or more conductive wires may be connected to the diagnostic or treatment modalities that connect the diagnostic or treatment modalities to an external circuit. The diagnostic or treatment modalities and the conductive wires connected to the diagnostic or treatment modalities are located within the microwave field emitted by the antenna. The novel configuration and placement of such diagnostic or treatment modalities and their conductive wires ensures that the temperature of the additional modality and the conductive wires does not exceed a safe level during clinical use. Further, the presence of the additional modalities and the conductive wires does not affect or minimally affects the shape of the microwave field emitted by the microwave antenna. This in turn ensures that the safety and the performance of such antennas are not compromised.

Various additional features may be added to the devices disclosed herein to confer additional properties to the devices disclosed herein. Examples of such features include, but are not limited to one or more lumens, ability to apply a vacuum or suction to the target anatomy, ability to visualize one or more regions of the target anatomy, ability to limit the depth of insertion into the target anatomy, ability to deploy the antenna, ability to connect to a source of energy, etc.

The dimensions or other working parameters of the devices disclosed herein may be adjustable or programmable based on user inputs. The user input may be based on factors such as patient's anatomical data including anatomical dimensions and the desired level of safety and efficacy.

The various microwave antennas and the microwave engineering principles disclosed herein may be also used in a variety of non-medical applications. The near field of the microwave antennas disclosed herein may be used on target materials such as food, industrial products, semiconductors, etc. The near field of the microwave antennas disclosed herein may be used for cooking or heating foods, in industrial processes for drying and curing products, in semiconductor processing techniques to generate plasma for processes such as reactive ion etching and plasma-enhanced chemical vapor deposition (PECVD).

The device and methods disclosed herein may be used with or without modifications to create one or more point, linear, area or volumetric lesions. The present invention discloses various embodiments of flexible, low-profile devices that can be inserted non-invasively or minimally invasively into or near the target tissue.

The present invention also comprises medical systems for steering medical components or devices in the anatomy. In one embodiment, the medical system comprises at least a first and second arm, each arm having a maneuverable distal portion and a proximal portion, where manipulation of the respective arm's proximal portion permits articulation of the arm's maneuverable distal portion independently of the other respective arm, and a medical component coupled to the first arm and extendable from the first arm's maneuverable distal portion, the medical component being engageable with the second arm's maneuverable distal portion, such that when coupled to both arms, movement of either maneuverable distal portion alters a profile or position of the medical component allowing for positioning of the medical component to perform the medical procedure.

In another embodiment, the invention includes a medical system for performing a medical procedure on or in a patient, the system comprising a first arm having a maneuverable distal portion and a proximal portion, wherein manipulation of the arm's proximal portion permits articulation of the arm's maneuverable distal portion and a medical component coupled to the first arm and extendable from the first arm's maneuverable distal portion. Movement of the maneuverable distal portion alters a profile or position of the medical component allowing for positioning of the medical component to perform the medical procedure.

In some variations, the medical components extend through an arm in a concentric or coaxial manner. However, the invention also includes components that extend parallel to the arm, or that extend along the arm or a portion thereof in a non-concentric manner. One or more arms may be removably coupled to the medical component(s) via a grasping structure. An example of such grasping structure includes a releasable hook, ring, or jaws. The arms can fully or partially hollow. One or more arms may be concentric with each another. One or more arm may be introduced via one or more access ports and pathways.

The term medical component is intended to include a medical device or portion thereof that is adapted to provide a visual, diagnostic, or treatment procedure. The medical system can include a first medical component comprising an energy delivery device configured to deliver energy (e.g. microwave, radiofrequency, DC, ultrasound, laser, a cryogenic field) to or from tissue and a second medical component (such as a guidewire, rail, tether, etc.) that is used to direct the energy delivery device towards a target site. The first and second medical component can be parts of the same device or can be separate medical devices.

Examples of medical components, include, but are not limited to, therapeutic devices such as ablation devices for imparting a treatment relative to a target tissue, diagnostic devices such as mapping catheters for providing physiological information regarding a target tissue; positioning devices which include elements for providing additional positioning of additional functional devices (e.g., guidewires, rails, tethers, introducer catheters, sheaths, etc.), imaging devices, or non-imaging feedback devices (such as a Doppler catheter). The medical component need not have a specific physical structure, for example the arms of the inventive system can be adapted to deploy a simple tube that administers a chemical ablating agent at a desired location or deploy an additional fluid used during, and in support of, the medical procedure, for example deployment of contrast agent to provide a clearer view of the anatomy in support of a procedure performed within a patient's heart. In yet additional variations, the medical components can include separate components used to provide a single diagnostic procedure or medical of the same medical procedure. For instance, when using a radiofrequency energy modality, the medical component could include a first electrode while the second component can include a second electrode (either the opposite or same polarity).

The tether member may consist of a string or wire like structure that is used to simply pull the medical component through one or both arms. In additional variations, the tether member can include a flexible tether member that, when deflected, assumes a curvilinear shape based on the structural characteristics of the tether. As discussed below, this allows the medical component to assume a “U” shaped configuration that can assist in performing the medical procedure. The tether member may also comprise one or more conductive wires connected to one or more diagnostic or therapeutic modalities. In one embodiment, the one or more conductive wires are a part of the tether. In one embodiment, the one or more conductive wires extend along the tether and are attached to the tether at one or more regions. In one embodiment, the one or more conductive wires extend along the tether and are unattached to the tether.

Another aspect of the system is the ability to control movement of the distal portions of the arms from the proximal portions. In one embodiment, the arms include handle portions on the proximal end. The handle portions can include one or more steering control mechanisms. The movement of the arms can occur in any three dimensional space.

In an addition embodiment, the devices and method described herein include a medical system for performing a medical procedure on or in a patient, the system comprising a medical component, a first arm having a distal portion and a proximal portion, where manipulation of the first arm's proximal portion permits articulation of the first arm's distal portion and where the medical component is coupled to and advanceable relative to the distal portion of the arm, a second arm having a distal portion and a proximal portion, where manipulation of the second arm's proximal portion allows for articulation of the second arm's distal portion, a rail member or tether coupled to the medical component and extending through the second arm, and where the first and second arms are configured to be manipulated independently, and where manipulation of the first or second arm alters a profile or position of the medical component allowing for positioning of the medical component.

Also disclosed are variety of lesion patterns such as point lesions, linear and non-linear lesions, area lesions and volumetric lesions and combinations thereof created on the target tissue using the methods and devices disclosed herein.

### BRIEF DESCRIPTION OF THE DRAWINGS

FIG. 1A shows a side view of an embodiment of a linear microwave antenna of the present invention comprising a radiating element and a microwave field shaping element.

FIG. 1B shows a side view of an embodiment of a microwave antenna comprising one or more functional elements such as electrophysiological mapping or pacing or RF ablation electrodes.

FIG. 1C shows a section through an embodiment of a coaxial cable usable for the ablation devices of FIGS. 1A, 1B other ablation devices disclosed herein.

FIG. 1D shows a longitudinal section of the embodiment of the ablation device of FIG. 1A through the distal end of the coaxial cable.

FIGS. 1E-1G show the method steps of a method of creating two overlapping lesions in a tissue.

FIG. 1H shows a side view of an embodiment of a microwave antenna comprising multiple electrophysiological mapping or pacing electrodes that are spaced in regular intervals along the length of the antenna.

FIG. 1I shows a side view of an embodiment of a microwave antenna comprising multiple electrophysiological mapping or pacing electrodes that are spaced in pairs along the length of the antenna.

FIG. 1J shows a side view of a functional microwave antenna with a design similar to the design shown in FIG. 1A.

FIG. 1K shows a side view of a functional microwave antenna having additional electrodes.

FIGS. 1L and 1M show surface images of lesions obtained by ablating the surface of porcine muscle tissue using the antennas shown in FIGS. 1J and 1K respectively.

FIGS. 1N and 1O show sections of the porcine tissue in FIGS. 1L and 1M showing the depth of the lesions obtained by the antennas in FIGS. 1J and 1K respectively.

FIG. 1P shows a side view of a simulated SAR profile generated by an antenna similar to the antenna of FIG. 1A.

FIG. 1Q shows a side view of a simulated SAR profile generated by an antenna similar to the antenna of FIG. 1A but having four extra electrodes spaced at regular intervals along the length of the antenna wherein each electrode is connected to a conductive wire arranged in a first configuration.

FIG. 1R shows a side view of a simulated SAR profile generated by an antenna similar to the antenna of FIG. 1A but having four extra electrodes spaced at regular intervals along the length of the antenna wherein each electrode is connected to a conductive wire arranged in a second configuration.

FIG. 1S shows a view of the distal region of the ablation device similar to the ablation device used in FIGS. 1K, 1M and 1O.

FIG. 1T shows an antenna similar to the antenna in FIG. 1A being slidably introduced through the lumen of a functional tube.

FIG. 1U shows a side view of a simulated SAR profile generated by a 915 MHz monopole antenna.

FIG. 1V shows a side view of a simulated SAR profile generated by a monopole antenna similar to the antenna of FIG. 1U but having four extra electrodes spaced at regular intervals along the length of the antenna wherein each electrode is connected to a conductive wire arranged in a first configuration.

FIG. 1W shows a side view of a simulated SAR profile generated by a monopole antenna similar to the antenna of FIG. 1U but having four extra electrodes spaced at regular intervals along the length of the antenna wherein each electrode is connected to a conductive wire arranged in a second configuration.

FIGS. 2A-2D show various configurations of a flexible linear microwave antenna deployed in target regions of varying shape.

FIG. 2E shows an embodiment of an antenna similar to the antenna in FIGS. 2A-2D comprising a radiating element and a shaping element connected to each other by one or more flexible dielectric attachments.

FIGS. 2F, 2G and 2H show three configurations of the antenna of FIG. 2E in a constrained configuration, a less constrained configuration and a least constrained configuration respectively.

FIG. 2I depicts an ablation system comprising a guide sheath and a medical component comprising a transmission medium or transmission line ending in an ablating portion.

FIG. 2J shows an embodiment of an ablation system comprising a guide sheath that is adapted to approach a target tissue substantially normal to the tissue surface.

FIGS. 2K and 2L show sections through the device of FIG. 2I through sections 2K-2K and 2L-2L respectively.

FIG. 2M shows a method embodiment of creating of three overlapping lesions.

FIG. 2N shows an embodiment wherein an ablation device and a guide sheath are one operative unit.

FIGS. 2O and 2P show an embodiment of a medical system comprising a sheath or arm that is used to introduce and position a medical component.

FIG. 2Q shows an embodiment of a medical system comprising an arm, wherein the distal region of the arm is deflected by about 200-280 degrees.

FIG. 2R shows a medical system comprising a medical component that is partially deployed from the distal end of an arm.

FIG. 2S shows an embodiment of a medical system similar to the medical system in FIG. 2O.

FIG. 2T shows a view of a medical system wherein a medical component is fully extended from a sheath and forms a looped shape.

FIGS. 2U-2X show an embodiment of a method to treat a bodily region using minimally invasive techniques.

FIG. 3A shows an embodiment of an ablation device with a three dimensional antenna comprising a radiating element and multiple shaping elements adapted to ablate a volume of tissue.

FIGS. 3B and 3C show a side view and a top view of a simulated SAR profile of an embodiment of the antenna of FIG. 3A.

FIGS. 3D and 3E show a side view and a top view of a thermal simulation of an embodiment of the antenna of FIG. 3A.

FIGS. 3F and 3G show a side view and a top view of a simulated SAR profile at 0.915 GHz of an embodiment of an antenna similar to the antenna of FIG. 3A.

FIG. 3H shows the simulated return loss of an ablation device with an antenna of FIGS. 3F and 3G.

FIGS. 3I-3K show the steps of using an antenna in three different locations of different dimensions within a single target region.

FIGS. 3L-3N show the steps of a method of delivering microwave energy to a target material wherein the properties of the target material change as the microwave energy is delivered.

FIGS. 3O and 3P show an antenna similar to that in FIG. 3I in a constrained configuration and a less constrained configuration respectively.

FIG. 4A shows a view of a planar antenna of a microwave ablation device designed for endometrial ablation.

FIG. 4B shows a section of the ablation device of FIG. 4A through the distal end of a coaxial cable.

FIG. 4C shows the antenna of FIG. 4A with the center loop removed for clarity.

FIGS. 4D and 4E show the front and side views respectively of the SAR profile generated by an antenna with a center loop similar to the antenna of FIG. 4A.

FIG. 4F shows the simulated return loss of an ablation device with antenna 112 of FIG. 4D.

FIG. 4G shows the front view of an SAR profile generated by the antenna of FIG. 4D without center loop.

FIG. 4H shows the simulated return loss of an ablation device with an antenna of FIG. 4G.

FIG. 5A shows a step of one such embodiment of performing a renal denervation procedure using a linear or curved antenna 112.

FIGS. 5B-5G show various embodiments of lesion patterns that can be made on the wall of a hollow organ or other anatomical region using one or more devices and methods disclosed herein.

FIG. 5H depicts a lesion pattern in the left atrium.

FIGS. 5I-5V show embodiments of flexible non-linear or planar ablating portions or antennas in accordance with the present invention.

FIG. 5W shows an embodiment of an ablating portion comprising three spline members which are each individual antennas.

FIG. 5X shows an embodiment of an ablating portion comprising two concentric curvilinear spline members.

FIGS. 5Y-5AN show embodiments of ablating portions in accordance with the present invention.

### DETAILED DESCRIPTION OF THE INVENTION

The present invention discloses devices and methods for treating tissue with microwave energy or other energy modalities. In several method embodiments, microwave energy is used for ablating tissue e.g. for treating atrial fibrillation by controlled ablation of left atrial tissue, for trating hypertension by renal denervation, etc. The systems and methods described herein are useful in a variety of medical procedures including various diagnostic and surgical procedures. In addition, the invention is also useful in open surgical procedures, minimally invasive procedures, as well as procedures performed through natural body openings.

In a significant amount of the disclosure, the heart or the renal arteries are used as examples of target organs and cardiac ablation and renal denervation procedures are used as examples of procedures that may be performed using the current invention. However, it should be noted that the various methods and devices disclosed herein may also be used in medical procedures including, but not limited to: endovascular cardiac, thoracic cardiac, bronchial, lung, gynecological, gastro-intestinal, spinal, ENT, laproscopic, arthroscopic procedures, etc.

Microwave thermal ablation does not depend on the conduction of electricity to tissue unlike radiofrequency (RF) ablation. Thus, devices using microwave thermal ablation such as some of the devices disclosed herein don't need good contact with tissue. They can function well even without perfect contact with the target tissue. Thus, the devices disclosed herein do not require extremely precise placement in tissue, thereby reducing the dependence of procedure outcome on physician skills. In some embodiments, the devices herein are designed to have a proximal shaft and a distal microwave emitting portion comprising an antenna. The proximal shaft comprises a transmission line such as a flexible coaxial cable that delivers microwave energy from a microwave generator to the microwave emitting portion. The shaft can be designed to be slim (e.g. <3 mm in diameter) to enable the introduction of the ablation device through narrow openings. The shaft can be designed to be flexible such that minimal forces are exerted on bodily tissues during the introduction of the ablation devices into the anatomy. The flexible nature of the shaft enables the shaft to take the natural shape of passage during introduction instead of distorting the passage by the shaft of the device. The designs of the coaxial cables disclosed herein confer sufficient flexibility to the device shaft such that the device shaft is capable of bending by more than 45 degrees when it experiences distorting forces by the anatomy. If desired, the device shaft may be made stiffer by adding one or more coatings, coverings, stylets and other stiffening elements.

Several of the microwave antenna designs and tests disclosed herein were done for the 0.915 GHz or 2.45 GHz ISM bands. Antennas, methods, etc. disclosed herein may be used with or without modifications at other frequencies including, but not limited to ISM bands of 0.433 GHz, 5.8 GHz, etc. The microwave power generator used in this invention may be magnetron based or solid state. The microwave power generator may be single or multi-channel. The microwave power generator used for the experiments comprised a Vector Network Analyzer (Agilent 8753 series) and amplifier modules build in-house using transistors from Freescale Semiconductor (Austin, Tex.). The power measurement was made using a power meter (ML2438A Power Meter, Anritsu Company, Richardson, Tex.). Similar devices and components can be used to design the microwave generator for clinical use with the devices and methods disclosed herein.

In the experiments, where desired, a fiber optic thermometry system (FOT Lab Kit by LumaSense Technologies, Santa Clara, Calif.) was used to measure the temperature at several locations in the tissue. The fiber optic thermometry system was used since it has no metallic components that might interfere with the microwave field. Similar non-interfering thermometry may be used to measure the temperature at one or more locations during an ablation procedure.

Several devices and methods described herein provide a physician with an improved ability to remotely position one or more medical components on or in a target tissue location. The ability to position such medical components allows the physician to perform one or more medical procedures with improved accuracy and efficiency where direct use of the physician's arms is not possible. Although variations of the system described below primarily discuss placement of one or more energy delivery devices on tissue, such variations are for exemplary purposes only. The features of the system can be used to position any number or type of medical components including, but not limited to: implants or implant delivery devices, diagnostic devices, imaging devices, biopsy devices, radiation emitting devices, drug delivery devices, radio-opaque markers, valvular annuloplasty devices, suturing devices, etc. The system could also be used as a stable platform to perform minimally invasive surgical procedures. In such procedures, the devices and methods disclosed herein may be used to cut, suture, coagulate or remove tissue for example. Any suitable imaging modality may be used to visualize the anatomy and/or one or more of the devices disclosed herein while performing any of the procedures disclosed herein. Examples of such imaging modalities include, but are not limited to endoscopes (e.g. colonoscopes, laparoscopes, thoracoscopes, bronchoscopes, cystoscopes, colposcopes, hysteroscopes, arthroscopes, etc.), X-rays, Computed tomography (CT), fluoroscopy, ultrasound imaging including intravascular and intracardiac ultrasound imaging, intra-cardiac electrophysiological three dimensional mapping, MRI, PET, near infra-red imaging, etc. When an endoscopic modality is used for imaging, one or more components of medical system 100 may be inserted through or simultaneously with the endoscopic instrumentation. In one embodiment, components of medical system 100 are inserted independently from endoscopic instrumentation. Any of the radiological methods disclosed herein may be used with or without a positive or negative contrast agent.

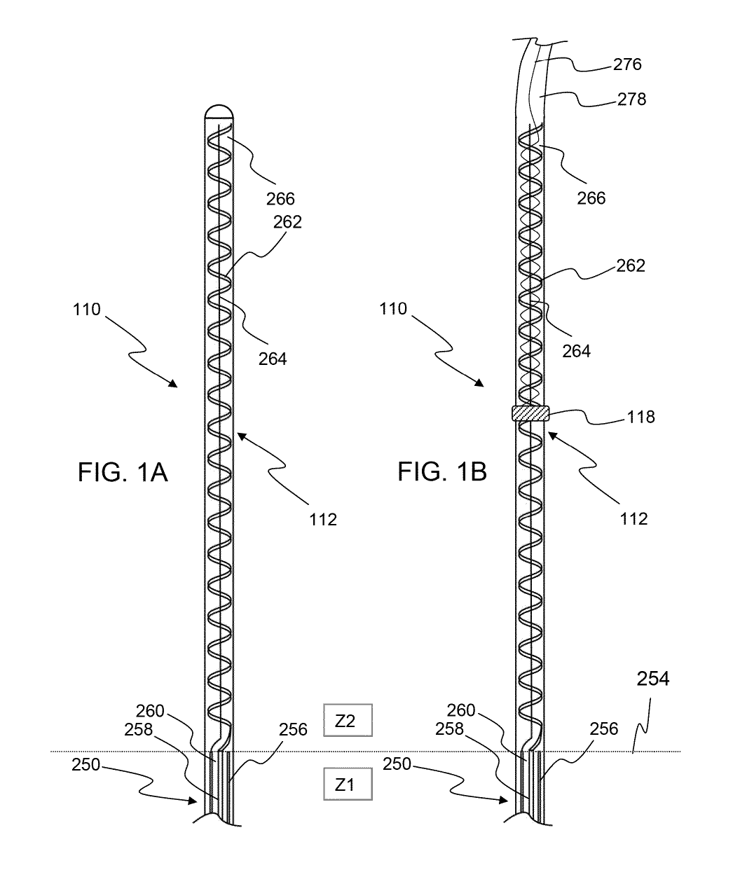

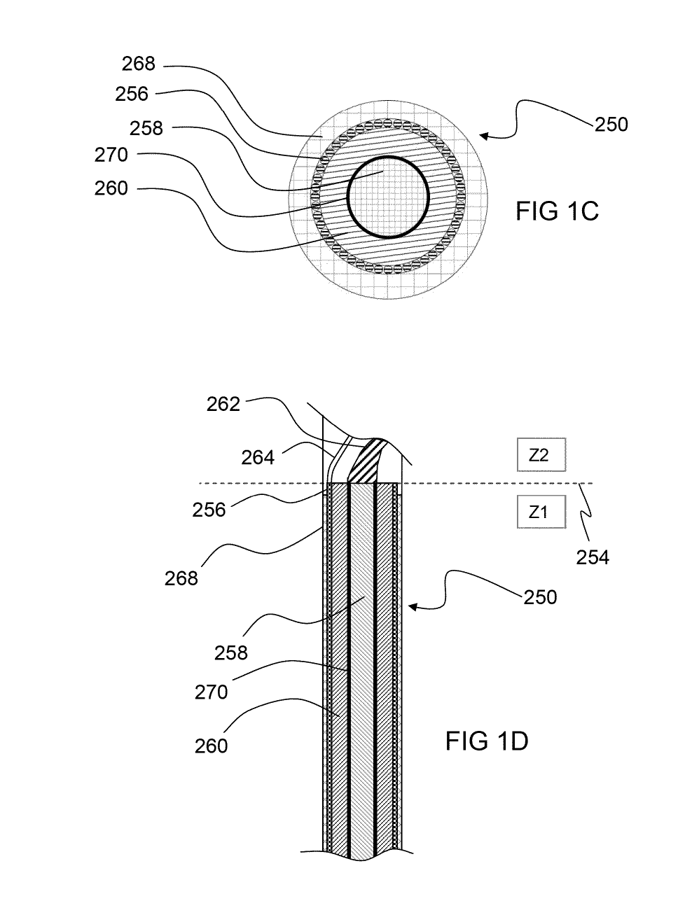

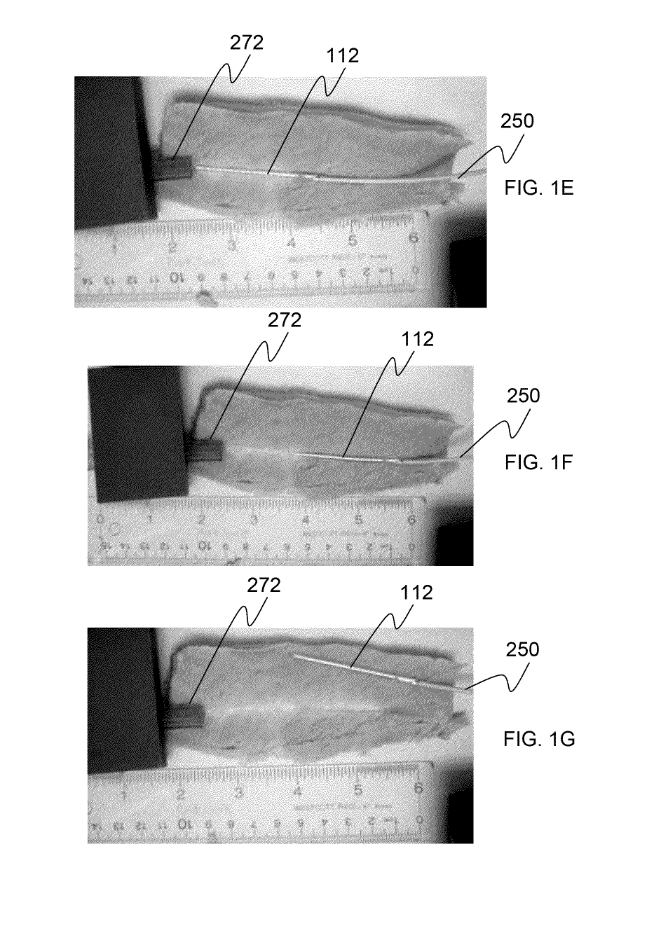

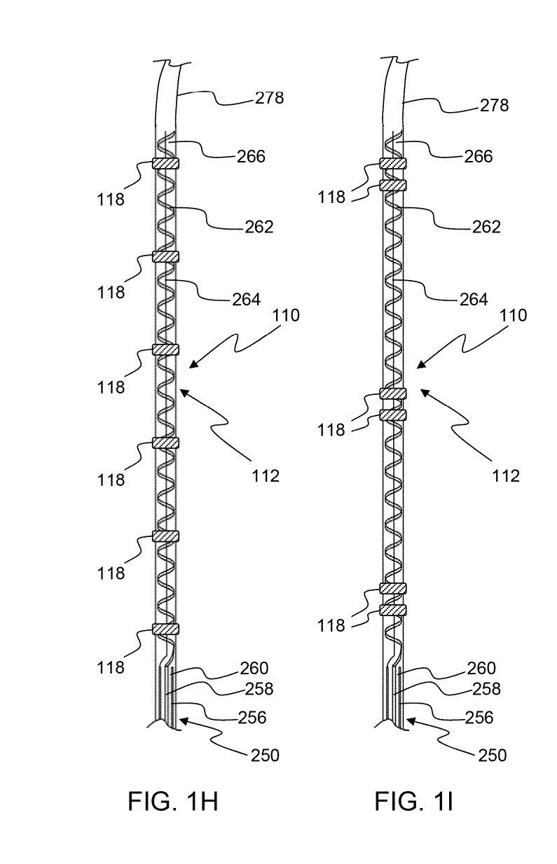

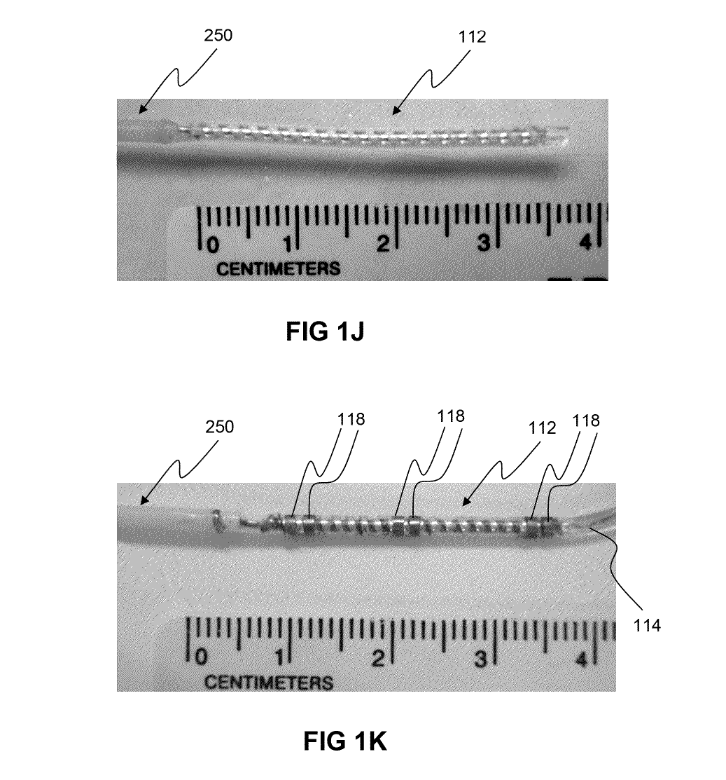

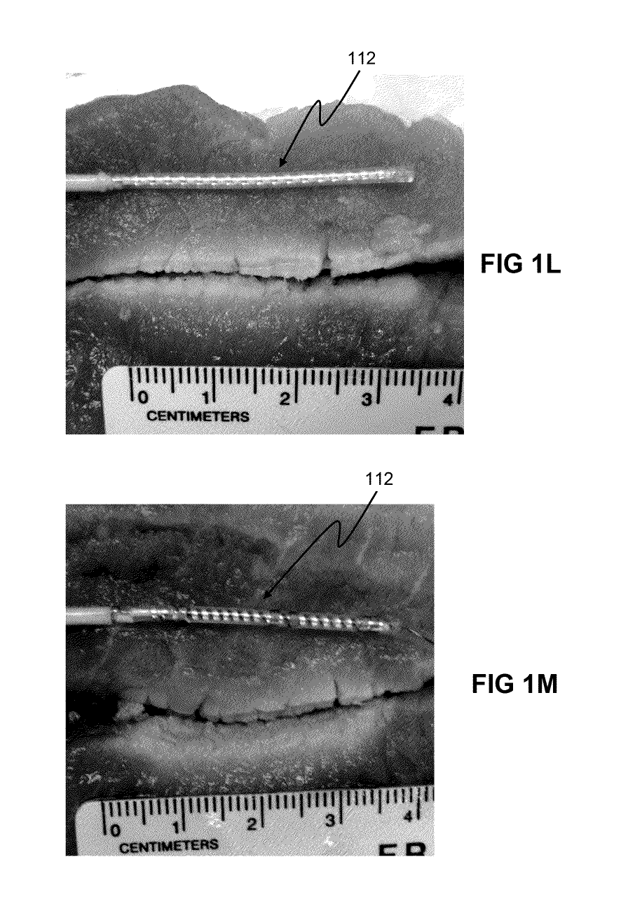

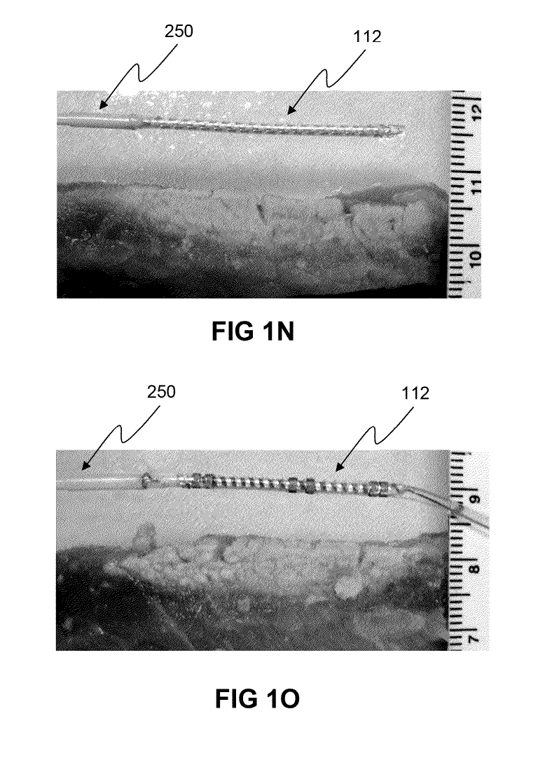

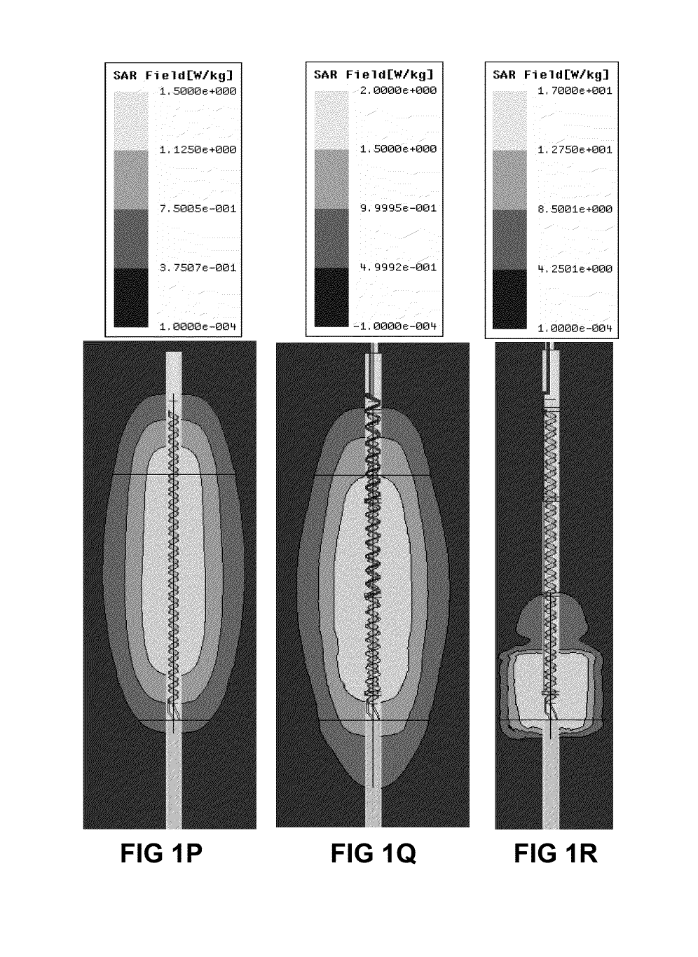

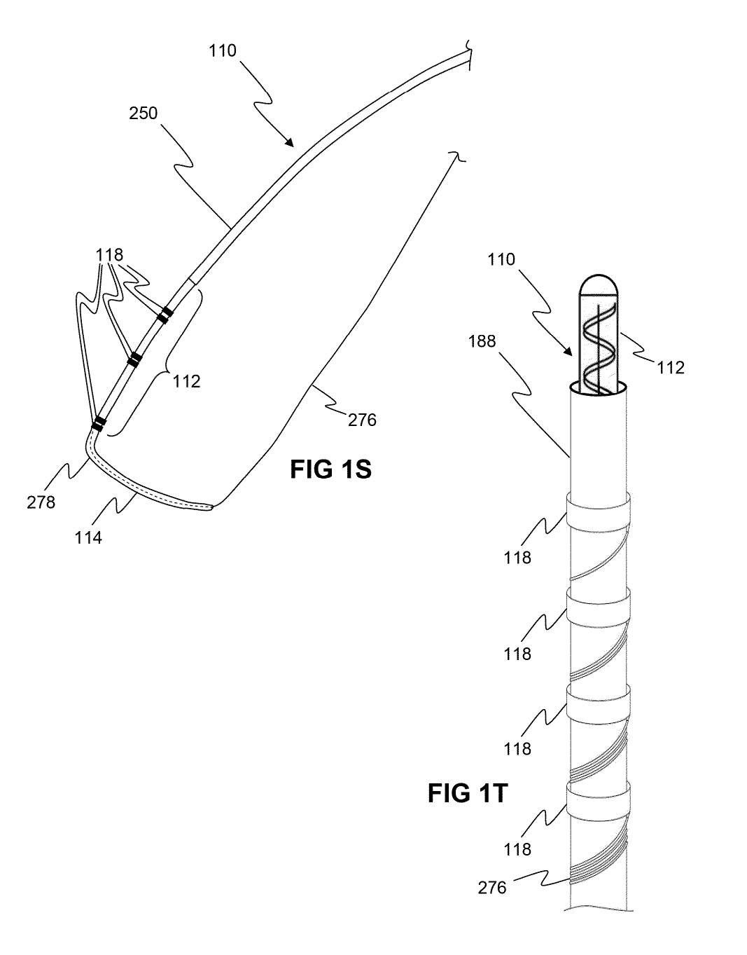

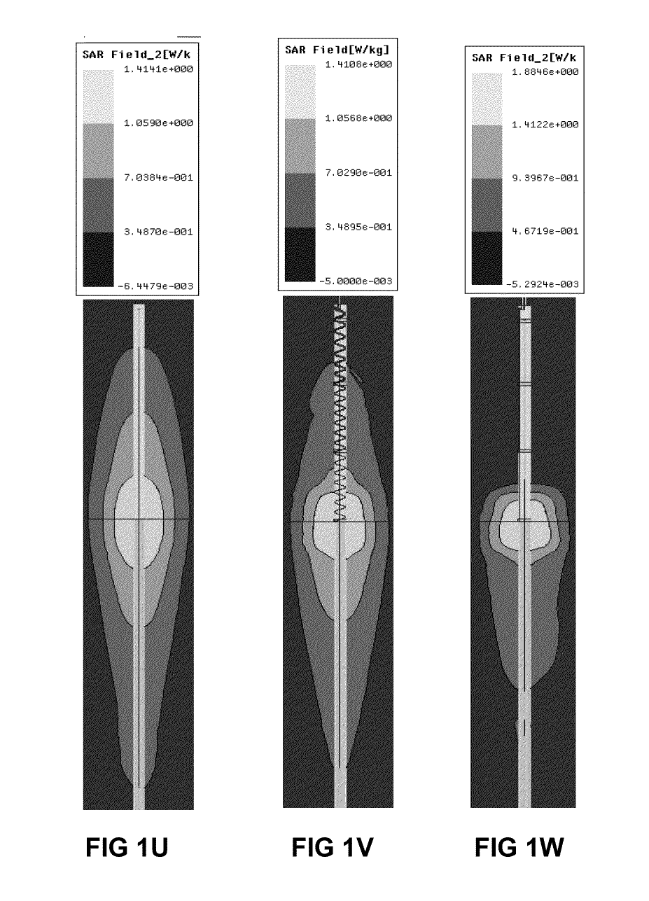

FIG. 1A shows a side view of an embodiment of a linear microwave antenna of the present invention having a microwave antenna comprising a radiating element and a microwave field shaping element. In FIG. 1A, microwave ablation device 110 comprises a transmission line such as a coaxial cable 250. A working element such as an antenna 112 is connected to the distal end of coaxial cable 250. FIG. 1A shows microwave ablation device 110 divided into a first zone Z1 and a second zone Z2 by an imaginary transition line 254. First zone Z1 is proximal to second zone Z2. Transition line 254 is defined by the distal end of coaxial cable 250 and is perpendicular to the axis of coaxial cable 250 at the distal end of coaxial cable 250. In the embodiment shown in FIG. 1A, the distal region of coaxial cable 102 lies entirely within first zone Z1 and antenna 112 lies entirely within second zone Z2. In a one embodiment, a single microwave signal is fed to antenna 112 through coaxial cable 250. Antenna 112 generates a microwave field. The near field of the microwave field generated by antenna 112 may be used for procedures such as tissue ablation.

In FIG. 1A, antenna 112 comprises a radiating element 262 and a shaping element 264. Radiating element 262 may be made of a variety of conducting materials e.g. metals, conductive polymers, materials with embedded conductive particles, etc. When microwave energy is delivered through coaxial cable 250 to antenna 112, a first microwave field is emitted by radiating element 262. The first microwave field interacts or couples with shaping element 264. This interaction induces a leakage current on shaping element 264. The leakage current in turn creates a second microwave field. The first microwave field and the second microwave field together combine to produce a unique shaped microwave field of antenna 112 that is clinically more useful that the unshaped microwave field generated by an antenna 112 comprising only radiating element 262. Thus the original microwave field is redistributed by the design of shaping element 264. Shaping element 264 alone is not capable of functioning as an antenna; rather shaping element 264 shapes or redistributes the electromagnetic or microwave field emitted by radiating element 262 to produce a clinically improved microwave field. It should be noted that there is no direct electrical conduction between radiating element 262 and shaping element 264. Antenna 112 further comprises one or more antenna dielectrics 266 covering one or more portions of one or both of: radiating element 262 and shaping element 264. In FIG. 1A, an antenna dielectric 266 covers the entire radiating element 262. Any of the antenna dielectrics 266 disclosed herein may be used to shape the microwave field and to optimize the performance of antenna 112. Any of the antenna dielectrics 266 disclosed herein may be replaced by one or more conducting polymers.

A microwave field couples to the nearest conductive path. In prior art monopole antennas, the nearest conductive path is provided by the shielding element of the transmission line (e.g. the outer conductor 256 of the feeding coaxial cable 250). This causes a strong concentration of the microwave field in the junction between antenna 112 and transmission line 250. However, in several embodiments of antenna 112 disclosed herein, the nearest conductive path is provided by shaping element 264. Thus the microwave field couples to shaping element 264 instead of coupling to the shielding element of the transmission line (e.g. the outer conductor 256 of the feeding coaxial cable 250). Therefore, minimal microwave field is coupled proximally to the shielding element of the transmission line. This in turn creates a unique, shaped or redistributed microwave field that does not significantly extend proximally to antenna 112 as shown in FIGS. 1P and 1Q. Further, the combination of radiating element 262 and shaping element 264 improves the power deposition of antenna 112.

Antennas disclosed herein may comprise one or more shaping elements 264 made of a variety of conducting materials e.g. metals, conductive polymers, materials with embedded conductive particles, etc. Such shaping elements 264 may comprise one or more dielectrics layers to insulate the shaping element 264 from surrounding tissue. Examples of such shaping elements 264 include, but are not limited to: straight or curved segments of metallic elements, elements with a circular or oval shape, elements with a polygonal shape (e.g. triangular, square, rectangular, pentagonal, etc.), multiple elements joined together by one or more electrically conducting joint(s), multiple elements joined together by a non-electrically conducting joint(s), elements with multiple curves, symmetrically arranged segments of elements, non-symmetrically arranged segments of elements, etc.

In one embodiment, radiating element 262 is a continuation of the inner conductor 258 of a coaxial cable 250. In a one embodiment, shaping element 264 is made of an electrically conductive material e.g. a metal and is electrically connected to a region of outer conductor 256 of coaxial cable 250. In an alternate embodiment, antenna 112 comprises one or more conductive shaping elements 264 that are electrically isolated from outer conductor 256. In this embodiment, one or more shaping elements 264 function as passive radiators or parasitic elements of antenna 112. In one embodiment, shaping element 264 is designed to act as a microwave shielding element and/or a microwave reflecting element.

Further, antenna 112 may be designed to be sufficiently flexible such that during and after introduction and deployment of antenna 112 in the anatomy, the anatomy experiences only slight forces from antenna 112. This may be achieved by designing an antenna 112 comprising one or more flexible radiating elements 262, one or more flexible shaping elements 264 and one or more flexible antenna dielectric materials. Sufficiently flexible antennas may reduce damage to healthy tissue as well as potentially reduce the pain experienced by the patient during the introduction and deployment. Antenna 112 may be introduced through a small lumen. This enables the introduction of antenna 112 through narrow catheters, shafts, introducers and other introducing devices. Further, this enables the introduction of antenna 112 through small natural or artificially created openings in the body. Further, antenna 112 may be designed to have an atraumatic distal end to reduce the risk of perforation of tissue. The flexible nature of antenna 112 enables antenna 112 to take the natural shape of an introduction passage during introduction instead of distorting the passage. In one embodiment, the length of straightened radiating element 262 measured along the radiating element 262 from the distal end of coaxial cable 250 or other transmission line until the distal end of radiating element 262 is an odd multiple of one quarter of the effective wavelength at one of: 433 MHz ISM band, 915 MHz ISM band, 2.45 GHz ISM band and 5.8 GHz ISM band. For example, the length of radiating element 262 may be three quarters of the effective wavelength at the 915 MHz ISM band. The effective wavelength is dependent on the medium surrounding the antenna and the design of a dielectric covering on the radiating element 262. The design of the dielectric covering includes features such as the type of dielectric(s) and thickness of the dielectric layer(s). The exact length of the radiating element 262 may be designed to get good impedance matching.

In any of the embodiments herein, one or more outer surfaces of radiating element 262 may be covered with one or more layers of antenna dielectrics 266. The thickness and type of antenna dielectrics 266 along the length of radiating element 262 may be designed to modify and optimize the microwave properties of the antenna 112. For example, one or more antenna dielectrics 266 covering radiating element 262 may be used to shape the microwave field and to optimize the performance of antenna 112. The one or more antenna dielectrics 266 covering radiating element 262 may be used to shape the microwave field by changing the local dielectric environment in the region adjacent to radiating element 262. Examples of dielectric materials that can be used to design one or more embodiments disclosed herein include, but are not limited to EPTFE, PTFE, FEP and other floropolymers, Silicone, Air, PEEK, polyimides, cyanoacrylates, epoxies, natural or artificial rubbers and combinations thereof. In one embodiment, entire radiating element 262 is covered with a silicone dielectric. The layer of silicone used to coat a distal portion of radiating element 262 may be thinner than the layer of silicone used to coat a proximal portion of radiating element 262. The thinner silicone dielectric may be used to compensate for the lower field strength that normally exists at the distal portion of a microwave antenna. Thus, the microwave field is made more uniform along the length of radiating element 262.

In any of the embodiments herein, the shape of radiating element 262 may be that same or different from the shape of shaping element 264. Further in any of the embodiments herein, both radiating element 262 and shaping element 264 may be non-linear. Further, in any of the embodiments herein, radiating element 262 and shaping element 264 may be parallel or non-parallel to each other.

In the embodiment shown in FIG. 1A, the novel microwave field shaping technique of the present invention is used to improve the performance of a helical antenna. The resultant antenna can be used to create a uniform lesion along the length of the antenna without adversely affecting tissues surrounding the transmission line. In the embodiment shown in FIG. 1A, the width of antenna 112 is substantially the same as the width of the coaxial cable 250. In one embodiment, a single microwave signal is fed to antenna 112 through coaxial cable 250. Antenna 112 generates a microwave field. The near field of the microwave field generated by antenna 112 may be used for achieving the desired clinical outcome such as ablating tissue. In FIG. 1A, antenna 112 comprises a radiating element 262 and a shaping element 264. In one embodiment, radiating element 262 is a continuation of the inner conductor 258 of coaxial cable 250. Shaping element 264 shapes the microwave field emitted by radiating element 262. In one embodiment, shaping element 264 is made of an electrically conductive material e.g. a metal or a conductive polymer and is electrically connected to a region of outer conductor 256 of coaxial cable 250. In an alternate embodiment, a conductive shaping element 264 is electrically isolated from outer conductor 256. In this embodiment, shaping element 264 functions as a passive radiator or parasitic element of antenna 112. Shaping element 264 in this electrically isolated embodiment absorbs microwaves radiated from radiating element 262 and re-radiates microwaves. It should be noted that there is no direct electrical conduction between radiating element 262 and shaping element 264. When microwave energy is delivered through coaxial cable 250 to antenna 112 in FIG. 1A, a first microwave field is emitted by radiating element 262. This first microwave field is a normal mode microwave field of a small diameter (antenna diameter D is much less than the microwave wavelength) helical antenna. The first microwave field interacts or couples with shaping element 264. This interaction induces a leakage current on shaping element 264. The leakage current in turn creates a second microwave field. The second microwave field is an elongated, axial mode microwave field due to the elongate shape of shaping element 264. The first microwave field and the second microwave field together combine to produce a unique shaped microwave field of antenna 112 that is clinically more useful that the unshaped microwave field generated by an antenna 112 comprising only radiating element 262. Thus the original microwave field is redistributed by the design of shaping element 264. Shaping element 264 alone is not capable of functioning as an antenna; rather shaping element 264 shapes or redistributes the electromagnetic or microwave field emitted by radiating element 262 to produce a clinically improved microwave field. It should be noted that there is no direct electrical conduction between radiating element 262 and shaping element 264 in FIG. 1A.

Further, the specific design of shaping element 264 may be used to improve the power deposition of an antenna 112 comprising radiating element 262. Shaping element 264 may be made of one or more non-insulated or insulated conducting materials e.g. metals, conductive polymers, materials with embedded conductive particles, etc. The embodiments of the present invention may be designed wherein individual elements e.g. radiating element 262 have minimal or no sharp corners to avoid undesirable regions of concentrated microwave field.

Antenna 112 in FIG. 1A has a linear, elongate shape that is especially suited for the ablation of a linear region of tissue e.g. for the creation of a linear lesion in the left atrium or a hollow organ such as a blood vessel. Alternately, a linear, flexible antenna 112 can be bent in a variety of shapes as disclosed subsequently in this specification for the creation of various non-linear lesions.

In FIG. 1A, every portion of radiating element 262 is covered with some dielectric material such that no metallic surface of radiating element 262 is exposed to tissue. Thus, in this embodiment, radiating element 262 is electrically insulated from tissue. Thus, in this embodiment, radiating element 262 is able to transmit a microwave field into tissue, but unable to conduct electricity to tissue. Thus, in this embodiment, there is no electrical conduction and no conductive path between radiating element 262 and shaping element 264. Further, in this embodiment, there is no electrical conduction and no conductive path between radiating element 262 and the surrounding tissue. In one embodiment, the dielectric on a proximal portion of radiating element 262 is a continuation of the dielectric 260 of coaxial cable 250. The thickness of a dielectric on radiating element 262 may vary along the length of radiating element 262. Further, the cross section of a dielectric on radiating element 262 may not be radially symmetric.

In the embodiment of FIG. 1A, radiating element 262 is made of a helically arranged length of a metallic conductor. The helix may be symmetric with a constant pitch and a constant diameter along the length of the helix. In one embodiment, the straightened length of the conductor used for constructing radiating element 262 is about three quarters of the effective wavelength at 915 MHz. In alternate embodiments, this length may be an odd multiple of one quarter of the effective wavelength at one of: 433 MHz ISM band, 915 MHz ISM band, 2.45 GHz ISM band and 5.8 GHz ISM band. Although in FIG. 1A, radiating element 262 has about 19 turns, embodiments of ablation devices 110 may be constructed wherein radiating element 262 has about 1 to 30 turns. The pitch of a helical radiating element 262 may range between 0.3 mm and 20 mm Radiating element 262 may be made from a metallic element or alloy selected from the group comprising Nitinol, stainless steel or copper. Radiating element 262 may comprise a plating of a conducting metal such as silver or gold on the outer surface of radiating element 262. The metallic conductor used for constructing radiating element 262 may have a round, oval, rectangular or square cross section. In one embodiment, the metallic conductor used for constructing radiating element 262 has a round cross section with a diameter of 0.5 mm+/−0.4 mm. In another embodiment, the metallic conductor used for constructing radiating element 262 has a rectangular cross section with cross sectional dimensions of 10 mm+/−9.5 mm by 0.5 mm+/−0.4 mm. In another embodiment of a radiating element with a rectangular cross section, the cross sectional dimensions are 1 mm+/−0.3 mm by 0.1 mm+/−0.05 mm. In an alternate embodiment, radiating element 262 is made of a length of a metallic conductor that is arranged in a substantially two dimensional configuration. For example, the length of a metallic conductor may be arranged in a substantially wavy or zigzag or serpentine configuration. In the embodiment in FIG. 1A, radiating element 262 is arranged symmetrically around shaping element 264 and partially or fully encloses shaping element 264. Shaping element 264 may be made of a linear or helical length of a metallic conductor. The outer diameter of shaping element 264 may be uniform or may be non-uniform along the length of antenna 112. In the embodiment shown in FIG. 1A, shaping element 264 is made of a substantially linear length of a metallic conductor. Shaping element 264 may be made from a metallic element or alloy selected from the group comprising Nitinol, stainless steel or copper. Shaping element 264 may comprise a plating of a conducting metal such as silver or gold on the outer surface of shaping element 264. The metallic conductor used for constructing shaping element 264 may have a round, oval, rectangular or square cross section. In one embodiment, the metallic conductor used for constructing shaping element 264 has a round cross section with a diameter of 0.5 mm+/−0.3 mm. In another embodiment, the metallic conductor used for constructing shaping element 264 has a rectangular crossection with dimensions of 0.5 mm+/−0.3 mm by 0.5 mm+/−0.3 mm. Antenna 112 further comprises one or more antenna dielectrics 266 between radiating element 262 and shaping element 264. In one embodiment, antenna dielectric 266 is sufficiently flexible to create a flexible antenna 112. The flexibility of antenna 112 allows antenna 112 to bend from a substantially linear configuration to a substantially non-linear configuration and vice-versa during clinical use. The flexibility of antenna 112 also allows antenna 112 to bend relative to the distal end of the transmission line during clinical use. This in turn allows a user to introduce antenna 112 to the target location through tortuous or non-linear introduction paths such as blood vessels and also to create non-linear lesions. In one embodiment, antenna dielectric 266 is sufficiently stiff to create a sufficiently stiff antenna 112. The stiffness of antenna 112 prevents antenna 112 from bending during clinical use. This in turn enables the user to use antenna 112 to puncture or penetrate through tissue such as tumor tissue. Examples of dielectrics that can be used between radiating element 262 and shaping element 264 include, but are not limited to EPTFE, PTFE, FEP and other floropolymers, Silicone, Air, PEEK, polyimides, natural or artificial rubbers and combinations thereof. Additionally the entire antenna 112 may be covered or encapsulated in a dielectric. Examples of dielectrics that can be used to cover or encapsulate antenna 112 include, but are not limited to EPTFE, PTFE, FEP and other floropolymers, Silicone, PEEK, polyimides, natural or artificial rubbers and combinations thereof. Antenna dielectric 266 may comprise one or more layers of such dielectrics. The dielectric used to cover or encapsulate antenna 112 may be porous or non-porous. In the embodiment shown in FIG. 1A, the length of antenna 112 is between 10 mm and 80 mm. In FIG. 1A, the width of antenna 112 is between 1 mm and 40 mm. In one particular embodiment, antenna 112 has a length of 45 mm+/−7 mm and a width of 2 mm+/−0.5 mm. Radiating element 262 is electrically connected to inner conductor 258 of coaxial cable 250. This may be done for example, by soldering or resistance welding radiating element 262 to inner conductor 258. Shaping element 264 is electrically connected to outer conductor 256 of coaxial cable 250. This may be done for example, by soldering or resistance welding shaping element 264 to outer conductor 256. Antenna 112 may be floppy, flexible or substantially rigid. Antenna 112 may be malleable or have shape memory or elastic or super-elastic properties. The distal end of antenna 112 may be atraumatic. Antenna 112 may be designed such that the length of antenna 112 is adjustable. For example, length of antenna 112 may be increased or reduced to increase or reduce the length of an ablation zone. In this embodiment, shaping element 264 may have a helical or substantially wavy or zigzag or serpentine configuration. The length of antenna 112 may be increased or reduced intra-operatively or pre-operatively. In one embodiment, one or both of radiating element 262 and shaping element 264 are a part of a flexible circuit and are manufactured using commonly known techniques for manufacturing flexible circuits.

In FIG. 1A, the shape of radiating element 262 is different from the shape of shaping element 264. Further in the embodiment in FIG. 1A, radiating element 262 is non-linear. Further in the embodiment in FIG. 1A, shaping element 264 is substantially linear. However radiating element 262 and shaping element 264 are generally oriented such that their axes are parallel to each other. Alternate embodiments of antenna 112 may be designed wherein radiating element 262 is substantially linear. Alternate embodiments of antenna 112 may be designed wherein shaping element 264 is substantially non-linear. Alternate embodiments of antenna 112 may be designed wherein radiating element 262 and shaping element 264 are generally oriented such that their axes are not parallel.

Although in the embodiment in FIG. 1A shaping element 264 is connected to the distal end of coaxial cable 250, other embodiments of antenna 112 may be designed wherein shaping element 264 is connected to coaxial cable 250 at a region other than the distal end of coaxial cable 250. For example, in one alternate embodiment, shaping element 264 is metallic and is electrically connected to a region of outer conductor 256 of coaxial cable 250 proximal to the distal end of the coaxial cable 250.

In FIG. 1A, since radiating element 262 is in electrical contact with inner conductor 258, there is a first electrically conductive path extending from inner conductor 258 till the distal end of radiating element 262. In the embodiments wherein shaping element 264 is made of a conductive material and is electrically connected to outer conductor 256 of coaxial cable 250, there is a second electrically conductive path extending from outer conductor 256 till the distal end of shaping element 264. In such embodiments, even though there are two conductive paths that extend from first zone Z1 to the second zone Z2, the designs, materials and the microwave properties of the two conductive paths may be significantly different in first zone Z1 and second zone Z2 as described before. In first zone Z1, outer conductor 256 of coaxial cable 250 is located symmetrically around inner conductor 258 and at a constant distance from inner conductor 258. However, in second zone Z2, radiating element 262 is located symmetrically around shaping element 264 and at a constant distance from shaping element 264. In first zone Z1, outer conductor 256 of coaxial cable 250 always acts as a shield for the microwave field in first zone Z1 whereas in second zone Z2, shaping element 264 may or may not act as a shield for the microwave field in second zone Z2.

FIG. 1C shows a section through an embodiment of coaxial cable 250 usable for ablation device 110 of FIG. 1A, 1B and for other ablation devices 110 disclosed herein. In one embodiment, coaxial cable 250 used herein is flexible and comprises an inner conductor 258 made of Nitinol with a Ni content of 56%+/−5%. The outer diameter of inner conductor 258 is 0.0172″+/−0.004″ Inner conductor 258 has a cladding or plating 270 of a highly conductive metal such as silver or gold. In one embodiment, inner conductor 258 comprises a silver cladding 270 of thickness 0.000250″+/−0.000050″. Cladding 270 in turn is surrounded by dielectric material 260. In one embodiment, dielectric material 260 is made of expanded PTFE with an outer diameter of 0.046″+/−0.005″. The dielectric material 260 in turn is surrounded by the outer conductor 256. Outer conductor 256 acts as a shielding element to the microwave signals transmitted by inner conductor 258. Further, outer conductor 256 shields the microwave signals transmitted by inner conductor 258 from external noise. In one embodiment, outer conductor 256 comprises multiple strands of silver plated copper. The multiple strands of outer conductor 256 are arranged such that the outer diameter of outer conductor 256 is 0.057″+/−0.005″. Outer conductor 256 in turn is covered by an outer jacket 268. In one embodiment, outer jacket 268 is made of PTFE with an outer diameter of 0.065″+/−0.005″. Thus, the outer diameter of coaxial cable 250 is less than about 2 mm. The low profile of flexible coaxial cable 250 has tremendous clinical advantages since it can be inserted through narrow and/or tortuous anatomical paths or introducing device lumens. In one embodiment, a shaft comprising coaxial cable 250 is stiffened or strengthened by adding one or more stiffening or strengthening elements such as enclosing stiffening devices jackets, braids, or stiffening layers over coaxial cable 250. In one embodiment, antenna 112 is stiffened or strengthened by adding one or more stiffening or strengthening elements such as jackets, braids or layers within or over antenna 112.

FIG. 1D shows a longitudinal section of the embodiment of ablation device 110 of FIG. 1A through the distal end of coaxial cable 250. In FIG. 1D, the identity of coaxial cable 250 ends at the distal end of outer conductor 256. Transition line 254 in FIG. 1D is located at the distal end of outer conductor 256 and is perpendicular to the axis of coaxial cable 250 at the distal end of outer conductor 256. Outer jacket 268 of coaxial cable 250 terminates a small distance proximal to the distal end of outer conductor 256. A conductive element attached to the distal end of inner conductor 258 forms radiating element 262. In one embodiment, the proximal end of radiating element 262 is electrically connected to the distal end of inner conductor 258. In one embodiment, the proximal end of radiating element 262 is soldered to inner conductor 258. In another embodiment, the proximal end of radiating element 262 is laser welded to inner conductor 258. The proximal end of radiating element 262 may be electrically connected to inner conductor 258 in various configurations including, but not limited to lap joint and butt joint. The proximal end of shaping element 264 is electrically connected to a region of outer conductor 256. In one embodiment, the proximal end of shaping element 264 is electrically connected to the distal end of outer conductor 256. In one embodiment, the proximal end of shaping element 264 is soldered to outer conductor 256. In another embodiment, the proximal end of shaping element 264 is laser welded to outer conductor 256. The proximal end of shaping element 264 may be electrically connected to outer conductor 256 in various configurations including, but not limited to lap joint and butt joint.

FIG. 1B shows a side view of an embodiment of a microwave antenna comprising one or more functional elements such as electrophysiological mapping or pacing or RF ablation electrodes. Antenna 112 in FIG. 1B is similar in design to antenna 112 of FIG. 1A. Antenna 112 in FIG. 1B has a proximal end connected to the distal end of the transmission line 250, a distal end and an antenna axis. However, antenna 112 in FIG. 1B further comprises one or more functional elements 118. Several embodiments of the present invention will be described herein using electrodes as examples of functional elements 118. Electrode 118 may be used for one or more of: sensing and/or measuring electrophysiological signals, electrically pacing a tissue and ablating an anatomical region. In the embodiment wherein antenna 112 is used to treat or ablate tissue, electrode 118 may be used for one or more of: identifying the site of treatment, measuring tissue impedance, confirming successful treatment, and monitoring progress of the treatment. In one method embodiment, ablation device 110 is introduced percutaneously into the heart and is used to ablate cardiac tissue to treat an arrhythmia such as atrial fibrillation, flutter, ventricular tachycardia, etc. In another method embodiment, ablation device 110 is introduced percutaneously into the renal arteries and is used to ablate the vasculature to treat hypertension and other disorders. In such methods, electrodes 118 are used for one or more of: identifying the site of the arrhythmia, identifying site(s) to be ablated, measuring tissue impedance, confirming successful ablation of the cardiac tissue, confirming uni-directional or bi-directional block and monitoring progress of the ablation. Thus electrodes 118 confer a clinical advantage to the physician since ablation and sensing can be done by a single slim and flexible device. Electrodes 118 are made of a metallic material and are exposed to the surrounding tissue. In FIG. 1B, electrode 118 is located in the microwave field generated by antenna 112. Electrode 118 is connected by an elongate conductive wire 276 that electrically connects electrode 118 to an external circuit. Wire 276 is located in the microwave field generated by antenna 112. If improperly placed, both electrode 118 and wire 276 can cause unwanted coupling with the microwave field which in turn can cause unwanted distortion of the microwave field. FIG. 1B shows one embodiment of conductive wire 276 that are designed to cause minimal or no interference with the microwave energy emitted by antenna 112. In FIG. 1B, electrode wire 276 is arranged in a helical configuration around shaping element 264. This novel arrangement or conductive wire 276 reduces the coupling between the conductive wire 276 and the microwave field which in turn reduces the heating of conductive wire 276. Conductive wire 276 may be made of one or more conductive materials. Examples of such materials include, but are not limited to: stainless steel (with or without a coating of copper or gold or silver), copper, gold, silver and alloys thereof and other conductive materials. The diameter of the conductive wire 276 helix in FIG. 1B is substantially uniform along the length of antenna 112. Further, the pitch (distance between adjacent turns) is substantially similar to the pitch of radiating element 262. Further, the axis of the conductive wire 276 helix in FIG. 1B is oriented substantially parallel to the axis of antenna 112. In one embodiment, the diameter of the helical arrangement of conductive wire 276 is less than the diameter of radiating element 262. In an alternate embodiment, conductive wire 276 is substantially straight and is arranged non-parallel to the length of antenna 112. In one embodiment, the axis of helically disposed conductive wire 276 is substantially parallel to the axis of one or both of: radiating element 262 and shaping elements 264. In FIG. 1B, if conductive wire 276 is defined as a series of curved segments, all of the curved segments are oriented such that they are non-parallel to the axis of antenna 112. The portion of wire 276 located on antenna 112 has about 9 turns. In alternate embodiments, the portion of wire 276 located on antenna 112 has about 0.5 to 30 turns. In another embodiment, coupling between the microwave field and conductive wire 276 is reduced by spacing conductive wire 276 from radiating element 262 by a sufficient amount of a dielectric material. In one embodiment, coupling between the microwave field and conductive wire 276 is used to purposely modify the shape and/or the size of the microwave field. In one embodiment, one or more conductive wires 276 pass along the proximal end of antenna 112. In one such embodiment, one or more conductive wires 276 are routed over and along coaxial cable 250 i.e. proximally from antenna 112. In another embodiment, one or more conductive wires 276 pass along the distal end of antenna 112. In one such embodiment, one or more conductive wires 276 are routed distally from antenna 112 through a distal dielectric 278 as shown in FIG. 1B. Distal dielectric 278 is located on the distal end of ablation device 110. In one embodiment, one or more conductive wires 276 are embedded in distal dielectric 278. Distal dielectric 278 may or may not form a part of a pull wire or tether 114 arrangement located on the distal region of ablation device 110 as shown in FIGS. 1AA and 1AB. In one embodiment, conductive wires 276 themselves are designed to be tethers 114 or pull wires. In another embodiment, a separate flexible tether 114 is attached to the distal region of ablation device 110 and conductive wires 276 are arranged along tether 114 such that there is no or minimal tension on conductive wires 276 during clinical use.

Electrode 118 is designed to be as small as possible. In one embodiment, electrode 118 is made of a metallic cylindrical ring with an outer diameter between 0.5-5 mm and a length between 0.1-4 mm. In one embodiment, coupling between the microwave field and electrode 118 is reduced by placing electrode 118 in a region of antenna 112 where the microwave field is relatively weak. In another embodiment, coupling between the microwave field and electrode 118 is reduced by spacing electrode 118 from radiating element 262 by a sufficient amount of a dielectric material. Although only a single electrode 118 is shown in FIG. 1B for clarity, embodiments of ablation device 110 may be designed that comprise more than one electrode 118. In one embodiment, an antenna 112 comprising multiple electrodes 118 is designed such that even though individual electrodes 118 may interact with and slightly distort the microwave field, the combined effect of multiple electrodes 118 causes minimal or no net microwave field distortion.

FIGS. 1E-1G show the method steps of a method of creating two overlapping lesions in a tissue. In FIGS. 1E-1G, the method is demonstrated in an experimental setup for demonstrating the utility of antenna 112 of FIG. 1A for intra-cardiac ablation, renal denervation, and other applications. The experimental setup comprised a slice of porcine muscle tissue is kept in a water bath maintained at 37 C. Further, water is pumped in the water bath from a nozzle 272 and is continuously circulated through the water bath using a pump (not shown). This is to simulate the effect of blood flow. In FIG. 1E, a linear antenna 112 similar in design to antenna 112 of FIG. 1A is placed in contact with the porcine tissue as shown. Thereafter, microwave power at 0.915 GHz is delivered to ablation device 110 at 80 W for 60 s. FIG. 1E shows a first ablation created around antenna 112. In FIG. 1F, antenna 112 is moved to a new location. Thereafter, microwave power is delivered to ablation device 110 at 80 W for 60 s to create a second lesion as shown in FIG. 1G. In FIG. 1G, antenna 112 is being moved away after creating the second lesion. The tissue shows two overlapping lesions that overlap lengthwise. Various patterns of multiple lesions may thus be created by repositioning any of the antennas 112 disclosed herein. Any of the antennas 112 disclosed herein may be repositioned by one or more of: rotating around an axis, moving proximally or distally, moving sideways, revolving around an axis, increasing or reducing in size (for example based on an anatomical dimension), engaging a steering or deflecting mechanism on ablation device 110 and engaging a steering or deflecting mechanism on an accessory device. Further, any of the antennas 112 disclosed herein may be designed and used such that during clinical use the forces exerted by a flexible antenna 112 on surrounding tissues do not distort the surrounding tissue. In one embodiment, two lesions are created that do not intersect each other. In another embodiment, two elongate lesions are created that are joined lengthwise. In another embodiment, two elongate lesions are created that are joined breadthwise. In another embodiment, two elongate lesions are created that intersect each other to form an approximately X-shaped resulting lesion.

The lesions created in FIG. 1G were deep and overlapping lesions lacking any “gaps” within a lesion of unablated tissue. The length of the combined lesion was about 9 cm and the visual depth of the lesion varied from 1-1.5 cm. Thus, long, deep lesions may be created by antenna 112. The lesions may be created such that they span the entire thickness of the tissue such as a heart wall. Thus antenna 112 can be used to create trans-mural lesions. Further, there is a complete absence of charring in the lesion. Also, long, deep lesions were created even in the presence of flowing fluid. Thus antenna 112 may be used to create lesions in anatomical regions that contain flowing blood such as the vasculature (veins, arteries, etc.) and the heart chambers. Using other treatment settings, similar elongate lesions can be created that range in depth from 3-10 mm.

FIG. 1H shows a side view of an embodiment of a microwave antenna comprising multiple electrophysiological mapping or pacing electrodes that are spaced in regular intervals along the length of antenna 112. Embodiments of ablation device 110 may be designed that comprise 1-64 such electrodes 118. Electrodes 118 may be spaced at regular intervals or at non-regular intervals along the length of antenna 112. In any of the embodiments of electrodes 118 disclosed herein wherein electrodes 118 are electrophysiological mapping electrodes; one or more electrodes 118 may be used in a uni-polar mode or a bi-polar mode. When such electrodes 118 are used in a bi-polar mode, any two electrodes 118 (e.g. two adjacent electrodes 118) may be used for performing a mapping procedure. For example, in FIG. 1H, electrodes 118 may be used in the bi-polar mode by using first and second electrodes 118, second and third electrodes 118, third and fourth electrodes 118, etc. In one embodiment, an antenna 112 comprising multiple electrodes 118 such as shown in FIG. 1H is designed such that even though individual electrodes 118 may interact with and slightly distort the microwave field, the combined effect of multiple electrodes 118 causes minimal or no net microwave field distortion.

FIG. 1I shows a side view of an embodiment of a microwave antenna comprising multiple electrophysiological mapping or pacing electrodes that are spaced in pairs along the length of antenna 112. Such an embodiment is especially suited for cardiac ablation using antenna 112 wherein electrodes 118 are used for electrophysiological mapping in a bi-polar mode. In such an embodiment, two electrodes 118 in a pair are used in a bi-polar mode for performing an electrophysiological mapping procedure. In FIGS. 1H and 1I, conductive wires 276 are not shown for improved clarity.

Although in several figures, elements 118 are described as electrophysiological mapping electrodes 118, one or more of electrodes 118 in any of the embodiments shown herein (such as those shown in FIGS. 1B, 1H, 1I, 1K, 1S, 1T, etc.) may be one or more diagnostic or therapeutic modalities including, but not limited to: electrodes including, but not limited to: radiofrequency ablation electrodes, electrophysiological pacing electrodes, electrophysiological mapping electrodes; sensors including, but not limited to: temperature sensors, impedance sensors, pressure sensors, proximity sensors, flow sensors, moisture sensors, electromagnetic field sensors; energy emitting elements; ablation modalities designed for microwave ablation, cryoablation, laser ablation, HIFU ablation; surgical navigation elements; diagnostic elements for sensing the condition of surrounding tissue; etc. Such diagnostic or therapeutic modalities may comprise one or more metallic or conductive portions. In any of the devices disclosed herein comprising multiple such diagnostic or therapeutic modalities, all of the modalities may perform the same function. In any of the devices disclosed herein comprising multiple such diagnostic or therapeutic modalities, at least two modalities may perform different functions. For example, one modality may be an electrophysiological mapping electrode while another modality may be a temperature sensor. Such elements and the conductive wires 276 connected to such elements may be designed to have minimal or no coupling with the microwave field. Such elements and conductive wires 276 connected to such elements may be exposed to surrounding tissue or may be electrically insulated from surrounding tissue.

FIG. 1J shows a side view of a functional microwave antenna with a design similar to the design shown in FIG. 1A. Antenna 112 in FIG. 1J does not have any electrodes 118. FIG. 1K shows a side view of a functional microwave antenna having additional electrodes 118. The basic design of antenna 112 in FIG. 1K is similar to the basic design of the antenna 112 of FIG. 1J. It can be seen that the lengths of antenna 112 in FIGS. 1J and 1K are similar (about 3.5 cm). Antenna 112 in FIG. 1K comprises three pairs of electrodes 118 (a proximal pair, a middle pair and a distal pair) arranged similar to the arrangement shown in FIG. 1I. The conductive wires 276 of electrodes 118 are routed through a tether 114 connected to the distal end of antenna 112. The conductive wires 276 in FIG. 1K are arranged in a helical configuration on antenna 112 as shown in FIG. 1B. FIGS. 1L and 1M show surface images of lesions obtained by ablating the surface of porcine muscle tissue using the antennas shown in FIGS. 1J and 1K respectively. The initial temperature of the porcine muscle tissue was 37 C to simulate human body temperature. The ablation power used was 80 W and the ablation time was 80 s in both the studies in FIGS. 1L and 1M. FIGS. 1L and 1M show that the surface characteristics of the lesions are substantially similar. The length of both the lesions is about 3.8 cm. The width of both the lesions is about 7-10 mm. FIGS. 1N and 1O show sections of the porcine tissue in FIGS. 1L and 1M showing the depth of the lesions obtained by the antennas in FIGS. 1J and 1K respectively. The maximum depth of both the lesions is about 7-8 mm. This further proves that the novel positioning of electrodes 118 and/or the novel positioning of conductive wires 276 causes minimal or no interference with the microwave field emitted by antenna 112. Further, the unique arrangement of electrodes 118 and wires 276 causes a clinically insignificant distortion of the thermal profile of the heat generated by antenna 112. Further, there are no regions of charring or burning of tissue or “gaps” of unablated tissue along the lesion created in FIGS. 1M and 1O. Thus, the novel placement of electrodes 118 and conductive wires 276 has not resulted in any “hot spots” or undesired zones of high temperature.

Non-interfering designs of electrodes 118 and conductive wires 276 will be further illustrated in FIGS. 1P-1R. FIG. 1P shows a side view of a simulated SAR profile generated by an antenna similar to the antenna of FIG. 1A. FIG. 1Q shows a side view of a simulated SAR profile generated by a first antenna similar to the antenna of FIG. 1A but having four extra electrodes 118 spaced at regular intervals along the length of the antenna. The conductive wires 276 in this embodiment are arranged in a non-interfering arrangement as shown in FIG. 1B. FIG. 1Q demonstrates that the SAR profile generated by the device embodiment having four electrodes 118 is similar to the SAR profile in FIG. 1P. Thus, the unique arrangement of electrodes 118 and wires 276 causes a clinically insignificant distortion of the microwave field generated by antenna 112. Further, the unique arrangement of electrodes 118 and wires 276 causes a clinically insignificant distortion of the thermal profile of the heat generated by antenna 112. In this simulation, four conductive wires 276 were combined into a bundle of wires. Thus, the unique arrangement of conductive wires 276 causes an insignificant distortion of the microwave field generated by antenna 112. The SAR profiles in FIGS. 1Q and 1P are substantially radially symmetric. Further, the SAR profiles in FIGS. 1Q and 1P are substantially bilaterally symmetric on both sides of an imaginary plane oriented perpendicularly to the axis of antenna 112 and passing roughly through the center of antenna 112. FIG. 1Q demonstrates that the microwave field generated by antenna 112 is substantially restricted to second zone Z2. There is a clinically insignificant amount of the microwave field in first zone Z1 containing coaxial cable 250. Thus, there is negligible backward coupling between the microwave field and the distal portion of coaxial cable 250. This in turn reduces the risk of ablating tissue proximal to the distal end of coaxial cable 250. Further, the microwave field is substantially uniform along the length of antenna 112 as compared to a comparable monopole antenna. Thus the lesion formed by the microwave field in FIG. 1Q will be uniform and substantially localized. Thus, embodiments of linear antenna 112 comprising one or more electrodes 118 and their corresponding conductive wires 276 designed to operate at 915 MHz and other microwave frequencies may be designed that can create uniform, symmetrical, continuous, linear lesions with a lesion length greater than 35 mm. Further, FIG. 1Q shows the absence of undesired concentration of microwave energy over the electrodes 118 or over conductive wires 276. This in turn ensures that there will be no “hot spots” or undesired zones of high temperature along antenna 112 during clinical use. FIG. 1R shows a side view of a simulated SAR profile generated by a second antenna similar to the antenna of FIG. 1A having four extra electrodes 118 spaced at regular intervals along the length of the antenna. The conductive wires 276 in this embodiment are linear and arranged parallel to the axis of antenna 112. FIG. 1R demonstrates that the SAR profile generated by the device embodiment having four electrodes 118 is significantly different from the SAR profile in FIG. 1P. Thus, this arrangement of electrodes 118 and wires 276 has caused a clinically significant distortion of the microwave field and the thermal profile of the heat generated by antenna 112. The microwave field is concentrated in a smaller zone located near the proximal end of antenna 112. In this simulation, four conductive wires 276 were combined into a bundle of wires. The SAR profile in FIG. 1R is not bilaterally symmetric on both sides of an imaginary plane oriented perpendicularly to antenna 112 and passing roughly through the center of antenna 112. Further, the microwave field is substantially non-uniform along the length of antenna 112. Thus the lesion formed by the microwave field in FIG. 1R will be smaller than the lesion in FIG. 1Q. A comparison of the SAR profiles in FIGS. 1Q and 1R illustrate the usefulness of this unique arrangement of electrodes 118 and wires 276.

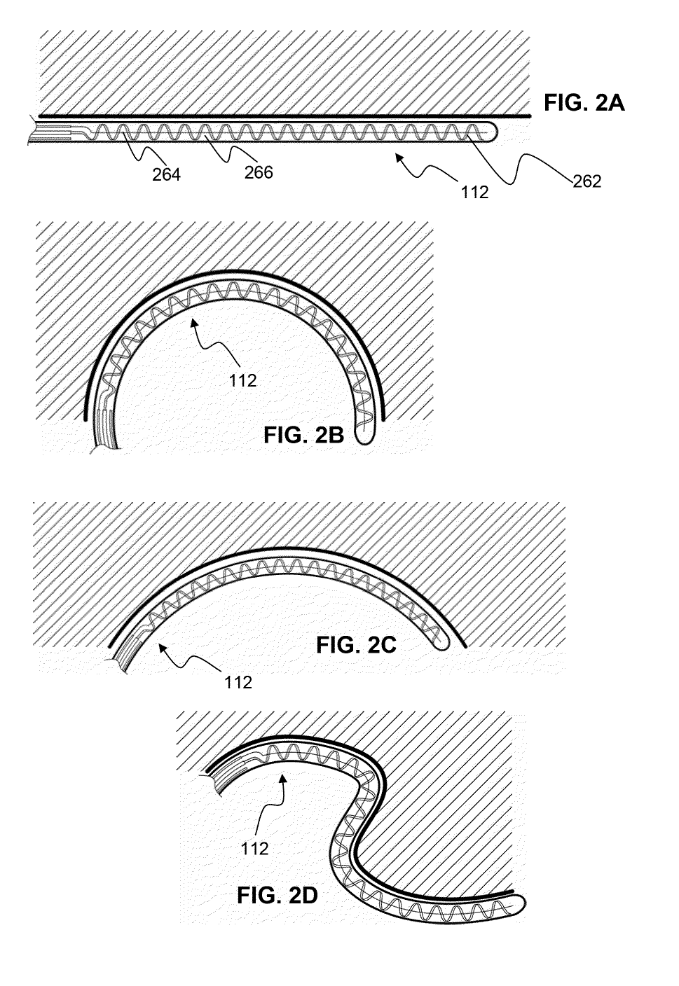

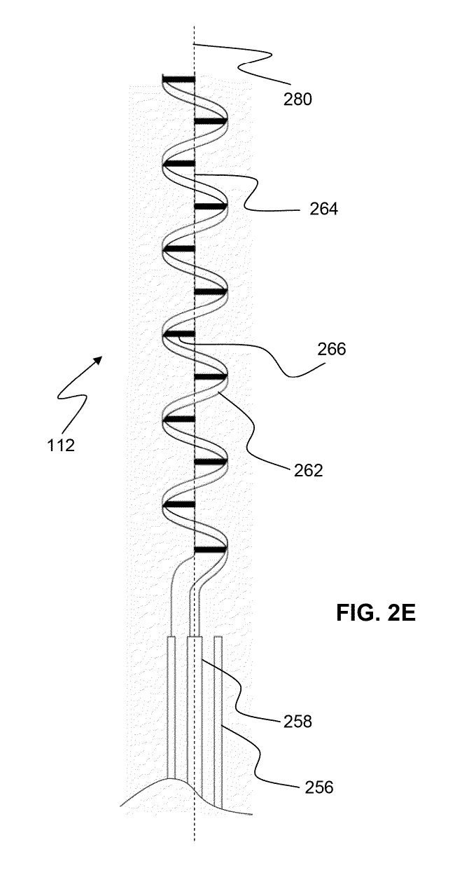

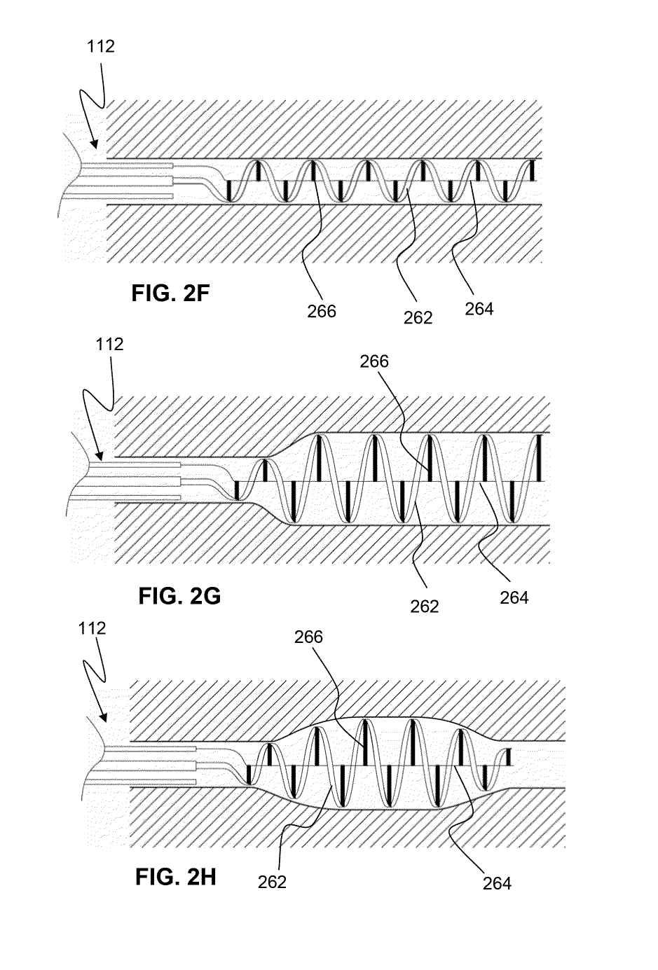

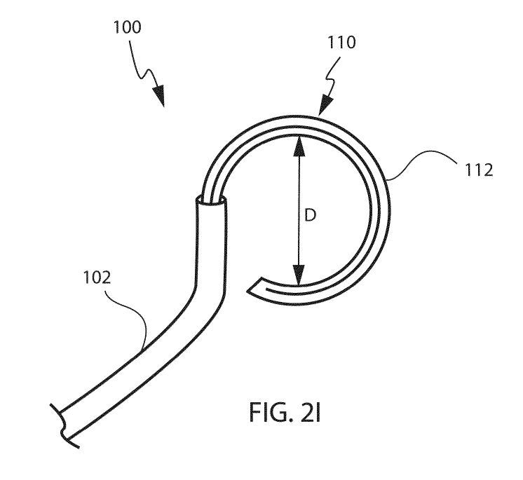

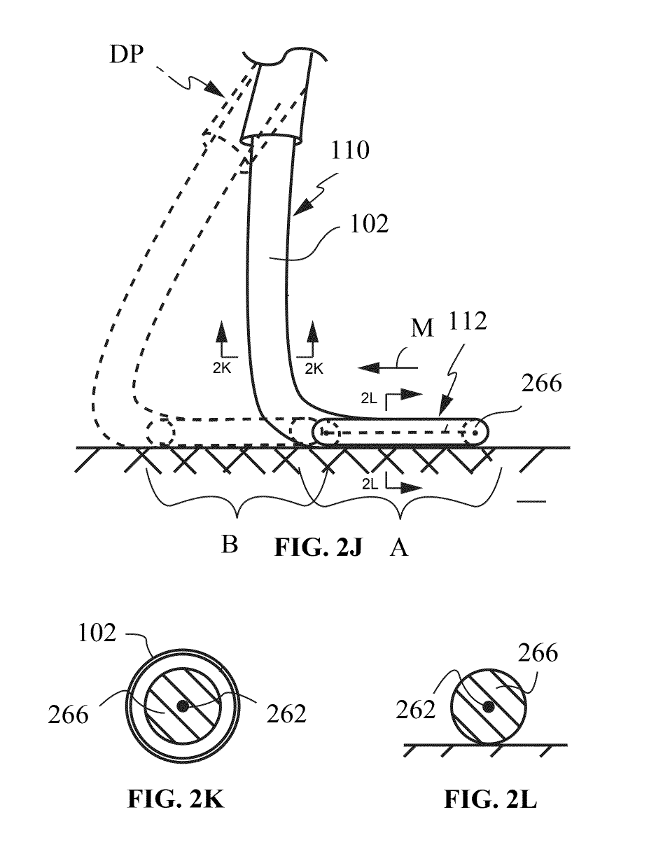

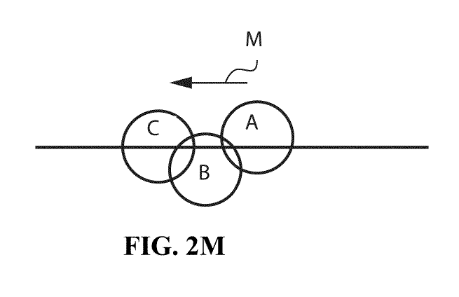

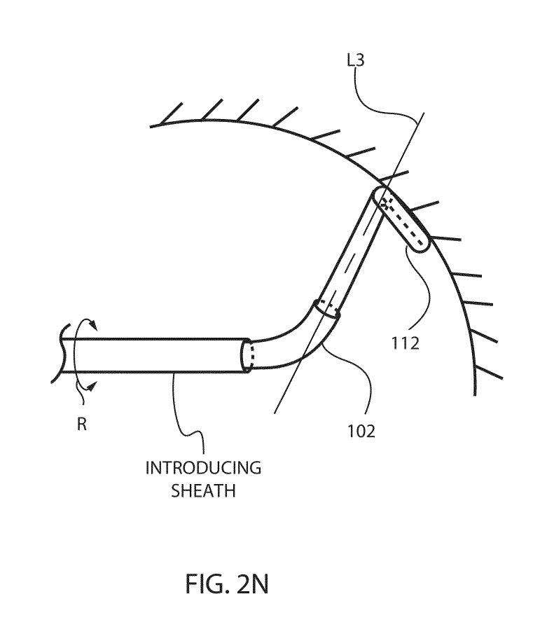

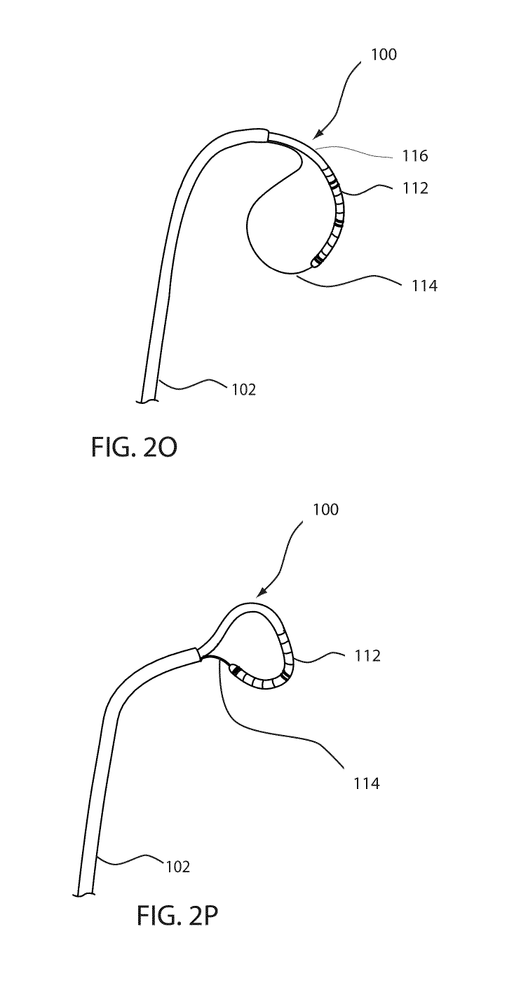

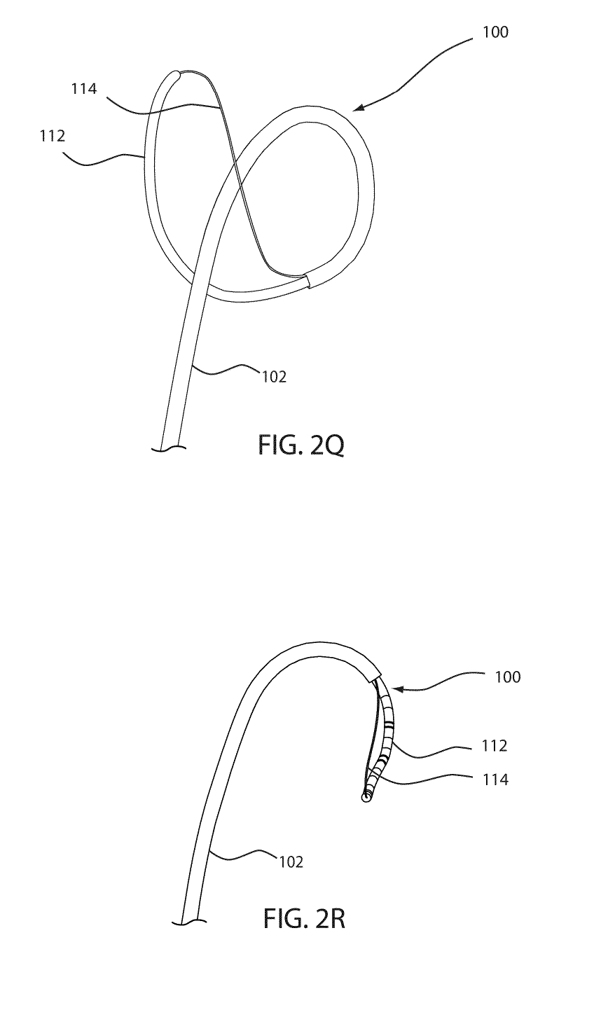

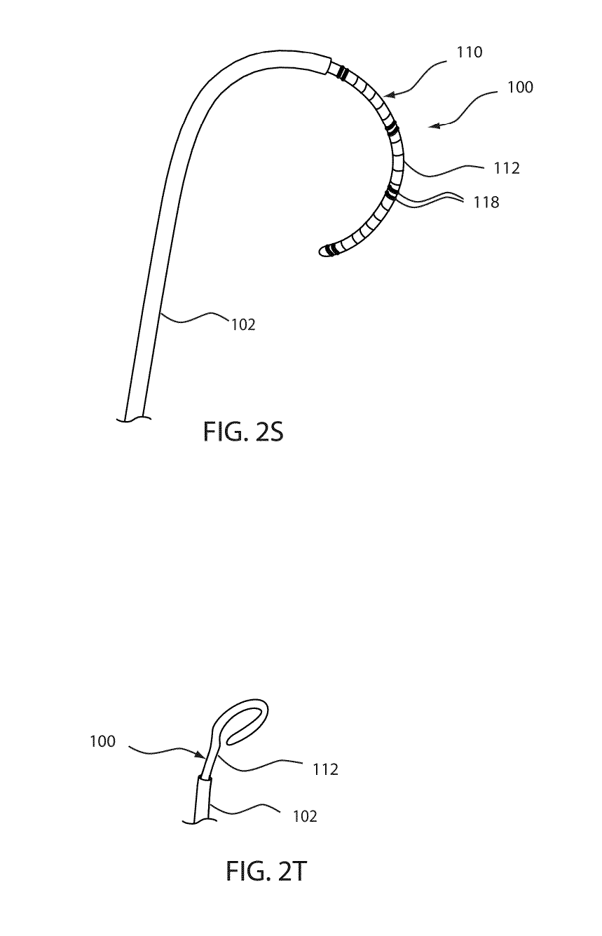

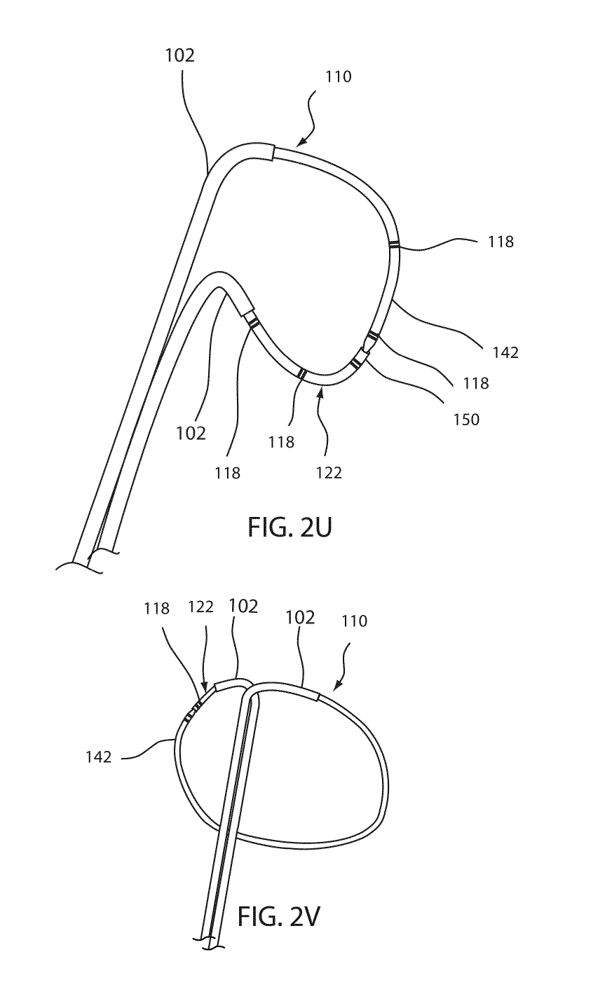

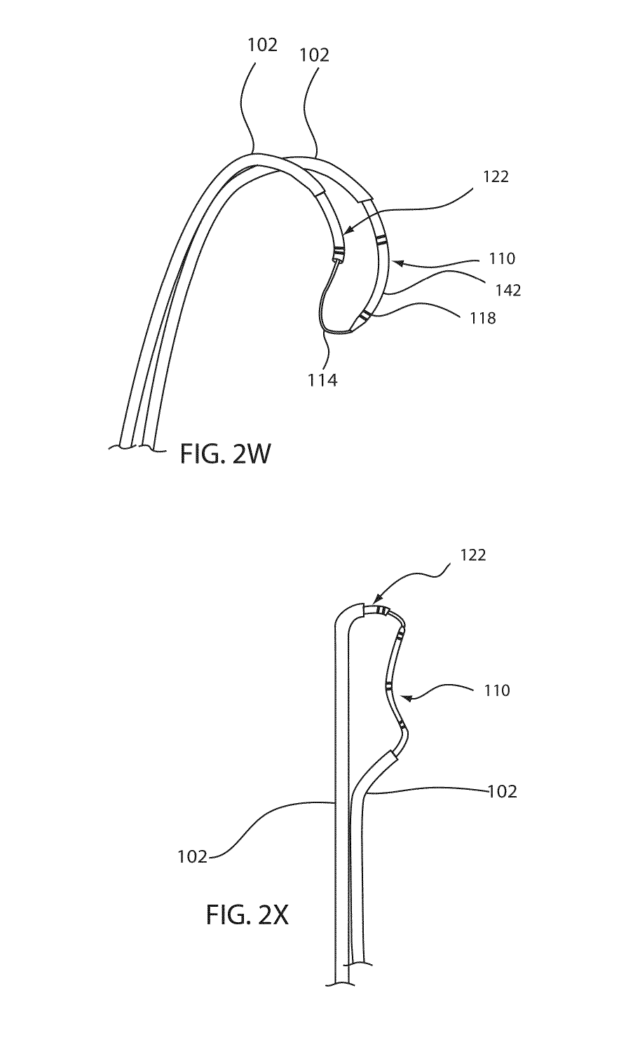

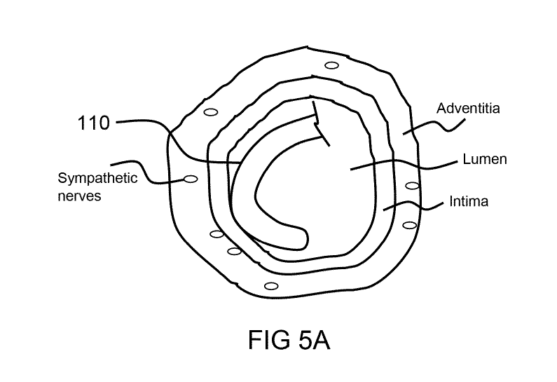

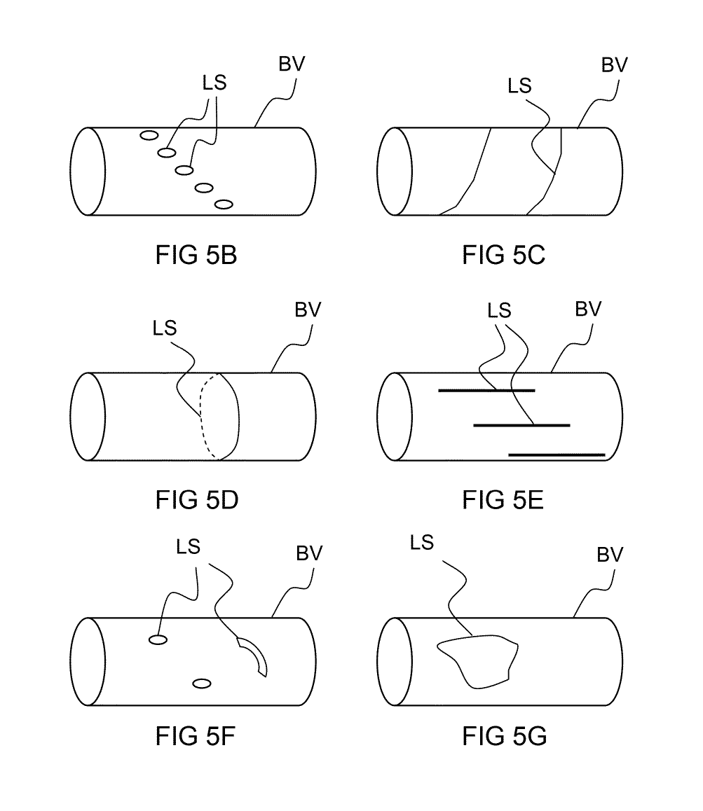

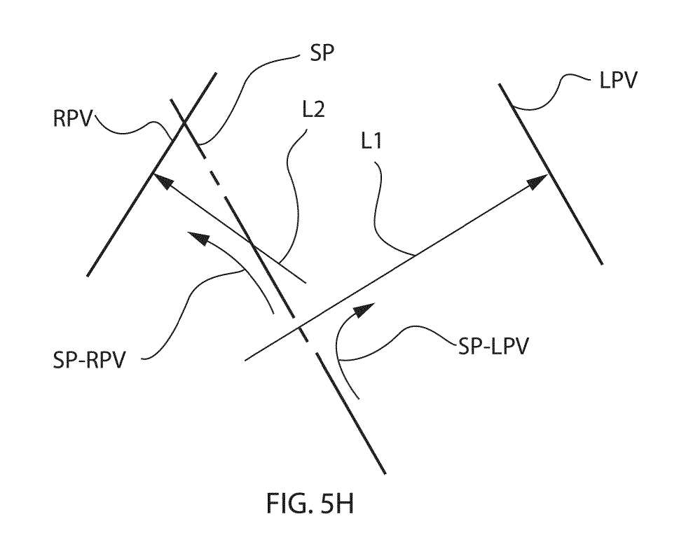

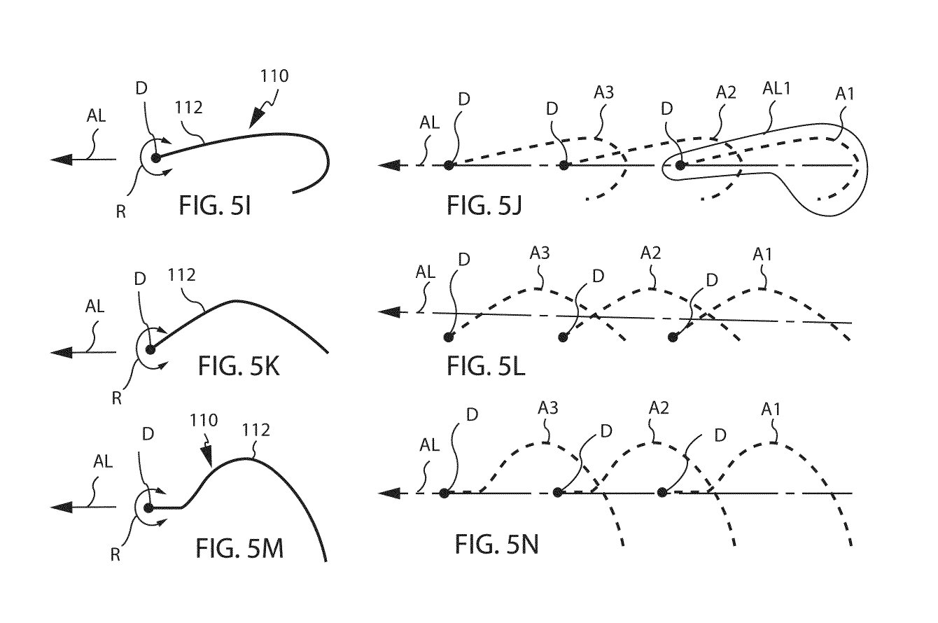

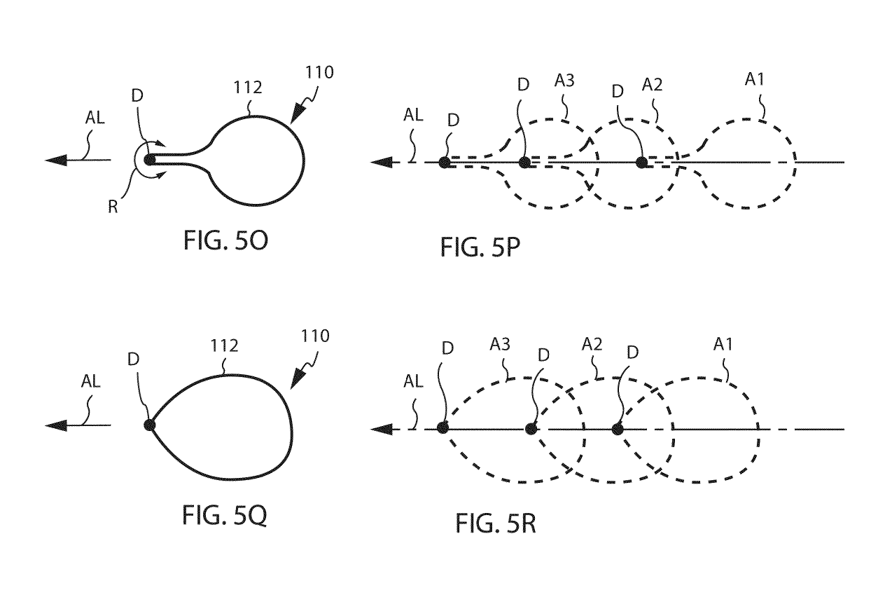

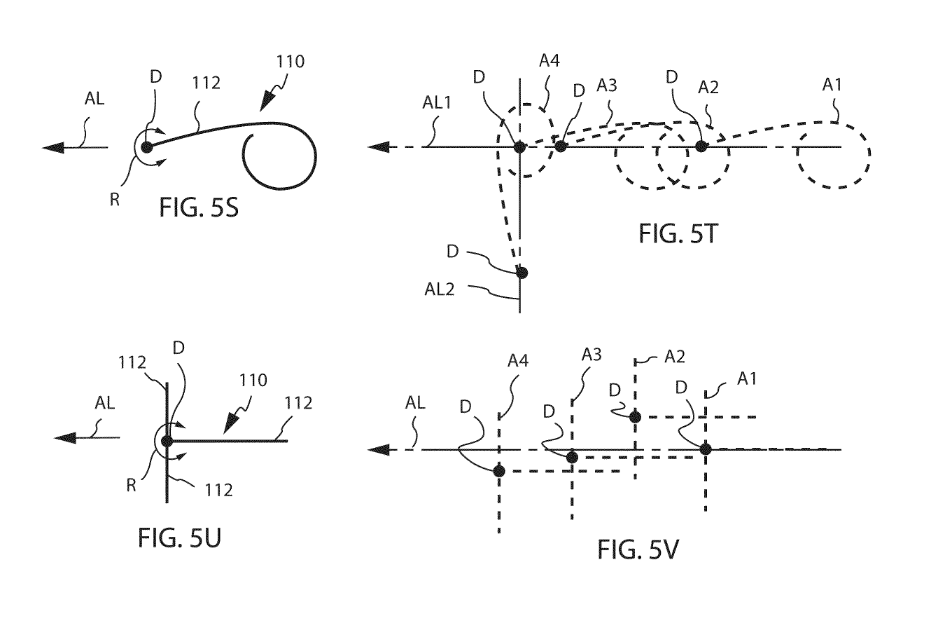

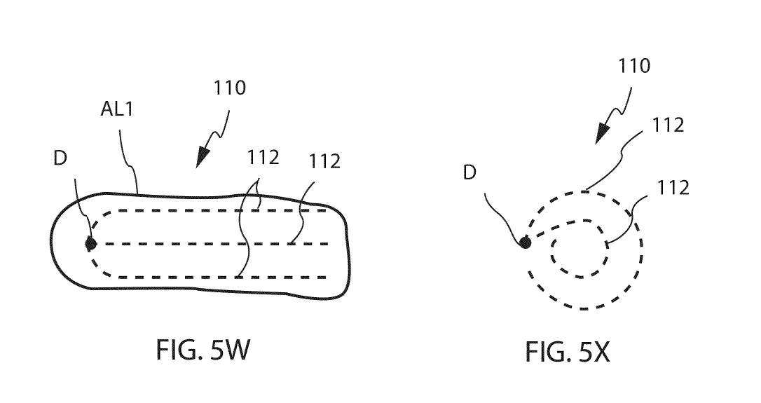

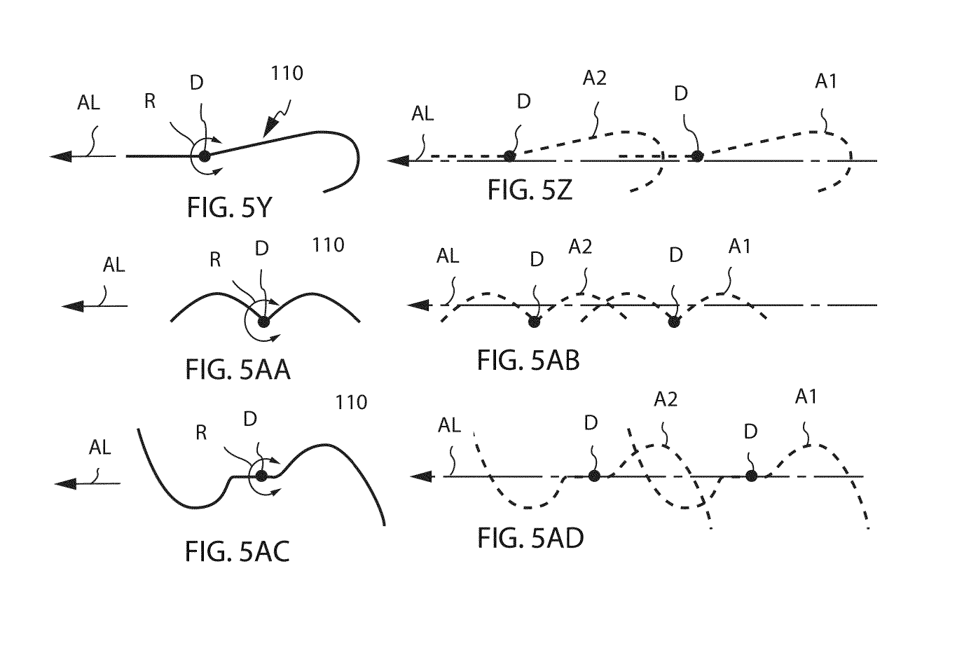

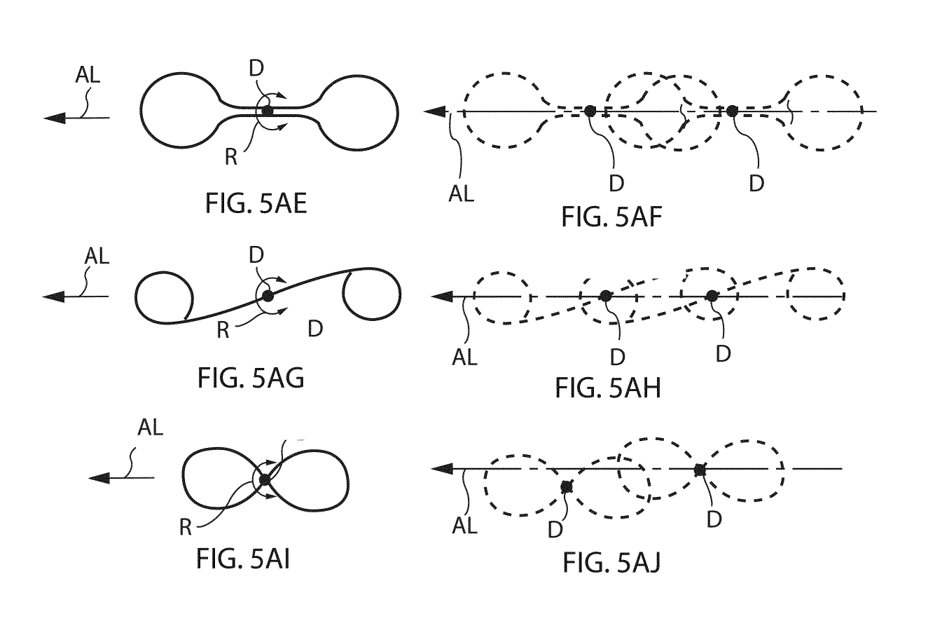

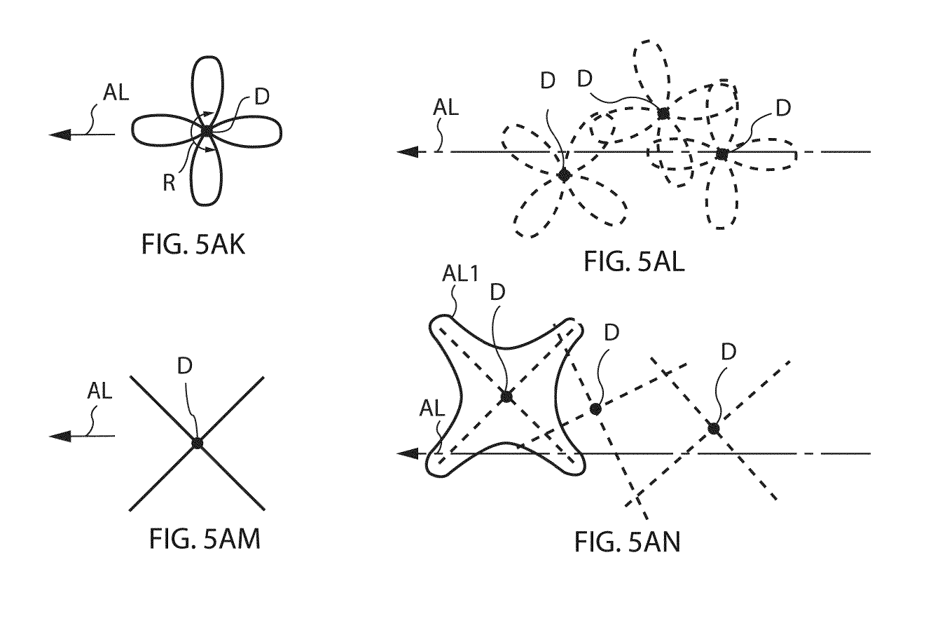

FIG. 1S shows a view of the distal region of the ablation device similar to the ablation device used in FIGS. 1K, 1M and 1O. In FIG. 1S, ablation device 110 comprises an antenna 112 similar in design to antenna 112 of FIG. 1A. Antenna 112 further comprises three pairs of electrodes 118—a proximal pair, a middle pair and a distal pair. In one embodiment, the proximal pair of electrodes 118 is located away from the junction region of antenna 112 and coaxial cable 250. In one embodiment, the distal pair of electrodes 118 is located near or on the distal end of antenna 112. A flexible coaxial cable 250 supplies power to antenna 112. The distal end of antenna 112 is connected to a tether 114 comprising a transparent distal dielectric 278 made at least partially of silicone and conductive wires 276. Such an ablation device 110 may be used in any suitable method and device embodiments disclosed herein including, but not limited to the embodiments shown in FIGS. 2A and 5A. It can be seen in FIG. 1S that the tether 114 is continuous with the remainder of ablation device 110. Further, the tether is connected to the distal end of antenna 112.

Electrodes 118 and wires 276 may or may not be physically located on the device that comprises antenna 112. For example, electrodes 118 and wires 276 may be located on a separate device that is located around antenna 112. FIG. 1T shows an antenna similar to the antenna in FIG. 1A being slidably introduced through the lumen of a functional tube. In FIG. 1T, functional tube 188 comprises four equally spaced electrodes 118. Each electrode 118 is connected to a conductive wire 276. The four conductive wires 276 are arranged on functional tube 188 such that every curved segment of the conductive wires 276 is non-parallel to the axis of the functional tube 188. When antenna 112 is located within functional tube 188 such that antenna 112 is enclosed within electrodes 118, both electrodes 118 and a portion of conductive wires 276 are exposed to the microwave field generated by antenna 112. However, as discussed elsewhere in the specification, the novel arrangement of electrodes 118 and conductive wires 276 will not adversely affect the safety and efficacy of a procedure performed using antenna 112. During a method, the relative positions of antenna 112 and functional tube 188 may be changed by a user. In one embodiment, functional tube 188 comprises a steering or deflecting mechanism. The embodiment in FIG. 1T differs from the embodiment in FIG. 1B. In FIG. 1B, electrodes 118 and wire 276 are attached to antenna 112 or embedded inside antenna dielectric 266 of antenna 112. In FIG. 1T, electrodes 118 and wire 276 are slidable relative to antenna 112 and are not fixed to antenna 112.

Non-interfering designs of electrodes 118 and conductive wires 276 will be further illustrated in FIGS. 1U-1W. FIG. 1U shows a side view of a simulated SAR profile generated by a 915 MHz monopole antenna. FIG. 1V shows a side view of a simulated SAR profile generated by a monopole antenna similar to the antenna of FIG. 1U but having four extra electrodes spaced at regular intervals along the length of the antenna. The conductive wires 276 in this embodiment are arranged in a non-interfering arrangement as shown in FIG. 1B. FIG. 1V demonstrates that the SAR profile generated by this device embodiment having four electrodes 118 is similar to the SAR profile in FIG. 1U. Thus, the unique arrangement of electrodes 118 and wires 276 causes a clinically insignificant distortion of the microwave field generated by antenna 112. Further, the unique arrangement of electrodes 118 and wires 276 causes a clinically insignificant distortion of the thermal profile of the heat generated by antenna 112. In this simulation, four conductive wires 276 were combined into a bundle of wires. The SAR profiles in FIGS. 1V and 1U are substantially radially symmetric. Further, the SAR profiles in FIGS. 1V and 1U are substantially bilaterally symmetric on both sides of an imaginary plane oriented perpendicularly to antenna 112 and passing roughly through the proximal end of antenna 112. FIG. 1W shows a side view of a simulated SAR profile generated by a monopole antenna similar to the antenna of FIG. 1U but having four extra electrodes spaced at regular intervals along the length of the antenna wherein each electrode is connected to a conductive wire. The conductive wires 276 in this embodiment are linear and arranged parallel to the axis of antenna 112. FIG. 1W demonstrates that the SAR profile generated by the device embodiment having four electrodes 118 is significantly different from the SAR profile in FIG. 1U. Thus, this arrangement of electrodes 118 and wires 276 has caused a clinically significant distortion of the microwave field generated by antenna 112. The microwave field is concentrated in a smaller zone located near the proximal end of antenna 112. In this simulation, four conductive wires 276 were combined into a bundle of wires. The SAR profile in FIG. 1W is not bilaterally symmetric on both sides of an imaginary plane oriented perpendicularly to antenna 112 and passing roughly through the proximal end of antenna 112. Thus the lesion formed by the microwave field in FIG. 1W will be smaller than the lesion in FIG. 1V. A comparison of the SAR profiles in FIGS. 1W and 1V illustrate the usefulness of this unique arrangement of electrodes 118 and wires 276.

Further, the unique arrangement of electrodes 118 and wires 276 does not negatively affect the simulated return loss of a device with a monopole antenna. A simulated return loss of −14.90 was obtained at 915 MHz for a device with a monopole antenna of FIG. 1U. A simulated return loss of −19.52 was obtained at 915 MHz for a device with a monopole antenna and electrodes and electrode wire arrangement of FIG. 1V. This is actually a significant improvement over the return loss of the device of FIG. 1U. A simulated higher return loss of −12.86 was obtained at 915 MHz for a device with a monopole antenna and electrodes and electrode wire arrangement of FIG. 1W. Thus, the electrode and electrode wire arrangement of FIG. 1W has negatively affected the simulated return loss of a device with a monopole antenna.

FIGS. 2A-2D show various configurations of a flexible linear microwave antenna deployed in target regions of varying shape. An antenna 112 such as an antenna disclosed in one or more of US 2010/0137857, US 2011/0004205 and related patent applications (the entire disclosures of which are incorporated herein by reference) and a target region surface such as an external or internal anatomical surface are used only as examples to illustrate the general device and method embodiment of treating target regions of varying shape. In FIG. 2A, the surface of the target region is flat and thus antenna 112 is in a relatively straight configuration. FIGS. 2B-2D show antenna 112 deployed in locations with a single acute curve, a single less-acute curve and double curves respectively with a consequent change in the antenna 112 configuration. The shape or configuration change of antenna 112 is due to the change in shape of both radiating element 262 and shaping element 264 relative to an antenna axis 280 defined as a linear axis parallel to and emerging from the distal end of the transmission line 250. For example, in FIG. 2A, axes of both radiating element 262 and shaping element 264 are linear and in FIG. 2B, axes of both radiating element 262 and shaping element 264 are non-linear. Due to this shape change, there is a change in the interaction of the microwave energy emitted by the radiating element 262 relative to one or more of: radiating element 262, surrounding medium, the distal region of shielding element 256, antenna dielectrics 266 (if any), and floating conductors in the vicinity of radiating member 262 (if any). This in turn leads to a change in the electrical length of antenna 112 as described earlier. The change in the electrical length of antenna 112 and subsequent change in antenna impedance is detected by a change in the returned power (RP). The change in RP may be used to perform a variety of actions as disclosed elsewhere in this specification including, but not limited to: determining the configuration of antenna 112, determining the shape of the target region, determining the contour of a target region, determining the end-point of a procedure, etc.

In the antenna 112 of FIGS. 2A-2D a shaping element 264 is used to improve the performance of a helical antenna. The resultant antenna can be used to create a uniform zone of energy delivery along the length of the antenna 112 without adversely affecting material(s) surrounding the transmission line. In the antenna 112 of FIGS. 2A-2D, microwave ablation device 100 comprises a transmission line 250 such as a coaxial cable. An antenna 112 is connected to the distal end of coaxial cable 250. In the embodiment shown, the width of antenna 112 is substantially the same as the width of the coaxial cable 250. Microwave ablation device 100 divided into a first zone Z1 and a second zone Z2 (similar to the embodiment in FIG. 1A) by an imaginary transition line 254. First zone Z1 is proximal to second zone Z2. Transition line 254 is defined by the distal end of coaxial cable 250 and is substantially perpendicular to the axis of coaxial cable 250 at the distal end of coaxial cable 250. The distal region of coaxial cable 250 lies entirely within first zone Z1 and antenna 112 lies entirely within second zone Z2. In one embodiment, a single microwave signal is fed to antenna 112 through coaxial cable 250. When microwave energy is delivered to antenna 112 though coaxial cable 250, antenna 112 generates a microwave field. The near and/or far field of the microwave field generated by antenna 112 may be delivered to the target material. Antenna 112 comprises a radiating element 262 and a shaping element 264. In one embodiment, radiating element 262 is a continuation of the inner conductor 258 of coaxial cable 250. Shaping element 264 shapes the microwave field emitted by radiating element 262. Shaping member 264 is located distal to the distal end of coaxial cable 250 (in zone Z2). In one embodiment, shaping element 264 is made of an electrically conductive material e.g. a metal or a conductive polymer and is electrically connected to a region of outer conductor 256 of coaxial cable 250. In an alternate embodiment, a conductive shaping element 264 is electrically isolated from outer conductor 256. In this embodiment, shaping element 264 functions as a passive radiator or parasitic element of antenna 112. Shaping element 264 in this electrically isolated embodiment absorbs microwaves radiated from radiating element 262 and re-radiates microwaves. Referring back to FIG. 2A, it should be noted that there is no direct electrical conduction between radiating element 262 and shaping element 264. When microwave energy is delivered through coaxial cable 250 to antenna 112, a first microwave field is emitted by radiating element 262. This first microwave field is a normal mode microwave field of a small diameter (antenna diameter D is much less than microwave wavelength) helical antenna. The first microwave field interacts with shaping element 264. This interaction induces a leakage current on shaping element 264. The leakage current in turn creates a second microwave field. The second microwave field is an elongated, axial mode microwave field due to the elongate shape of shaping element 264. The first microwave field and the second microwave field together combine to produce a unique shaped microwave field of antenna 112 that is more useful that the unshaped microwave field generated by an antenna 112 comprising only radiating element 262. Thus the original microwave field is redistributed by the design of shaping element 264.

Further, the specific design of shaping element 264 may be used to improve the power deposition of an antenna 112 comprising radiating element 262. Shaping element 264 may be made of one or more non-insulated or insulated elements. Examples of such elements include, but are not limited to: straight or curved segments of metallic elements, elements with a circular or oval shape, elements with a polygonal shape (e.g. triangular, square, rectangular, pentagonal, etc.), multiple elements joined together by an electrically conducting joint(s), multiple elements joined together by a non-electrically conducting joint(s), elements with multiple curves, symmetrically arranged segments of elements, non-symmetrically arranged segments of elements, elements comprising outer coatings or layers of non-conducting materials, etc.

In the embodiment of FIG. 2A, the surface of radiating element 262 is enclosed within one or more layers of dielectric materials. The thickness and type of dielectric material along the length of radiating element 262 is engineered to optimize the microwave field shape. Thus one or more dielectric materials covering radiating element 262 may also be used as non-conducting shaping elements to shape the microwave field. The one or more dielectric materials covering radiating element 262 shape the microwave field by changing the local dielectric environment in the region adjacent to radiating element 262. In this embodiment, every portion of radiating element 262 is covered with some dielectric material such that no metallic surface of radiating element 262 is exposed to surrounding material. Thus, in this embodiment, radiating element 262 is electrically insulated from material. Thus, in this embodiment, radiating element 262 is able to transmit a microwave field into the surrounding material, but unable to conduct electricity to the surrounding material. Thus, in this embodiment, there is no electrical conduction and no conductive path between radiating element 262 and shaping element 264. Further, in this embodiment, there is no electrical conduction and no conductive path between radiating element 262 and the surrounding material. In one embodiment, the dielectric on a proximal portion of radiating element 262 is a continuation of the dielectric 260 of coaxial cable 250. The thickness of a dielectric on radiating element 262 may vary along the length of radiating element 262. Further, the cross section of a dielectric on radiating element 262 may not be radially symmetric.

In the embodiment of FIG. 2A, radiating element 262 is non-linear and is made of a helically arranged length of a metallic conductor. The helix may be symmetric with a constant pitch and a constant diameter along the length of the helix. In one embodiment, the straightened length of the conductor used for constructing radiating element 262 is about three quarters of the effective wavelength at 915 MHz. In alternate embodiments, this length may be an odd multiple of one quarter of the effective wavelength at one of: 433 MHz ISM band, 915 MHz ISM band, 2.45 GHz ISM band and 5.8 GHz ISM band. Although in FIG. 2A, radiating element 262 has about 19 turns, embodiments of ablation devices 100 may be constructed wherein radiating element 262 has about 1 to 30 turns. The pitch of a helical radiating element 262 may range between 0.3 mm and 20 mm. Radiating element 262 may be made from a metallic element or alloy selected from the group comprising Nitinol, stainless steel or copper. Any of the radiating elements 262 disclosed herein may comprise a plating of a conducting metal such as silver or gold on the outer surface of radiating element 262. The metallic conductor used for constructing radiating element 262 may have a round, oval, rectangular or square cross section. In one embodiment, the metallic conductor used for constructing radiating element 262 has a round cross section with a diameter of 0.5 mm+/−0.4 mm. In another embodiment, the metallic conductor used for constructing radiating element 262 has a rectangular cross section with cross sectional dimensions of 10 mm+/−9.5 mm by 0.5 mm+/−0.4 mm. In another embodiment of a radiating element with a rectangular cross section, the cross sectional dimensions are 1 mm+/−0.3 mm by 0.1 mm+/−0.05 mm. In an alternate embodiment, radiating element 262 is made of a length of a metallic conductor that is arranged in a substantially two dimensional configuration. For example, the length of a metallic conductor may be arranged in a substantially wavy or zigzag or serpentine configuration. In the embodiment in FIG. 2A, radiating element 262 is arranged symmetrically around shaping element 264 and partially or fully encloses shaping element 264. Shaping element 264 may be made of a linear or helical length of a metallic conductor. The outer diameter of shaping element 264 may be uniform or may be non-uniform along the length of antenna 112. In the embodiment shown in FIG. 2A, shaping element 264 is made of a substantially linear length of a metallic conductor. Shaping element 264 may be made from a metallic element or alloy selected from the group comprising Nitinol, stainless steel or copper. Shaping element 264 may comprise a plating of a conducting metal such as Ag or Au on the outer surface of shaping element 264. The metallic conductor used for constructing shaping element 264 may have a round, oval, rectangular or square cross section. In one embodiment, the metallic conductor used for constructing shaping element 264 has a round cross section with a diameter of 0.5 mm+/−0.3 mm. In another embodiment, the metallic conductor used for constructing shaping element 264 has a rectangular cross section with dimensions of 0.5 mm+/−0.3 mm by 0.5 mm+/−0.3 mm. Antenna 112 further comprises one or more antenna dielectrics 266 between radiating element 262 and shaping element 264. In one embodiment, antenna dielectric 266 is sufficiently flexible to create a flexible antenna 112. The flexibility of antenna 112 allows antenna 112 to bend from a substantially straight, linear configuration to a substantially non-linear configuration and vice-versa during use. The flexibility of antenna 112 also allows antenna 112 to bend relative to the distal end of the transmission line during use. This in turn allows a user to introduce antenna 112 to the target location through tortuous or non-linear introduction paths. In one embodiment, antenna dielectric 266 is sufficiently stiff to create a sufficiently stiff antenna 112. The stiffness of antenna 112 prevents antenna 112 from bending during use. This in turn enables the user to use antenna 112 to puncture or penetrate through a target material. Such embodiments of antenna 112 may be used for ablating solid volumes of materials. Examples of dielectrics that can be used between radiating element 262 and shaping element 264 include, but are not limited to EPTFE, PTFE, FEP and other floropolymers, Silicone, Air, PEEK, polyimides, natural or artificial rubbers and combinations thereof. Additionally the entire antenna 112 may be covered or encapsulated in a dielectric. Examples of dielectrics that can be used to cover or encapsulate antenna 112 include, but are not limited to EPTFE, PTFE, FEP and other floropolymers, Silicone, PEEK, polyimides, natural or artificial rubbers and combinations thereof. Antenna dielectric 266 may comprise one or more layers of such dielectrics. The dielectric used to cover or encapsulate antenna 112 may be porous or non-porous. In FIG. 2A, the length of antenna 112 may be between 5 mm and 80 mm and the width of antenna 112 is between 1 mm and 40 mm. In one particular embodiment, antenna 112 has a length of 45 mm+/−7 mm and a width of 2 mm+/−0.5 mm. Radiating element 262 is electrically connected to inner conductor 258 of coaxial cable 250. This may be done for example, by soldering or resistance welding radiating element 262 to inner conductor 258. Radiating element 262 may be a continuation of inner conductor 258 of coaxial cable 250. Shaping element 264 is electrically connected to outer conductor 256 of coaxial cable 250. This may be done for example, by soldering or resistance welding shaping element 264 to outer conductor 256. Antenna 112 may be floppy, flexible or substantially rigid. Antenna 112 may be malleable or have shape memory or elastic or super-elastic properties. The distal end of antenna 112 may be soft or atraumatic. Antenna 112 may be designed such that the length of antenna 112 is adjustable. For example, length of antenna 112 may be increased or reduced to increase or reduce the length of an ablation zone. In this embodiment, shaping element 264 may have a helical or substantially wavy or zigzag or serpentine configuration. The length of antenna 112 may be increased or reduced during and/or before a procedure. In one embodiment, the length and/or the diameter of the ablation zone is changed by one or more of: changing the length of radiating element 262, changing the length of shaping element 264, changing the shape of radiating element 262, changing the shape of shaping element 264 and changing the relative positions of radiating element 262 and shaping element 264. Any of the embodiments of changing the shape or an antenna 112 disclosed herein may lead to a change in the effective length of the antenna 112 which in turns leads to a change in the antenna impedance. This may be detected by measuring RP at one or more times during use. In one embodiment, one or both of radiating element 262 and shaping element 264 are a part of a flexible circuit and are manufactured using commonly known techniques for manufacturing flexible circuits.

In the embodiment of FIG. 2A, the shape of radiating element 262 is different from the shape of shaping element 264. Further in the embodiment in FIG. 2A, radiating element 262 is non-linear. Further in the embodiment in FIG. 2A, shaping element 264 is substantially linear. However radiating element 262 and shaping element 264 are generally oriented such that their axes are parallel to each other. Alternate embodiments of antenna 112 may be designed wherein radiating element 262 is substantially linear. Alternate embodiments of antenna 112 may be designed wherein shaping element 264 is substantially non-linear. Alternate embodiments of antenna 112 may be designed wherein radiating element 262 and shaping element 264 are generally oriented such that their axes are not parallel.

Although in the embodiment in FIG. 2A shaping element 264 is connected to the distal end of coaxial cable 250, other embodiments of antenna 112 may be designed wherein shaping element 264 is connected to coaxial cable 250 at a region other than the distal end of coaxial cable 250. For example, in one alternate embodiment, shaping element 264 is metallic and is electrically connected to a region of outer conductor 256 of coaxial cable 250 proximal to the distal end of the coaxial cable 250.

In FIG. 2A, since radiating element 262 is in electrical contact with inner conductor 258, there is a first electrically conductive path extending from inner conductor 258 till the distal end of radiating element 262. In the embodiments wherein shaping element 264 is made of a conductive material and is electrically connected to outer conductor 256 of coaxial cable 250, there is a second electrically conductive path extending from outer conductor 256 till the distal end of shaping element 264. In such embodiments, even though there are two conductive paths that extend from first zone Z1 to the second zone Z2, the designs, materials and the microwave properties of the two conductive paths may be significantly different in first zone Z1 and second zone Z2. In first zone Z1, outer conductor 256 of coaxial cable 250 is located symmetrically around inner conductor 258 and at a constant distance from inner conductor 258. However, in second zone Z2, radiating element 262 is located symmetrically around shaping element 264 and at a constant distance from shaping element 264. In first zone Z1, outer conductor 256 of coaxial cable 250 always acts as a shield for the microwave field in first zone Z1 whereas in second zone Z2, shaping element 264 may or may not act as a shield for the microwave field in second zone Z2.

In any of the embodiments herein, radiating element 262 may be a continuation of inner conductor 258 of a coaxial cable 250. In another embodiment, radiating element 262 is length of a conductor attached to inner conductor 258. In one embodiment, the proximal end of radiating element 262 is electrically connected to the distal end of inner conductor 258. In one embodiment, the proximal end of radiating element 262 is soldered to inner conductor 258. In another embodiment, the proximal end of radiating element 262 is laser welded to inner conductor 258. The proximal end of radiating element 262 may be electrically connected to inner conductor 258 in various configurations including, but not limited to lap joint and butt joint. The proximal end of shaping element 264 is electrically connected to a region of outer conductor 256. In one embodiment, the proximal end of shaping element 264 is electrically connected to the distal end of outer conductor 256. In one embodiment, the proximal end of shaping element 264 is soldered to outer conductor 256. In another embodiment, the proximal end of shaping element 264 is laser welded to outer conductor 256. The proximal end of shaping element 264 may be electrically connected to outer conductor 256 in various configurations including, but not limited to lap joint and butt joint.

The SAR profile generated by the device embodiment of FIG. 2A is substantially radially symmetric around antenna 112 and circumferentially and volumetrically envelops entire antenna 112. This entire circumferentially and volumetrically enveloping microwave field around antenna 112 can be delivered to the target material. Further, the microwave field generated by antenna 112 of FIG. 2A is substantially restricted to second zone Z2. There is an insignificant amount of the microwave field in first zone Z1 containing coaxial cable 250. Thus, there is negligible backward coupling between the microwave field and the distal portion of coaxial cable 250. This in turn reduces the risk of ablating material proximal to the distal end of coaxial cable 250. Further, the microwave field is substantially uniform along the length of antenna 112 as compared to a comparable monopole antenna. Embodiments of linear antenna 112 designed to operate at 915 MHz and other microwave frequencies may be designed that can create uniform, symmetrical, continuous, linear or volumetric lesions with a lesion length greater than 35 mm.

In alternate embodiments, the SAR profile may be designed to be substantially non-uniform along the length of a linear antenna 112. For example, an antenna 112 may be designed to have a SAR profile that is wider and/or stronger at the center of antenna 112 and is less strong at the ends of antenna 112. In order to achieve this, one or more design parameters of antenna 112 in FIG. 2A may be modified. Examples of such modifications include, but are not limited to: adding of one or more additional conductive shaping elements 264; varying the width and/or the cross section shape of shaping element 264 and/or radiating element 262 along the length of antenna 112; varying the pitch of helical radiating element 262 and/or helical shaping element 264 along the length of antenna 112; varying the thickness, type and other design parameters of one or more antenna dielectrics 266, etc.

Antenna 112 in FIG. 2A has several advantages over a comparable monopole antenna. In systems comprising a monopole antenna, there is a region of concentrated microwave field or a “hot spot” near the distal end of the transmission line (e.g. a coaxial cable) or at the proximal end of the monopole antenna. About half of the microwave field in such systems is present in first zone Z1. Thus, there is a significant amount of microwave field present in first zone Z1. Thus, there is a high risk of ablating material proximal to the distal end of coaxial cable 250. The presence of a significant amount of microwave field in first zone Z1 is due to undesirable coupling between the microwave field and the outer conductor of the coaxial cable or other transmission line. This undesirable coupling can also cause backward heating of coaxial cable 250 that may lead to collateral damage of material.

In several of the embodiments herein, shaping element 264 plays a critical role in shaping the microwave field generated by antenna 112. In the embodiment of FIG. 2A, the nearest conductive path to the microwave field emitted by radiating element 262 is provided by the conductive shaping element 264 instead of the shielding element of the distal region of the transmission line 250. The presence of shaping element 264 prevents the microwave field from coupling to the distal region of the transmission line 250. Virtually none of the microwave field will be located around the distal region of transmission line 250. Further, since a vast majority of the emitted microwave field is deposited in zone Z2, the power deposition of antenna 250 is improved. Virtually no portion of the field is wasted in zone Z1. Further, the shaping element 264 in antenna 112 of FIG. 2A improves the matching and reduces the return loss.

Any of the shaping elements 264 herein may be used to provide an additional resonance point in the frequency spectrum. This in turn may be used to increase the frequency range (bandwidth) over which antenna 112 delivers an acceptable performance. For example, the design of shaping element 264 in FIG. 2A improves the frequency range over which important performance parameters are acceptable. Thus, a larger frequency range (bandwidth) is available over which antenna 112 delivers an acceptable performance. This in turn allows for a design of antenna 112 wherein minor deformations of antenna 112 during use or due to minor manufacturing variations do not significantly affect the performance of antenna 112, but can be detected using the present invention.

In one particular embodiment of antenna 112 of FIG. 2A, dielectric 266 is transparent and flexible. The linear length of antenna 112 from the distal end of coaxial cable 250 till the distal end of radiating element 262 is about 4.5+/−0.5 cm. Alternate embodiments of antenna 112 may be designed with a linear length ranging from 2.5-5.5 cm. In the particular embodiment, the outer diameter of antenna 112 is about 2 mm. Alternate embodiments of antenna 112 may be designed with an outer diameter ranging from 1.5-4 mm.

In one method embodiment, antenna 112 is used to deliver microwave energy to multiple target regions by repositioning antenna 112. The shape of antenna 112 may or may not be the same at these multiple regions. Antenna 112 may be bent during use without adversely affecting its microwave field shape.

In one method embodiment, a radiating element 262 and a shaping element 264 of an antenna 112 are placed on opposite sides of a target material. Shaping element 264 shapes the microwave field emitted by radiating element 262 such that the microwave field is concentrated in the region between radiating element 262 and shaping element 264. This concentrated microwave field in the region between radiating element 262 and shaping element 264 is used to achieve the desired effect in the material.

In one method embodiment, a target material is located between an antenna 112 and a microwave shield or reflector. Thereafter, microwave energy is delivered to treat the target material.

Any of the embodiments of RP measurement disclosed herein may be used to manually or automatically alter the energy delivery settings during the microwave energy delivery. Examples of such energy delivery settings include, but are not limited to: time limit of the microwave energy delivery, the average power, and the pulse width, height or frequency if the microwave energy is delivered in discrete pulses (i.e. altering the duty cycle).

Any of the embodiments herein may use RP measurements for automatic or manual termination of energy delivery. Such method embodiments can use any of the antennas 112 disclosed herein and in one or more of US 2010/0137857, US 2011/0004205, US 2010/0121319, US 2010/0125269 and related patent applications, the entire disclosure of which are incorporated herein by reference.

As microwave energy is delivered to a target material, one or more properties of the target material such as moisture content, temperature, impedance, a physical dimension, permittivity, dielectric constant, loss tangent, resistivity, hardness, and friability, and other properties disclosed herein progressively change. This changes the microwave interaction of antenna 112 with the surrounding medium which in turn changes the effective length of antenna 112 as described earlier. Thus the impedance matching of an antenna with the target tissue will progressively change as the microwave energy delivery progresses leading to progressively varying RP. This degree of RP variation will be dependent on the degree of microwave-induced change of the target material. Thus the present invention can be used to determine the degree of change in the target material due to the effect of the microwave energy. Further, the present invention may also be automatically shut off the energy delivery after creating a desired microwave-induced change (e.g. by a set RP limit) or convey to the user the changed RP indicating the creation of the desired material change.

In one such embodiment of microwave ablation antennas, as microwave energy is delivered to a target tissue, the tissue gets ablated and properties of tissue such as moisture content and other properties disclosed herein progressively change. This changes the microwave interaction of antenna 112 with the surrounding medium which in turn changes the effective length of antenna 112 as described earlier. Thus the matching of an antenna with the target tissue will progressively change as the ablation progresses leading to progressively varying RP. This degree of RP variation will be dependent on the degree of ablation of the tissue. Thus the present invention can be used to determine the degree of ablation. Further, the present invention may also be automatically shut off the energy delivery after creating a desired ablation (e.g. by a set RP limit of 25+/−10%) or to indicate to the user the changed RP indicating the creation of the desired ablation.

In one such embodiment of endometrial ablation, as the ablation progresses, the RP progressively increases. The ablation is allowed to progress till the RP reaches as set RP limit (e.g. a set RP limit of 25+/−10%) or a time limit after which the system automatically terminates energy delivery. This allows the full thickness of the endometrium to be ablated while preventing excessive delivery of microwave energy which might compromise procedure safety.

FIG. 2E shows an embodiment of an antenna similar to the antenna in FIGS. 2A-2D comprising a radiating element and a shaping element connected to each other by one or more flexible dielectric attachments. The attachments 266 shown in FIG. 2E are made of a dielectric material and thus called antenna dielectrics 266 in this specification. Antenna dielectric 266 allows a greater relative motion between radiating element 262 and shaping element 264 than in the antenna 112 in FIG. 2A wherein both radiating element 262 and shaping element 264 are embedded in an antenna dielectric 266. Antenna dielectrics 266 may be made of one or more of the following: elements with a spring action; flexible elastic elements; flexible non-elastic elements; straight or curved segments of dielectric materials; etc. In one embodiment, radiating element 262 is flexible and antenna 112 self-expands in diameter in a mechanically unconstrained environment. In a mechanically constrained environment, antenna 112 self-expands to the maximum extent possible. The antenna impedance in the constrained configuration and the non-constrained configuration are different.

FIGS. 2F, 2G and 2H show three configurations of antenna 112 of FIG. 2E in a constrained configuration, a less constrained configuration and a least constrained configuration respectively. The configuration change is defined as a change in the shape of one or both of: radiating element 262 and shaping element 264 relative to an antenna axis 280 defined as a linear axis parallel to and emerging from the distal end of the transmission line 250. For example, in FIGS. 2G and 2H, shaping element 264 is in an axially expanded configuration or shape and in FIG. 2F, shaping element 264 is in an axially compressed configuration or shape. In FIG. 2F, antenna 112 is deployed in a target region of a smaller dimension. The target region is of a sufficient stiffness so that it does not get deformed when antenna 112 is deployed in the target region. This causes antenna 112 to be deployed in a more constrained configuration in the target region of a smaller dimension in FIG. 2F as compared to the target region of a greater dimension in FIG. 2G. The antenna configurations in constrained and un-constrained configurations may be different due to one or more of: change in the shape of radiating element 262, change in the shape of shaping element 262, change in the position and/or shape of antenna dielectrics 266, and change in the length of antenna 112. Specifically, in the embodiments shown in FIGS. 2F and 2G, the shape of radiating element 262 has been changed along with the shape and/or the position of antenna dielectrics 266. This leads to a change in the position of radiating element 262 relative to shaping element 264, surrounding medium, the distal region of shielding element 256, antennas dielectrics 266 (if any), and floating conductors (if any) in the vicinity of radiating member 262. This in turn leads to a change in the electrical length of antenna 112 due to the change in the interaction of the microwave energy emitted by the radiating element 262 with shaping element 264. The change in the electrical length of antenna 112 and the corresponding change in the antenna impedance are detected by a change in the returned power (RP). The change in RP may be used to perform a variety of actions as disclosed elsewhere in this specification.

Any of the microwave devices disclosed herein may use a variety of feedback mechanisms during their use. In one such embodiment, one or more returned power measurements are used to take a variety of decisions before, during, or after an energy delivery procedure. Examples of such returned power measurements and decisions are disclosed in US patent application publication no. 2014/0190960, the entire disclosure of which is incorporated herein by reference.

In the present specification, RP is defined as one or more primary or derived parameters or combination thereof of the microwave energy returned towards the microwave energy source (e.g. a generator) at one or more times during a procedure as described below.

In one embodiment, RP is calculated as a fraction by dividing the returned power (e.g. as measured by a power detector) by the forward power (e.g. as measured by a power detector). i.e. RP=(Returned Power)/(Forward Power). This returned power fraction may then be used to take one or more user level or system level decisions.

In one embodiment, the magnitude of the returned power is measured (e.g. in Watts). E.g. the average returned power may be measured. This returned power magnitude may then be used to take appropriate user level or system level decisions.

The returned power may be measured one or more times before the start of a microwave energy based procedure and/or during the microwave energy based procedure and/or after the end of the microwave energy based procedure. The returned power may be measured continuously during one or more periods of time or may be measured intermittently.

In one embodiment, a graph of returned power with a second parameter is calculated and/or displayed on a screen. Examples of such second parameters include, but are not limited to: time, position of one or more components of medical system 100 within a target material, and various deployment configurations of an antenna. In one such embodiment, a graph of returned power with time is recorded and/or displayed on a screen. In another such embodiment, a graph of returned power with position of an antenna is recorded and/or displayed on a screen.

In embodiments wherein the change in returned power with a second parameter is calculated and/or displayed, one or more data processing steps may be performed on the returned power measurements. Examples of such data processing steps include, but are not limited to: calculating the trend of the returned power with the second parameter, calculating the first derivative i.e. the slope of the returned power, calculating the second or higher derivatives of the returned power, and calculating the area-under-the-curve of the returned power.

In one embodiment, the measured returned power is adjusted for expected microwave energy losses. Examples of such losses include, but are not limited to: losses encountered within a generator after the forward power is measured by a power meter, losses encountered within a transmission line 250, losses encountered within shaft of device 110, losses encountered within antenna 112, reflections occurring at one or more interfaces from the point forward power is measured till the interface between the antenna and the shaft of device 110, expected measurement errors while measuring forward power and/or returned power, etc. The adjusted returned power is then used to take appropriate user level or system level decisions.

In one embodiment, the expected microwave energy losses are estimated after positioning antenna 112 inside a test material (e.g. saline or water). The test material may be chosen such that it simulates actual target material. The returned power measurements made after delivering microwave energy inside the test material can then be correlated with the expected microwave energy losses when antenna 112 is positioned inside the actual target material. In one embodiment, the test material is a microwave energy sink physically located inside the microwave generator and the transmission line 250 is directly connected to a port connected to the test material to test one or more of: a generator, transmission line 250 and any system connections as per the embodiments disclosed elsewhere in this specification.

In one embodiment, a lower level of microwave energy called test power is delivered and the corresponding RP is measured. This test power is low enough to not cause any appreciable microwave energy based changes in the target material. The returned power from the test power is used to take appropriate user level or system level decisions. In one such embodiment, the delivery and measurement of test power is done (one or more times) before and/or after one or more periods of typical microwave energy delivery. In another embodiment, the test power is delivered such that the total energy delivered is insignificant e.g. <1 J. In another embodiment, the delivery and measurement of test power is done (one or more times) intermittently in between periods of typical microwave energy delivery. In one embodiment, the test power is delivered as a pulse of a small pulse width (e.g. <0.1 s). In one embodiment, the magnitude of the test power is gradually increased or ramped up over a period of time (e.g. 3 s).

One or more statistical analyses of the returned power measurements may be made. Examples of simpler statistical analysis of RP measurements include, but are not limited to estimating one or more of: maximum, minimum, average, median, mode, standard deviation, etc. of the one or more measured values. The statistical analysis may be performed by comparing the actual RP value with previously stored or separately generated RP values. One or more trendlines may be plotted for one or more RP measurements. Examples of types of such trendlines include, but are not limited to: linear, logarithmic, power, polynomial, exponential, and moving average. The data from one or more RP measurements may be filtered using one or more filtering criteria. Examples of more complex statistical analysis of RP measurements include, but are not limited to a) determining the R (correlation coefficient) value to measure of the strength of linear dependence between RP measurements and a second parameter, b) R.exp.2 (coefficient of determination) to predict outcomes on the basis of other related information, etc.

In one embodiment, one or more additional parameters such as data obtained from one or more sensors are used in addition to data from one or more RP measurements to take one or more decisions. Examples of such additional parameters include, but are not limited to: temperature measurements, pressure measurements, electrical measurements at one or more regions, direct or machine aided visualization of the target material or one or more components of the system, data from a visual display, pre-existing data, etc.

One or more decision levels or limits may be used to take user level or system level decisions. In one embodiment, one or more decision levels or limits (e.g. one or more of 20%, 25%, 30%, 45%, etc.) are pre-programmed in the system software and/or hardware.

In another embodiment, one or more decision levels or limits are set either by a user or by the system based on defined criteria e.g. initial RP measurements, properties of the target material, etc. In one embodiment, the user inserts device 110 into the target material. Thereafter, a test microwave power is delivered and the returned power is measured. This returned power level is then used by the system and/or the user to set the one or more decision levels or limits for taking user level or system level decisions.

In another embodiment, one or more decision levels or limits are based on one or more initial measurements of RP. For example, a first measurement is made before an energy delivery cycle to get an initial RP level. This initial RP level is then used to determine one or more decision levels or limits. In one such embodiment, the system automatically prevents energy delivery energy delivery if the initial RP level is above a threshold.

In another embodiment, one or more decision levels or limits are based on the size of the target material. E.g. the size of the object or an anatomical organ to be treated. In one such embodiment, a higher RP limit is used for treating a larger target material to allow more energy delivery to the larger anatomy.

In another embodiment, one or more decision levels or limits are based on the shape of the target material.

In another embodiment, one or more decision levels or limits are based on the desired outcome of the energy delivery. In one such embodiment of endometrial ablation, a first set of one or more decision levels or limits are used when reduction in menstruation rather than amenorrhea is the desired outcome and a second set of one or more decision levels or limits are used when amenorrhea rather than reduction in menstruation is the desired outcome. In one such embodiment, one or more decision levels or limits and the energy delivery settings are used to automatically limit ablation depth to 1-4 mm beyond the endo-myometrial junction. In one such embodiment, the energy delivery settings are set to deliver a generator output dose ranging between 2,500-5,000 J. In one such embodiment, one or more decision levels or limits and the energy delivery settings are used to automatically limit ablation depth to 3-8 mm into the wall of a renal artery. In one such embodiment, the energy delivery settings are set to deliver a generator output dose ranging between 300-5,000 J.

In another embodiment, one or more decision levels or limits are based on the desired clinical condition to be treated. In one such embodiment of uterine ablation, a first set of one or more decision levels or limits are used for treating adenomyosis and a second set of one or more decision levels or limits are used for treating hyperplasia.

In another embodiment, one or more decision levels or limits are based on the desired depth of penetration of the microwave energy or the desired size of the thermal zone created using the microwave energy.

In another medical embodiment, one or more decision levels or limits are based on the pre-procedure measurements of one or more regions of the anatomy. Such pre-procedure measurements may be made using a variety of imaging tools (e.g. endoscopy, ultrasound imaging, direct mechanical measurement of one or more anatomical regions, etc.

In one embodiment, the energy delivery is adjusted in real time based on one or more or RP parameters and procedure feedback. Examples of RP parameters include, but are not limited to: total increase in RP, rate of increase of RP (e.g. unexpectedly sharp increase, unexpectedly slow increase, etc.) and other RP parameters as disclosed elsewhere in this specification. Examples of procedure feedback include, but are not limited to: pain experienced by the patient, imaging feedback about the progress of the procedure, change in a patient parameter, etc.

The following are embodiments of the timing of RP measurement(s). In one embodiment, RP is measured before or at the start of actual microwave energy delivery. The RP may be measured once or multiple times or continuously till a desired RP level is obtained. Thereafter, the delivery of power may be started.

In one embodiment, RP is measured during one or more device movements. The device may be subjected to one or more movements including, but not limited to:

A. adjusting the position of the device 110 and/or antenna 112 relative to a target material,

B. deploying and/or undeploying antenna 112 into the target,

C. turning or twisting the device 110 and/or antenna 112, and

D. engaging a steering or deflecting mechanism that steers or deflects device 110 and/or antenna 112.

In one embodiment, RP is measured during a procedure of delivering microwave power. The measurements may be continuous or discreet. In one embodiment, microwave energy is delivered discontinuously and the measurements are made during one or more of microwave “on” times and microwave “off” times.

Similarly, RP may be measured after a procedure of delivering microwave power. This may be used for example to obtain feedback about the device 100 and/or the procedure.

In one embodiment, RP is measured after altering antenna 112. In one such embodiment, the shape of antenna 112 is altered and the returned power is measured before and/or during and/or after changing the shape of antenna 112. The shape of the antenna 112 may be altered by one or more of: twisting the antenna 112, engaging a shape altering mechanism that alters the shape of antenna 112, and partially or completely deploying or undeploying antenna 112. The level and/or the change in the level of RP with the amount of alteration of antenna 112 may be measured and used to take further decisions.

In one embodiment, RP is measured after altering the local environment around antenna 112. In one such embodiment, a fluid (e.g. a liquid or gas) is introduced around one or more regions of antenna 112. The fluid environment may alter (e.g. improve or worsen) the matching between antenna 112 and the surrounding target material. The level and/or the change in the RP with the amount of alteration of the local environment around antenna 112 may be measured and used to take further decisions.

Any information about the RP in any of the embodiments disclosed herein may be used to alert the user or may be fed back into the generator to take one or more decision. The user can be alerted by one or more of: sounding an alarm, displaying the RP measurement(s), sending information to another device, and displaying one or more derived parameters of the RP such as a graph of RP against a secondary parameter.

The target region in FIG. 2H has the same dimension as that in FIG. 2F. However, in this embodiment, the target region is of a sufficient flexibility so that it gets deformed when antenna 112 is deployed in the target region. This causes the antenna 112 to be deployed in a less constrained configuration in FIG. 2H as compared to the embodiment in FIG. 2F. Comparing FIGS. 2F and 2H, the shape of radiating element 262 has been changed along with the shape and/or the position of antenna dielectrics 266. This leads to a change in the position of radiating element 262 relative to shaping element 264, surrounding medium, the distal region of shielding element 256, antennas dielectrics 266 (if any), and floating conductors (if any) in the vicinity of radiating member 262. This in turn leads to a change in the electrical length of antenna 112 due to the change in the interaction of the microwave energy emitted by the radiating element 262 with shaping element 264. The change in the electrical length of antenna 112 and the corresponding change in the antenna impedance are detected by a change in the returned power (RP). The change in RP may be used to perform a variety of actions as disclosed elsewhere in this specification. In one embodiment, the RP of a system comprising an antenna 112 of FIG. 2E increases as the degree of constraint of antenna 112 increases. The embodiments in FIGS. 2F-2H demonstrate that the RP value may be a function of both the mechanical and microwave properties of the target material. Any of the embodiments herein may use RP feedback to determining the mechanical and/or microwave properties of the target material.

In one such embodiment, antenna 112 has a higher RP when deployed in a more constrained configuration and a lower RP when deployed in a less constrained configuration. Thus, for the same generator setting, antenna 112 will deliver a greater energy dose in anatomical regions of larger size and a lesser energy dose in anatomical regions of smaller size. In another embodiment, the user may set the generator to deliver a lower energy dose if the RP is higher, since the high RP indicates that the antenna 112 is in a more constrained configuration in an anatomical region of smaller size.

Any of the medical system 100 disclosed herein may comprise a hollow sheath or arm 102. In the specification, the term “arm” is used to describe any elongate device that can be used to introduce and/or manipulate one or more medical components 110. Thus, arm can be an introducing catheter, a cannula, a tubular sheath, etc. Arm 102 may comprise a maneuverable distal portion 104 and a proximal portion 106, such that manipulation of proximal portion 106 of arm 102 permits articulation of maneuverable distal portion 104 of arm 102. A medical component 110 is mechanically coupled to arm 102 and is adjustably extendable from maneuverable distal portion 104 of arm 102. Medical component 110 is mechanically engaged to maneuverable distal portion 104 of arm 102, such that movement of maneuverable distal portion 104 alters the position and/or the orientation of medical component 110.

With reference to FIG. 2I, a first embodiment of the present invention comprising an elongate arm will be discussed. FIG. 2I depicts ablation system 100 as generally including a guide sheath 102 and a medical component 112 comprising a transmission medium or transmission line 250 ending in an ablating portion 112. Guide sheath 102 includes a flexible outer tube and an inner lumen which passes therethrough. The flexible outer tube may be made of any suitable material such as medical grade polyolefins, fluoropolymers, or polyvinylidene fluoride. For illustration purposes only, PEBAX® resins from Autochem of Germany can be used. Ablating portion 112 is adapted to transmit an ablative energy. Examples of ablative energies that can be used in the present invention include, but are not limited to RF energy, microwave energy, ultrasound energy, thermal energy, cryogenic energy and infrared energy. In an alternate embodiment, ablating portion 112 is adapted to transmit high energy particles. Examples of such high energy particles include, but are not limited to ionized particles, electrons, X-ray photons, ultraviolet photons, and gamma photons. In an alternate embodiment, ablating portion 112 is adapted to release an ablative chemical. In the particular embodiment disclosed in FIG. 1I, ablating portion 112 has a single ablating element. The ablating element is a microwave antenna selected from any of the antennas disclosed herein. The antenna 112 may be encased within a dielectric material, flexible polytetafluoroethylene (PTFE), often referred to by its trademark TEFLON®, or expanded PTFE (ePTFE) for example.

In the embodiment in FIG. 2I, guide sheath 102 is adapted to initially approach the target tissue with the plane of ablating portion 112 oriented parallel to the distal region of guide sheath 102. In the embodiment in FIG. 2J, guide sheath 102 is adapted to initially approach the target tissue substantially normal to the tissue surface, shown in a deflected orientation engaging the tissue surface. It is important to note that other approach angles and corresponding degrees of deflection are also contemplated and that the depicted orientation is for illustration purposes only. Moreover, the deflection functionality can be solely provided by the ablating portion 112 itself when advantageous, for example when guide sheath 102 does not includes a steering system to steer or direct its distal end.

The deflection of any of the sheaths 102 disclosed herein can be achieved through any suitable means. For example, sheath 102 can include a steering system, including one or more pull wires, which is adapted to form the desired deflection required to position the distal opening generally normal to the target tissue surface adjacent thereto. Alternatively, shape retaining materials can be used. For example, the distal portion of the guide sheath 102 can be formed from shape retaining materials, tubular structures made from polyethylene or including super-elastic metal such as Nitinol for example, and interfaced to the elongate member of the guide sheath 102 via a flexible portion or flexible joint, as discussed later herein. In any case, the ablation sheath 12 can be composed of any suitable flexible biocompatible material, such as PU Pellethane, TEFLON® or polyethylene, which, as stated above, is capable of shape retention once external forces acting upon the sheath 102 are removed.

It is important to note, while currently discussed in terms of positioning the distal opening of sheath 102 generally perpendicular to the target tissue surface, additional placements adjacent to or removed from the target tissue surface are also contemplated such as normal to the target tissue, as discussed in more detail below.

The transmission line of the embodiments in FIGS. 2I and 2J can be a suitable flexible coaxial cable of the desired size, having an outer conductor and an inner conductor separated by a dielectric material enclosed in an outer jacket. The outer jacket or dielectric material can be any suitable biocompatible material, such at PTFE. For illustration purposes only, the outer jacket may be constructed from solid, but flexible. PTFE, while the dielectric material may be constructed from expanded PTFE which is advantageous due to its increased flexibility and radial stability.

The inner conductor of the transmission line can be electrically coupled directly or indirectly to the ablation portion 112 through any suitable means such as soldering, brazing, ultrasonic welding or adhesive bonding. In other embodiments, ablation portion 112 can be formed from the inner conductor of the transmission line itself, the outer conductor and none, part, or all of the dielectric material surrounding the center conductor being removed, as desired. This is typically more difficult from a manufacturing standpoint but has the advantage of forming a more rugged connection between the antenna and the inner conductor. In other embodiments, it may be desirable to indirectly couple the antenna to the inner conductor through a passive component, such as a capacitor, an inductor or a stub tuner for example, in order to better adapt the antenna system for ablation of the specific biological target tissue.

The ablation portion 112 includes an electrically conductive material from which the electromagnetic energy is transmitted. For illustration purposes only, copper or silver-plated metal are well suited for transmission of such electromagnetic energy. While ablation portion 112 may be formed from a solid, but flexible, piece of electrically conductive material, ablation portion 112 may be formed from other suitable materials, polymers or other plastics or resins for example, the electrically conductive material being deposited at one or more locations along the length of structure, each location being electrically connected to the center conductor of the transmission line. Additionally, the ablation portion 112 can be a braided structure, the braided structure adapted to provide increased flexibility while preventing substantial signal loss.

The ablation portion 112 diameter can be any suitable size which allows for the transport of the ablation portion 112 to the target tissue site and transmission of electromagnetic energy thereto. Such diameters include the range from about 0.2 mm to about 4 mm, but can be larger in diameter if desired. The dielectric 260 may hold the ablation portion 112 a known distance away from the target tissue, a distance ranging from about 0.2 mm to about 4 mm. For operating frequencies disclosed herein, ablation portion 112 can be of any suitable length. For illustration purposes only, for an operating frequency of approximately 2.45 GHz the ablation portion 112 can be from about 12 mm to about 20 mm in length. Given other operating frequencies, longer lengths can be achieved.

As shown in FIGS. 2K and 2L, the ablation portion 112 is encased within the dielectric 260. While shown coaxial with the outer surface of the dielectric 260 material, antenna 112 may be positioned offset with respect to the longitudinal axis of the dielectric 260, closer to the target tissue surface for example. Alternatively, the dielectric may have a non-circular cross-sectional surface, the antenna located inline or offset with respect to the cross-sectional geometric center. The insulating dielectric 260 material is preferably a low-loss dielectric material which is relatively unaffected by microwave exposure, and thus capable of transmitting the electromagnetic energy therethrough. Moreover, the dielectric preferably has a low water absorption component such that it does not react by thermally heating due to direct exposure to the electromagnetic energy. With this in mind, the dielectric 260 may be formed from any suitable biocompatible materials, including, but not limited to, moldable PTFE or ePTFE, silicone, or polyethylene, polyimide, or other suitable material having similar qualities. The dielectric material, PTFE for example, provides a surface which is less likely to adhere to biological tissue during application of ablative electromagnetic energy.

The ablation portion 112 may further include a directive or isolating component (not shown) which is positioned opposite from the target tissue contact side, the antenna positioned between the isolating component and the target tissue. Such a component may be used to direct a majority of the electromagnetic energy toward the target tissue, prevent a substantial amount of electromagnetic energy from reaching adjacent tissues opposite the ablation portion 112 from the target tissue which may result in undesirable tissue damage, or both. The directive component may or may not be electrically connected to the outer conductor of transmission line 250.

The ablation portion 112 is adapted to be deliverable via the inner lumen of the guide sheath 102. More specifically, the guide sheath 102 may have a greater stiffness than ablation portion 112, thus the distal portion of guide sheath 102 generally maintains its shape and configuration adjacent the target tissue as the ablation portion 112 is advanced there through. As depicted, ablation portion 112 is preshaped to take on a specific geometric shape as the ablating portion is advanced from the exit port of the guide sheath 102, until the ablation portion 112 takes on its final shape, a circular or annular shape as shown in FIGS. 2I and 2J for example. As is discussed in greater detail below, the ablation portion 112 can be advanced only partially exiting the distal opening of sheath 102, the desired geometric shape being a curvilinear arch. Under certain circumstances, such a configuration is very advantageous. For example, since blood and heart tissue have similar water concentrations, whether ablation portion 112 is fully or only partially extended, the treatment system will remain balanced and well adapted, provided that the overall antenna length remains constant. The design of ablation portion 112 of FIGS. 2I and 2J can be used to design any of the ablating portions disclosed herein.

The ablation portion 112 may include internal structures such as Nitinol or outer sheath structures made from shape retention materials, as discussed herein, to provide for the specific geometric shape which ablation portion 112 assumes once it exits the distal opening of sheath 102. Alternatively, considering the preferable microwave based ablation system, the antenna portion itself may be composed of such shape retaining materials, the antenna structure being metallically covered or coated as necessary to enable electrical transmission of the electromagnetic energy toward the target tissue.

When fully advanced the circular orientation of the ablation portion 1102 preferably has a diameter, indicated by arrow D of FIG. 2I, ranging from about 4 mm to about 20 min. It is important to note, while discussed in terms of two separate structures, the guide sheath 102 and ablation portion 1102 can be constructed as one unit. For example, the guide sheath 102 can be bonded to the ablation portion 112, the combined unit then being advanced through a separate sheath, taking on its specifically designed geometric shape upon exiting the sheath.

The energy source (not shown) includes a microwave generator which may take any conventional form. Since biological tissue has such a high water content, when using microwave energy for tissue ablation, the optimal frequencies are generally those which are optimal for heating water. For illustration purposes only, frequencies in the range of approximately 800 MHz to 6 GHz work well. More commonly, frequencies of 915 MHz and 2.45 GHz are used. A conventional magnetron of the type commonly used in microwave ovens can be utilized as the energy source of the microwave generator. It should be appreciated, however, any other suitable microwave power source could be substituted in its place, and the disclosed concepts may be applied at other frequencies, such as 434 MHz or 5.8 GHz (ISM band) for example.

In operation, with specific reference to FIG. 2J, the guide sheath 102 is advanced toward the target tissue until the lateral outer wall of the distal portion engages the target tissue site. The ablation portion 112 is then advanced out the exit opening of the lumen and takes on its specific geometric shape, a circular geometric shape for example as shown. A first ablation procedure is then performed by supplying microwave energy to the antenna portion, which in turn radiates the energy, in part, toward the target tissue, creating a lesion therein.

After a first ablation lesion corresponding to location A in FIG. 2J is created, successive ablations can be created, as part of a desired lesion pattern, through simple movements of the guide sheath 102. For example, as depicted in FIG. 2J, the guide sheath 102 can be deflected or rotated a controlled or known amount in the direction of arrow M whereby the new position of the ablating portion 20 generally corresponds to location B of FIG. 2J. As stated above, the distal portion of sheath 102 is flexible, taking on a more acute angle with respect to the target tissue when the ablation portion 112 is moved to location B from the same access or delivery point D as shown, allowing the ablating portion 20 to maintain substantial contact with the target tissue at location B.

As depicted in FIG. 2M, as the ablation portion 112 is further moved along an ablation path in the direction or arrow M, successive continuous lesions at locations A, B and C are created. As shown, despite the fact the guide sheath 102 was simply rotated, such rotation will not necessarily result in the placement and creation of successive continuous lesions in a strict linear or straight line fashion. Rather, due to blood flow, cardiac activity or a patient's specific anatomy, the ablation portion 112 may be directed along a nonlinear path. By understanding the relationship between the angular deflection of the guide sheath 102 and the distance between the deflection point (not shown) and the ablation portion 112, the user can ensure that movement of the ablating portion in the direction M will be less than the geometric dimension of the ablating element along that line. Thus, while the ablating portion 20 may move slightly in the lateral direction along the desired ablation path and with respect to the previous ablation location, the successive ablation will still be continuous with the previous ablation.

Moreover, as depicted in FIG. 2J, certain geometric shapes, for example circular or annular shapes, in addition to providing the user more freedom of motion during an ablation procedure, also provide an extra barrier or conduction blocking ablation line, to better ensure the creation of the desired nerve conduction block. More specifically, as shown, the second ablation which is labeled B crosses or otherwise intersects the first ablation, labeled A, at least at two points, thus providing at least two barriers which act to prevent undesirable electrical signals from passing through the ablated tissue to the remaining isolated atrial tissue. It should be apparent that if the lesion B were a surface area lesion, instead of a perimeter circular lesion as depicted, then lesion B would intersect lesion A at least at two points, and arguably at numerous points. In this way a continuous lesion can be easily created by ablation systems having ablative portions adapted to assume specific geometric shapes, the geometric shapes designed to provide the user more freedom of motion and control.

While the embodiment of FIG. 2I has been depicted and described as including a guide sheath 102 having a distal curvilinear section from which the ablation portion 112 is advanced, other configurations incorporating additional steering elements are contemplated, as discussed in greater detail below. Additionally, the geometric planar shape which the ablating device forms, while shown generally normal with the longitudinal axis of the main section of the sheath 102, may be adapted to form any desirable angle therebetween from about 0° to about 90°, as generally stated above. Any of the ablation portion 112 and/or sheaths 102 disclosed herein may include deflection or steering elements which can be operated to further deflect the ablating element into various additional orientations to engage a desired target tissue surface from a specific known point with respect to the tissue. It should be apparent that such deflectable elements may be able to create area ablations while in a first configuration, and linear ablations while in another configuration, depending on the specific orientation of the ablating element. Such systems may include additional configurations corresponding to multiple area and/or linear or curvilinear ablations. The ablation devices or ablating elements disclosed herein may also comprise one or more mechanisms such as pull wires to change at least one physical dimension such as the diameter of the ablation devices or ablating elements.

The FIG. 2I or 2J embodiments may be directed or otherwise steered toward the target tissue using any suitable guiding system, such as the various steering systems currently available. Alternatively, the steering systems may utilize one or more steering wires operably attached to a handle portion allowing the user to remotely manipulate or steer a distal portion which then guides the ablating device which translates therein.

FIG. 2N shows an embodiment wherein an ablation device and a guide sheath are one operative unit. As shown, an introducing sheath is first positioned with respect to the desired target tissue, the posterior wall of the left atrium or a renal artery for example. The guide sheath 102 is then advanced through the distal end of the introducing sheath until the distal opening of the guide sheath 102 is directed toward the desired location of the target tissue, the distal opening of the guide sheath 102 defining a longitudinal axis line L3. The ablating portion 112 is then deployed into its specific geometric shape and advanced toward the target tissue until it makes contact therewith. As the ablating device 110 exists the distal opening of the guide sheath 102 it takes on its predefined annular shape. Continued advancement then acts to move the ablating portion 112 toward the target tissue.

As discussed in more detail above, the ablating portion 112 can take on its desired geometric shape through any suitable method. For example, the ablating portion 112 can use a preshaped material which allows the ablating element to take on its desired geometric shape once it exits the distal opening of the guide sheath 102. The preshaped material can be in the form of Nitinol wire, or other suitable shape retaining metal or plastic. Additionally, the dielectric portion, portion 260 of FIG. 2L for example, can be formed from a shape memory material which takes on the desired geometric shape once it exits the distal opening of the guide sheath 102. It should be apparent that such ablation systems incorporating these type of preshaped materials may include guiding sheaths, guide sheath 102 and an introducing sheath for example, which are less flexible such that they do not substantially deform while the ablating element, or ablating portion 112, passes therethrough. Alternatively, the preshaped material can be adapted to take on its shape once it reaches a specific temperature, the temperature of the surrounding blood for example. Once warmed by the blood the ablating element can then take on the desired geometric shape.

Once a first lesion is created, corresponding to the current position of ablating portion 112, the ablating portion 112 is then retracted until it no longer is in substantial contact with the target tissue. The guide sheath 102 is then either rotated along its main longitudinal axis or translated within the introducing sheath in order to define a new target tissue position. More specifically, with the ablating portion 112 retracted, the guide sheath 102 can be rotated, as shown by arrow R, to another radial position with respect to the longitudinal axis of guide sheath 102. At this point, the ablating portion 112 can then be advanced to engage the target tissue at another desired location, and another lesion can be created therein, the additional lesion being continuous with the first if desired. Alternatively, with the ablating portion 112 retracted, the guide sheath 102 can be advanced or retracted to form a new distal longitudinal axis line. The ablating portion 112 is then advanced to engage the target tissue at the subsequent location and the additional lesion is formed, continuous with the first if desired.

While the steering embodiment of FIG. 2N is generally discussed with respect to having a guide sheath 102 translating within and with respect to the introducing sheath, it should be readily apparent that the introducing sheath can be translated over and with respect to the guide sheath 102 to form or define the direction of the distal opening of guide sheath 102. Furthermore, the guide sheath 102 can be translatable over the introducing sheath as well, the introducing sheath being a more rigid structure such that it functions to conform the guide sheath 102 to the shape of the introducing sheath while translating over introducing sheath. As the distal end of sheath 102 passes over the distal end of the introducing sheath, the distal portion of guide sheath 102 takes on its preformed shape, as discussed in greater detail above.

Any of the ablating portion or antennas 112 disclosed herein may be delivered using one or more delivery methods and systems disclosed in US 2007/0219546, the entire disclosure of which is incorporated herein by reference. In one example, an antenna 112 located on a medical component 110 is introduced over a guiding element such as a guide wire. The guiding element may be anchored in the anatomy, within the vasculature for example. The position of the guiding element may be adjusted one or more times during the procedure. The position of medical component 110 relative to the guide element may be adjusted one or more times during a procedure. The guide element may be a sheath that may or may not be anchored in the anatomy. Medical component 110 may exit the sheath at one or more locations. The guide sheath itself may translate over an anchored, elongate guide element to define multiple positions of the guide sheath relative to the anchored guide element. The guide element may be a guidewire. In one embodiment, an anchoring member disclosed herein is a balloon. The balloon may be used for one or more of: anchoring one or more regions of a guide element and/or medical system 100 in the anatomy, cooling one or more regions of a device or tissue, centering one or more regions of medical system 100 inside tissue, allowing visualization of tissue, and controllably displacing tissue.

Any of the medical components 110 disclosed herein may comprise a tether to manipulate one or more regions of medical component 110. FIG. 2O shows an embodiment of a medical system 100 comprising a sheath or arm 102 that is used to introduce and position a medical component 110. In FIG. 2O, medical component 110 comprises a working element 112 (e.g. a flexible long antenna) located on a first portion of medical component 110 and a tether 114 that forms the second portion of medical component 110. In this embodiment, the first portion is integrated with the second portion. The first portion comprises a device shaft 116 that may be twisted or torqued by the user. Device shaft 116 is slidably and rotatably positioned within a lumen of arm 102. Device shaft 116 may comprise a first circuit that transmits energy to the distal region of medical component 110. In one embodiment, working element 112 is a flexible long microwave antenna and the device shaft comprises a coaxial cable that transmits microwave energy (e.g. at 915 MHz ISM band or at 2.45 GHz ISM band) to working element 112. Any of the microwave antennas disclosed herein and in U.S. Provisional Patent Application Ser. No. 61/162,241 filed on Mar. 20, 2009 titled “Methods and devices for applying energy to bodily tissues”, and in U.S. Provisional Patent Application Ser. No. 61/222,409 filed on Jul. 1, 2009 titled “Steerable medical systems for positioning medical elements in or within a body”; the entire disclosures of which are incorporated herein by reference, can be used. In FIG. 2O, medical component 110 forms a mechanically unsupported looped region beyond the distal end of arm 102. The region of medical component 110 proximal to the distal end of arm 102 is mechanically supported by arm 102. The size and/or the shape and/or the position and/or the orientation of the looped region is adjustable by one or more user directed movements. In one embodiment, this adjustment is based on an anatomical dimension. The first portion of medical component 110 comprises four pairs of mapping electrodes 118 or other elements disclosed herein. One or more mapping electrodes 118 may be located on one or more of the following locations: adjacent to an end of working element 112, spaced apart from working element 112 and over the extent of working element 112. In the embodiment shown in FIG. 2O, two pairs of mapping electrodes 118 are located adjacent to each end of working element 112. Also in the embodiment shown in FIG. 2O, two pairs of mapping electrodes 118 are located over the extent of working element 112 and surround working element 112. Mapping electrodes 118 may be used to obtain electrophysiological signals from tissue. Embodiments of medical component 110 comprising mapping electrodes 118 located in close proximity to or over working element 112, enables the user to perform both electrophysiological mapping by one or more mapping electrodes 118 and energy delivery by working element 112 without changing the position of working element 112. Thus in several method embodiments disclosed herein, working element 112 is positioned at a first location of a target tissue. Thereafter, working element 112 is used to deliver energy to the first location of the target tissue to ablate tissue and create a first lesion. One or more mapping electrodes 118 are used to map electrophysiological signals from the ablated tissue or other tissue at the first location of the target tissue while maintaining working element 112 at the first location of the target tissue. Thus, mapping electrodes 118 may be used to confirm desired ablation of tissue and/or to determine the need for additional energy delivery to tissue. When mapping electrodes 118 are present over working element 112, the user can perform both energy delivery as well as mapping without moving working element 112 and without needing an additional device for mapping. This greatly simplifies the procedure while eliminating the cost of an additional device for mapping.

Tether 114 is slidably positioned within a lumen of arm 102. In one embodiment of medical system 100 in FIG. 2O, tether 114 has a sufficient stiffness to be pushable and/or torqueable. Tether 114 may be connected to a portion of device shaft 116 or to a working element 112. Any of the tethers 114 disclosed herein may comprise one or more working elements 112 or mapping electrodes 118. Tether 114 and other steering mechanisms disclosed herein may be used to change the shape of a flexible working element 112. For example, the shape of working element 112 may be changed from one of: a linear shape, a planar non-linear shape and a non-planar non-linear shape (three dimensional) shape to one of: a linear shape, a planar non-linear shape and a non-planar non-linear shape (three dimensional) shape and vice versa. Working element 112 may be used to create ablations that are similar or dissimilar to the shape of the working element 112. For example, a working element 112 in a substantially circular configuration may be used to create a substantially circular lesion. Alternately, a working element 112 in a substantially circular configuration may be used to create a substantially round lesion. The round lesion may be created, for example, by using a higher power setting and/or higher time setting than that used for creating the substantially circular lesion. Similarly, working elements 112 in a substantially closed loop configuration may be used to create lesions that correspond to the shape of the perimeter of the substantially closed loop configuration or to the shape of the area enclosed by the substantially closed loop configuration. Thus, a linear working element 112 may be used to create one or more of: linear lesions, area ablations (e.g. by positioning linear working element 112 at multiple regions on a tissue surface or by using a bent working element 112 to create an area ablation) or volume ablations e.g. by creating deeper lesions over several regions on a target tissue.

In any of the embodiments herein, the stiffness of the region of tether 114 distal to the distal end of arm 102 may be the same as or more than or less than the stiffness of the region of shaft 116 distal to the distal end of arm 102.

In one embodiment of the device in FIG. 2O, mapping electrodes 118 and a second circuit electrically connected to mapping electrodes 118 are placed on working element 112 such that there is minimal interference from mapping electrodes 118 with a microwave field generated by working element 112. In one embodiment, the second circuit comprises electrode wires made of copper or gold or silver or of stainless steel with or without a plating of copper/gold/or/silver. The electrode wires may be located on device shaft 116 or on tether 114. In one embodiment, the electrode wires are electrically insulated from the first circuit and working element 112. Mapping electrodes 118 may be made of standard radiopaque materials such as platinum. Thus, the user may use radiographic visualization to visualize mapping electrodes 118 or other radiopaque components of the devices disclosed herein during a procedure. One or more mapping electrodes 118 may be substituted by one or more radiofrequency ablation electrodes or electrophysiological pacing electrodes that deliver controlled electrical energy for electrophysiological pacing of tissue such as cardiac tissue. Although in FIG. 2O, mapping electrodes 118 are shown only on working element 112, one or more mapping electrodes 118 may be present on one or more of: device shaft 116 and tether 114. In one method embodiment, working element 112 and one or more mapping electrodes 118 are simultaneously used for delivering microwave energy and for electrophysiological mapping of tissue respectively. In another method embodiment, one or more mapping electrodes 118 are used for electrophysiological mapping of tissue after working element 112 has delivered microwave energy. In another method embodiment, one or more mapping electrodes 118 are used for electrophysiological mapping of tissue before working element 112 has delivered microwave energy. In another method embodiment, one or more mapping electrodes 118 or other elements are used to measure the impedance of tissue.

In FIG. 2O, the distal end of tether 114 is connected to a distal end of device shaft 116. The connection is an end-to-end connection. In such a connection, there is a smooth transition between the curve of the distal end of tether 114 and the curve of the distal end of device shaft 116. In such embodiments, when one of: shaft 116 and tether 114 is advanced distally by an amount and the other of: shaft 116 and tether 114 is withdrawn proximally by a substantially similar amount, the position of working element 112 on the looped region of medical component 110 is changed without substantially changing the shape of the looped region. In such embodiments, the stiffness of the region of tether 114 distal to the distal end of arm 102 may be substantially similar to the stiffness of the region of shaft 116 distal to the distal end of arm 102.

In FIG. 2O, arm 102 comprises an opening at the distal end of arm 102 through which medical component 110 exits. In alternate embodiments, arm 102 comprises one or more openings that are located on a side wall of arm 102 rather than at the distal end of arm 102. The one or more openings may be of the same size and shape or differ in size and/or shape. The one or more openings may be arranged on arm 102 such that medical component 110 exits arm 102 at an angle to arm 102.

FIGS. 2O-2R show various method steps of accessing and/or diagnosing and/or treating one or more regions of a hollow anatomical region such as a blood vessel or a hollow cardiac or cardiovascular region. In the embodiments shown in FIGS. 2O-2R, arm 102 and medical component 110 are inserted through a blood vessel and ultimately advanced into the target location. In such methods, the position and/or the orientation of one or more regions of medical component 110 may be manipulated by one or more of: advancing or withdrawing medical component 110 relative to the distal region of arm 102, torquing shaft 116 of medical component 110, engaging one or more steering mechanisms (e.g. pull wires) on medical component 110, pulling or releasing tether 114, torquing tether 114, advancing or withdrawing arm 102, engaging one or more steering mechanisms (e.g. pull wires) on arm 102, torquing arm 102 and increasing or decreasing the size of a looped portion of medical component 110. One or more of the abovementioned manipulations may be performed simultaneously. One or more of the method steps disclosed in FIGS. 2O-2R may be performed during a medical procedure. Further, one or more of the method steps disclosed in FIGS. 2O-2R may be performed more than once during a medical procedure. Further, one or more of the method steps disclosed in FIGS. 2O-2R and elsewhere in this document and one or more of the device embodiments disclosed herein may be used to perform one or more of: a blood vessel wall ablation procedure, an electrophysiological ablation procedure, an electrophysiological mapping procedure and an electrophysiological pacing procedure. One or more of the method steps disclosed in FIGS. 2O-2R may be used to treat atrial fibrillation by ablating multiple regions in the left atrium and/or to treat atrial flutter by ablating one or more regions in the right atrium. One or more of the method steps disclosed in FIGS. 2O-2R may be used to treat hypertension and other conditions by ablating one or more portions of the renal artery wall.

The system described herein is capable or reaching every desired target region of the left atrium during ablation procedures for treating atrial fibrillation. Further, if a long microwave antenna is used as a working element 112, the resulting lesion is deep, long and transmural. Thus the present invention is capable of forming deep, long, transmural lesions in any orientation and in any location within the left or right cardiac atria for treating electrophysiological disorders such as atrial fibrillation and flutter. This dramatically reduces the time, cost and complexity of therapeutic procedures for treating electrophysiological disorders while increasing their effectiveness.

The figures in FIG. 2 series show a variety of orientations and configurations of medical system 100 that can be used for a variety of procedures disclosed herein. In FIG. 2O, medical system 100 comprising arm 102 and a looped medical component 110 is introduced into a target region. The distal portion of arm 102 has been deflected to enable contact of working element 112 with a region on wall of the target region. Thereafter, a suitable ablative energy e.g. microwave energy may be delivered to create one or more lesions. The size of the loop of medical component 110 may be reduced (as shown in FIG. 2P) or increased as desired. Further the deflection on the distal region of arm 102 may also be reduced or increased to position working element 112 at or near another target region. Thereafter, a lesion (e.g. an area ablation) may be created. A small looped medical component 110 may be steered by arm 102 and used to ablate small regions of tissue to fill in any lesion “gaps” (if any) between adjacent ablations. This position of working element(s) 112 is used to create a long “roof line” lesion on the roof of an organ. Tether 114 may be manipulated to further position one or more working elements 112 on the roof of an organ. This step may be used to enable working element 112 to conform to any irregular surface of the organ. The distal region of arm 102 may be deflected by more than 180 degrees. In one embodiment, the distal region of arm 102 is deflected by about 200-280 degrees as shown in FIG. 2Q. In FIG. 2R, medical component 110 is partially deployed from the distal end of arm 102. This arrangement may be used to create shorter lesions.

Alternate embodiments of medical systems and ablation devices 100 may be designed wherein medical component 110 does not comprise a tether 114. FIG. 2S shows an embodiment of a medical system similar to the medical system in FIG. 2O. In the embodiment in FIG. 2I, medical component 110 does not comprise a tether 114. However, any of the medical components 110 disclosed herein such as shown in FIG. 2S and FIG. 2O may comprise one or more steering mechanisms to steer or deflect a distal region of medical component 110. Examples of such steering mechanisms include, but are not limited to one or more: sufficiently stiff stylets, pull wires, hollow tubes and deflectable elongate devices. Such steering mechanisms may be designed such that they cause none or minimal interference with the energy field emitted by elements such as working element 112. In one such embodiment, one or more steering mechanism is made of a non-metallic material. For example, medical component 110 may comprise one or more internal pull wires made of a polymeric material. The embodiment shown in FIG. 2S may be used for accessing and/or diagnosing and/or treating one or more regions of the left atrium and other anatomical regions using methods similar to those described in FIGS. 2O-2R. In such methods, the position and/or the orientation of one or more regions of medical component 110 may be manipulated by one or more of: advancing or withdrawing medical component 110 relative to the distal region of arm 102, torquing a shaft of medical component 110, engaging one or more steering mechanisms (e.g. pull wires) on medical component 110, advancing or withdrawing arm 102, engaging one or more steering mechanisms (e.g. pull wires) on arm 102 and torquing or rotating arm 102. One or more of the abovementioned manipulations may be performed simultaneously. Further, one or more of the abovementioned manipulations may be performed more than once during a medical procedure. Further, one or more of the method steps disclosed in FIGS. 2O-2R and elsewhere in this document and one or more of the device embodiments disclosed herein may be used to perform one or more of: a blood vessel wall ablation procedure, an electrophysiological ablation procedure, an electrophysiological mapping procedure and an electrophysiological pacing procedure.

FIG. 2T shows a view of the medical system 100 wherein medical component 110 is fully extended from sheath 102 and forms a looped shape.

Any of the medical systems herein such as those shown in FIGS. 2O and 2S may be used to ablate tissue around the coronary sinus. In one such embodiment, medical component 110 is introduced inside the coronary sinus to ablate tissue. In another embodiment, working element 112 is positioned inside the left atrium and is used to deliver energy such that the ablation extends from the endocardial surface at least till the coronary sinus. In one such embodiment, the ablation may be trans-mural extending from the endocardial surface till the epicardial surface.

FIGS. 2U-2X show an embodiment of a method to treat a bodily region using minimally invasive techniques. The steps shown in FIGS. 2U-2X need not be performed in the sequence as shown in the figures. The performing physician may alter the treatment sequence. Also, all of the steps shown in FIGS. 2U-2X may not be preformed during a treatment. One or more steps shown in FIGS. 2U-2X may be omitted and one or more additional steps be added during the treatment by the performing physician.

The medical system shown in FIG. 2U further comprises a hollow second medical component 122 and a first medical component 110. Second medical component 122 slides over a tether or spline 114 of medical component 110. In the embodiment shown in FIG. 2U, second medical component 102 is a mapping sheath comprising a lumen through which one or more fluids such as contrast agents can be introduced into the anatomy. Second medical component 122 may be used to collect electrophysiological information about a disease and/or the outcome of a procedure. Medical component 110 comprises a long, linear active region 142. In one embodiment, active region 142 comprises a microwave antenna that is capable of ablating a long segment of tissue. The distal ends of medical component 110 and second medical component 122 are reversibly attachable in the anatomy. Medical component 110 is introduced through a first arm 102 and second medical component 122 is introduced through a second arm 102. In medical system 100 shown in FIG. 2U, the distal end of medical component 110 is reversibly attached to the distal end of second medical component 122 forming a “U-mode” configuration.

In FIG. 2U, medical system 100 is introduced through an opening into the body and is placed adjoining the target region. In the method embodiment shown in FIG. 2U, the distal end of first arm 102 lies distal to the distal end of second arm 102. Arms 102 may be manipulated by one or more of: torquing one or both arms 102, pulling and/or pushing one or both arms 102, steering the steerable distal end of one or both arms 102. The size and shape of a looped region of medical system 100 can also be further adjusted by advancing and/or withdrawing medical component 110 and/or second medical component 122. The size of a region of any medical component 110 disclosed herein (including the embodiment shown in FIG. 2U) may be expanded to fill the entirety of a lumen or a hollow bodily passage. In one embodiment, the degree of this expansion is based on an anatomical dimension (e.g. length, width, circumference, diameter, area, volume, etc.) of the bodily passage. Energy (e.g. microwave energy) is applied to ablate a portion of the target region. In one embodiment, active region 142 creates a long lesion. As with other method embodiments disclosed herein, active region 142 may be repositioned several times to create several long lesions. The lesions may be created such that there is substantial overlap or no overlap between at least two lesions. In one method embodiment, only a single long lesion is created in the region of the left atrium adjacent to the left pulmonary vein ostia. In one method embodiment, only a single long lesion is created in the wall of a renal artery.

In FIG. 2V, medical system 100 is positioned such that the distal ends of arms 102 face opposite directions. As with other method embodiments shown in FIGS. 2U-2X, arms 102 may be further manipulated by one or more of: torquing one or both arms 102, pulling and/or pushing one or both arms 102, steering the steerable distal end of one or both arms 102. The size and shape of a looped region of medical system 100 can also be further adjusted by advancing and/or withdrawing medical component 110 and/or second medical component 122. Thereafter, energy (e.g. microwave energy) is applied to ablate a portion of the target region.

In FIG. 2W, medical system 100 is positioned such that the distal ends of arms 102 are located adjacent to each other and are parallel to each other. In medical system 100 shown in FIG. 2W, the distal end of medical component 110 is detached from the distal end of second medical component 122 forming a V-mode. In one method embodiment, the position of active region 142 is changed from that in FIG. 2U to that in FIG. 2W by pulling apart the distal end of medical component 110 from the distal end of second medical component 122 and pushing second arm 102 distally and/or steering the distal end of second arm 102. As with other method embodiments shown in FIGS. 2U-2X, arms 102 may be further manipulated by one or more of: torquing one or both arms 102, pulling and/or pushing one or both arms 102, steering the steerable distal end of one or both arms 102: The size and shape of a looped region of medical system 100 can also be further adjusted by advancing and/or withdrawing medical component 110 and/or second medical component 122. In one embodiment, active region 142 creates a long lesion. Active region 142 may be repositioned several times to create several long lesions. Multiple lesions may be created using the steps shown in FIGS. 2U-2X such that there is substantial overlap between at least two lesions.

In one method embodiment, a first lesion is created that isolates the posterior region of the heart (having two left pulmonary veins and two right pulmonary veins) from the rest of the heart (having the mitral valve). It should be noted that the first lesion may comprise multiple overlapping lesions e.g. multiple long overlapping lesions. First lesion may be created using the steps shown in FIGS. 2U-2V. Further, a second lesion may be created that extends from first lesion to a region on the mitral valve annulus of the left atrium. It should be noted that second lesion may comprise multiple overlapping lesions e.g. multiple long overlapping lesions. Second lesion may be created using the step shown in FIG. 2W.

In FIG. 2X, medical system 100 is positioned such that the distal ends of arms 102 are at a distance from each other. In the configuration of medical system 100 shown in FIG. 2X, the distal end of medical component 110 is detached from the distal end of second medical component 122 forming a V-mode. As with other method embodiments, arms 102 may be further manipulated by one or more of: torquing one or both arms 102, pulling and/or pushing one or both arms 102, steering the steerable distal end of one or both arms 102. The size and shape of a looped region of medical system 100 can also be further adjusted by advancing and/or withdrawing medical component 110 and/or second medical component 122. The combination of arm 102 and second medical component 122 may be used to push active region 142 to contact the target tissue. Thereafter, energy (e.g. microwave energy) is applied to ablate a portion of the target tissue. In one embodiment, active region 142 creates a long lesion. Active, region 142 may be repositioned several times to create several long lesions.

Although FIGS. 2U-2X mostly disclose the use of devices and methods for creating long linear lesions, various devices and methods of FIGS. 2U-2X may be designed to create point lesions, long curvilinear lesions, lesions covering an area of tissue and lesions covering a volume of tissue. Two or more of such lesions may or may not overlap. When such lesions are created in the heart, one or more of the lesions disclosed herein may be transmural. One or more of the devices and methods disclosed herein may be used to perform variations of the Maze procedure to treat atrial fibrillation. Further, any suitable medical system 100 disclosed herein may be used to perform any of the method steps disclosed herein (e.g. method steps shown in FIGS. 2U-2X) and/or create any of the lesions disclosed herein.

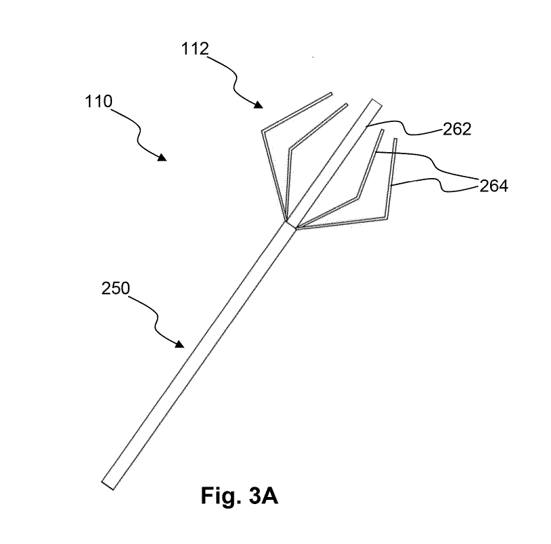

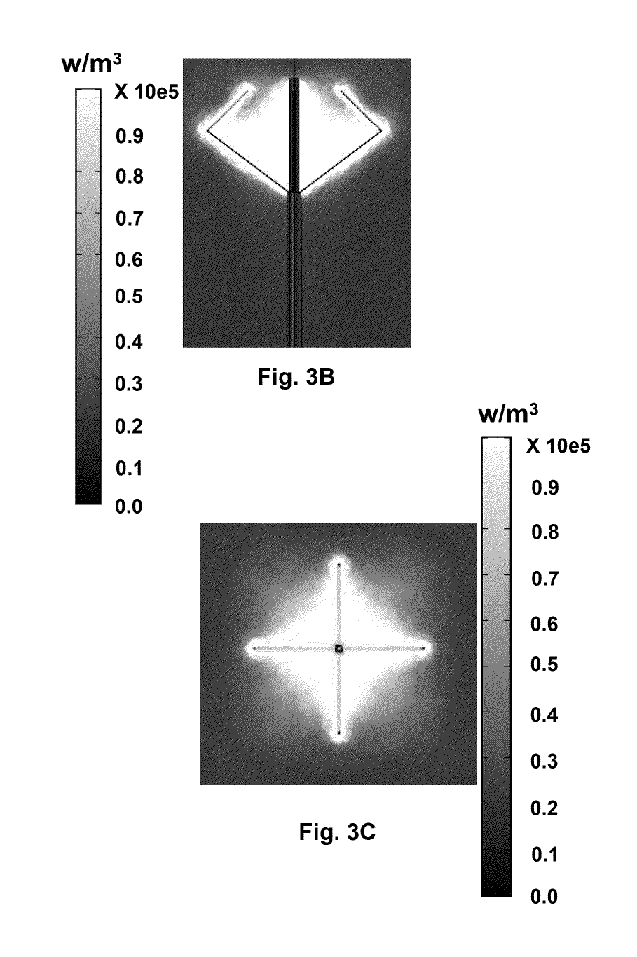

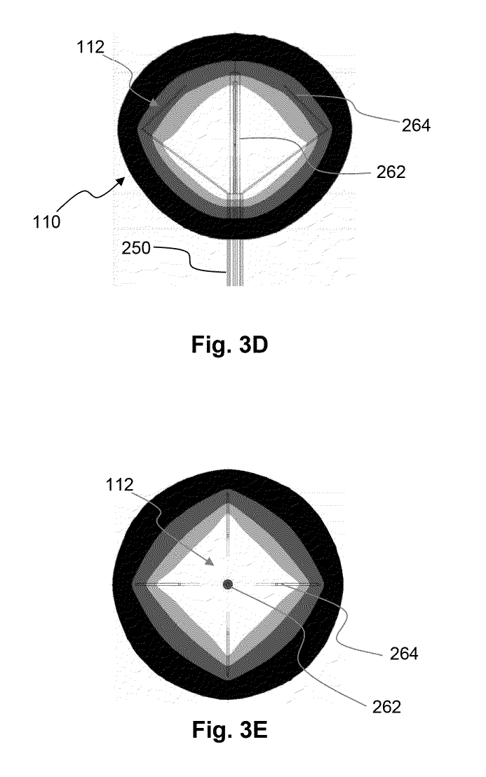

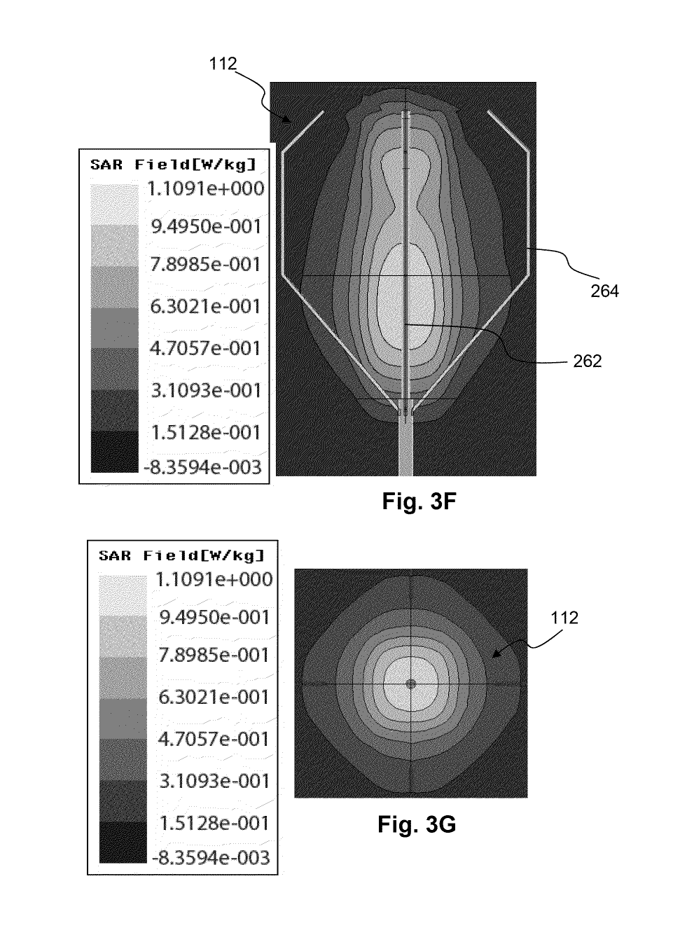

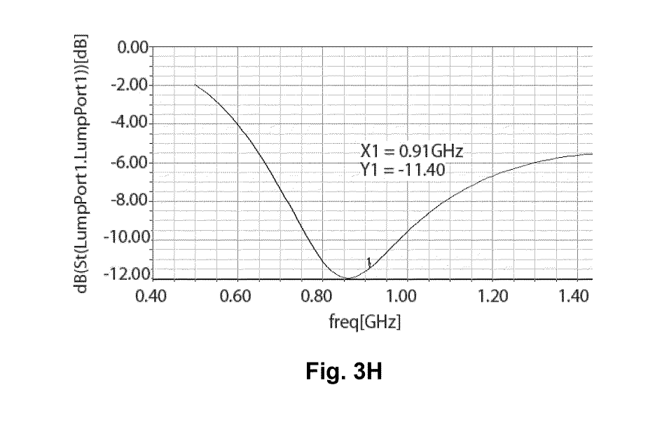

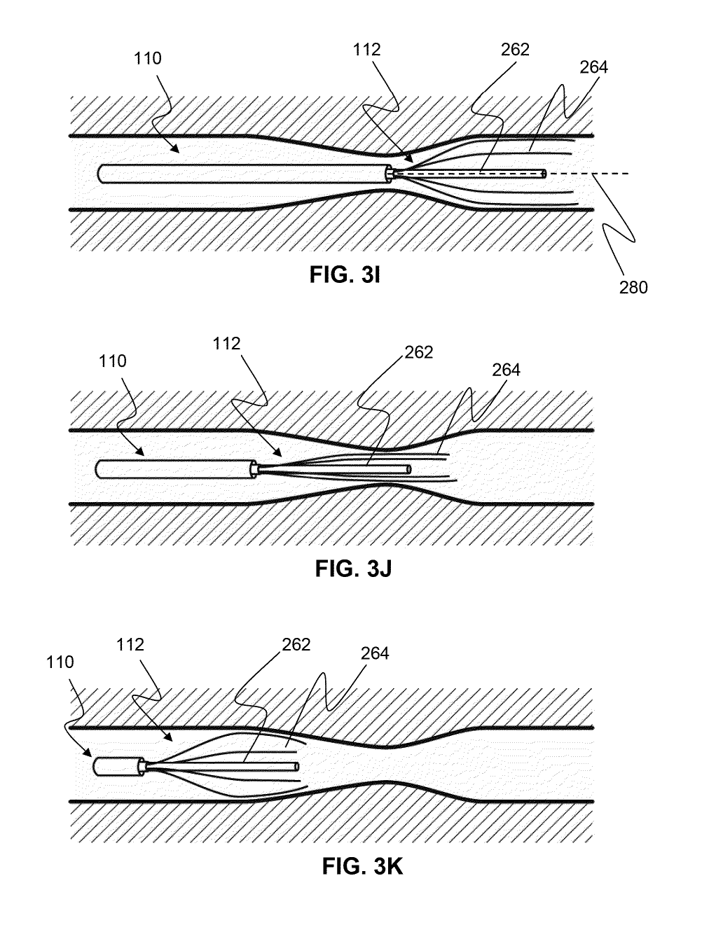

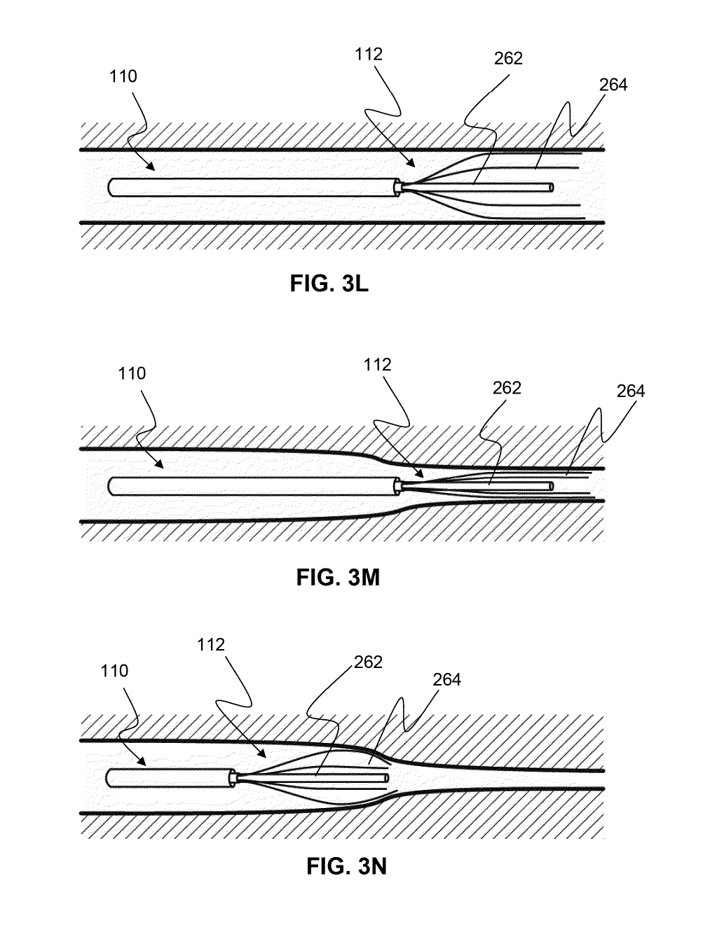

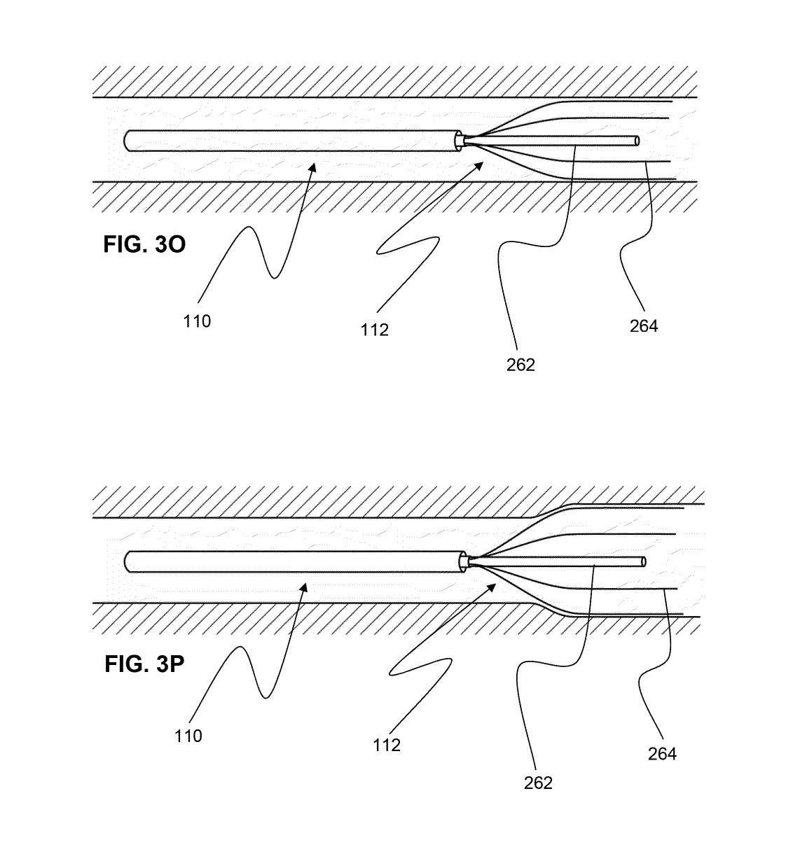

FIG. 3A shows an embodiment of an ablation device with a three dimensional antenna comprising a radiating element and multiple shaping elements adapted to ablate a volume of tissue. In FIG. 3A, ablation device 110 comprises an antenna 112 comprising a substantially linear radiating element 262. Antenna 112 further comprises a plurality of shaping elements 264. In FIG. 3A, the four shaping elements 264 are identical and are arranged symmetrically around radiating element 262. Embodiments of antenna 112 may be designed with 1-10 shaping elements 264. Shaping elements 264 may be symmetrically or non-symmetrically arranged around radiating element 262. Shaping elements 264 may or may not be identical. In FIG. 3A, each shaping element 264 is elongate and comprises a bend or an angled region. In FIG. 3A, each shaping element is electrically connected to the outer conductor of coaxial cable 250 or other transmission line. The distal end of radiating element 262 and/or shaping elements 264 may comprise a sharp or penetrating tip. In one embodiment, shaping elements 264 are a retractable claw structure that extends from an ablation device 110. In one embodiment, the design of radiating element 262 is similar to a 14 mm long monopole antenna. In FIG. 3A, shaping elements 264 shape and enhance the electromagnetic field in the volume between radiating element 262 and shaping elements 264. This creates a large, volumetric lesion between radiating element 262 and shaping elements 264. The volumetric lesion will be substantially confined to the extent of shaping elements 264 as seen from FIGS. 3B and 3C. Further, shaping elements 264 reduce the leakage current that will otherwise be induced on the outer wall of the outer conductor of coaxial cable 250 or other transmission line.

When microwave energy is delivered through a transmission line to antenna 112 in FIG. 3A, a first microwave field is emitted by radiating element 262. The first microwave field interacts with shaping elements 264. This interaction induces a leakage current on shaping elements 264. The leakage current in turn creates a second microwave field. The first microwave field and the second microwave field together combine to produce a unique shaped microwave field of antenna 112 that is clinically more useful that the unshaped microwave field generated by an antenna 112 comprising only radiating element 262. Thus the original microwave field is redistributed by the design of shaping elements 264. Shaping elements 264 alone are not capable of functioning as an antenna; rather shaping elements 264 shape or redistribute the electromagnetic or microwave field emitted by radiating element 262 to produce a shaped microwave field that is clinically more useful. Further, the combination of radiating element 262 and shaping elements 264 improves the power deposition of antenna 112 as described in other embodiments herein.

The microwave effect of shaping elements 264 can be seen by comparing FIG. 1U to FIG. 3B. In absence of shaping elements 264, antenna 112 in FIG. 3A acts as a monopole antenna similar to that shown in FIG. 1U. Thus FIG. 1U shows a first unshaped field that is not shaped by shaping elements 264. When the antenna 112 comprises shaping elements 264 as shown in FIG. 3A, the antenna generates a shaped microwave field as shown in FIG. 3B.

In an embodiment of a minimally invasive procedure, antenna 112 is inserted into the patient's body through small puncture wounds in the skin. Thereafter, antenna 112 is deployed such that the volume enclosed by the claw-like shaping elements 264 encloses the target tissue. For example, for cancer treatment, the target tissue is a tumor or a tissue with cancer cells. The degree of deployment of antenna 112 may be adjusted to suit different target tissue sizes (e.g. different tumor sizes). In one such embodiment, one or more pull wires or tethers are attached to shaping elements 264 to control the position of shaping elements 264. In another embodiment, shaping elements 264 are pre-shaped and are made of a material with shape memory properties such as Nitinol. Shaping elements 264 are retracted inside a catheter or a tubular structure in a collapsed configuration before inserting into the tissue. A low-profile catheter or a tubular structure is preferably used to reduce the trauma to healthy tissues during the insertion procedure. Once a portion of the catheter or tubular structure is inserted inside the target tissue, shaping elements 264 and radiating element 262 are deployed. Shaping elements 264 are deployed to their un-collapsed, preset shape by extending them from the catheter or tubular structure. Antenna 112 of FIG. 3A can be used for a variety of procedures including, but not limited to ablating solid tumors such as those found in cancer (e.g. liver and lung cancer) and benign tumors (e.g. uterine fibroids).

FIGS. 3B and 3C show a side view and a top view of a simulated SAR profile of an embodiment of the antenna of FIG. 3A. The SAR profile was simulated at 2.45 GHz using the COMSOL Multiphysics package to simulate an ablation in the liver. FIGS. 3B and 3C illustrate that a volumetric lesion created by antenna 112 will be substantially confined to the extent of shaping elements 264. Also, FIGS. 3B and 3C show that the microwave field volumetrically envelops entire antenna 112.

FIGS. 3D and 3E show a side view and a top view of a thermal simulation of an embodiment of the antenna of FIG. 3A. The outer most surface of the black zone is a 50° C. isosurface with a diameter or width of about 28 mm and longitudinal length of about 22 mm at steady state. Thus, antenna 112 is capable for forming a lesion with a diameter or width of about 28 mm and longitudinal length of about 22 mm. The 50° C. isosurface encloses the 60° C. isosurface (boundary between the black and dark grey zones) which in turn encloses the 70° C. isosurface (boundary between the dark grey and light grey zones) which in turn encloses the 80° C. isosurface (boundary between the light grey and white zones).

FIGS. 3F and 3G show a side view and a top view of a simulated SAR profile at 0.915 GHz of an embodiment of an antenna similar to the antenna of FIG. 3A. The SAR profile was simulated at 0.915 GHz using the Ansoft HFSS package to simulate an ablation in the liver. Radiating element 262 in FIGS. 3F and 3G is linear and has a length of about a quarter of the effective wavelength. FIGS. 3F and 3G illustrate that the volumetric lesion will be substantially confined to the extent of shaping elements 264.

The antenna 112 shown in FIGS. 3F and 3G comprises a substantially linear radiating element 262 with a plurality of shaping elements 264. The four shaping elements 264 shown in FIGS. 3F and 3G are identical and are arranged symmetrically around radiating element 262. Embodiments of antenna 112 may be designed with 1-10 shaping elements 264 arranged symmetrically or non-symmetrically arranged around radiating element 262. Shaping elements 264 may or may not be identical. In FIGS. 3F and 3G, each shaping element is elongate and comprises two bends or angled regions. Similar to the embodiment in FIG. 3A, each shaping element is electrically connected to the outer conductor of coaxial cable 250. The distal end of radiating element 262 and/or shaping elements 264 may comprise a sharp or penetrating tip. In another embodiment, the distal ends of radiating elements 262 are designed to be atraumatic. In one embodiment, shaping elements 264 are a retractable claw structure that extends from ablation device 110. In FIGS. 3F and 3G, shaping elements 264 enhance the electromagnetic field in the space between radiating element 262 and shaping elements 264. This creates a large, volumetric lesion between radiating element 262 and shaping elements 264. The volumetric lesion is substantially confined to the extent of shaping elements 264 as shown in FIGS. 3F and 3G. Further, shaping elements 264 reduce the leakage current that will otherwise be induced on the outer wall of the outer conductor of coaxial cable 250.

In FIGS. 3F and 3G, radiating element 262 comprises an elongate conductor that is about 34 mm long. The distal end of the elongate conductor is covered by a metallic tubular cap that is in conductive contact with the elongate conductor. The outer diameter of the conductive cap is about 0.8 mm and the length of the conductive cap is about 6 mm. The conductive cap is arranged such that the distance between the proximal end of the conductive cap and the distal end of the coaxial cable is about 28 mm. Entire radiating element 262 is covered with a layer of dielectric material. Each shaping element 264 comprises a proximal bend and a distal bend. The proximal bend is arranged at a longitudinal distance of about 15 mm measured along the length of the radiating element 262. The longitudinal distance between the proximal bend and the distal bend measured along the length of the radiating element 262 is about 15 mm. The longitudinal distance between the distal bend and the distal end of shaping element 264 measured along the length of the radiating element 262 is about 4 mm. Thus the total longitudinal length of each shaping element 264 measured along the length of radiating element 262 is about 34 mm. The total diameter of the structure formed by shaping elements 264 is about 30 mm. The use of antenna 112 in FIGS. 3F and 3G is similar to antenna 112 of FIG. 3A.

FIG. 3H shows the simulated return loss of an ablation device with an antenna of FIGS. 3F and 3G. The simulated return loss shows good matching (about −11.4 dB) at 0.915 GHz.

Any of the embodiments herein may use of one or more RP measurements for identification of the surrounding medium and/or automatic adjustment of energy delivery. FIGS. 3I-3K show the steps of using an antenna 112 in three different locations of different dimensions within a single target region. An antenna 112 such as an antenna disclosed in FIGS. 3A, 3F, and one or more of US 2010/0137857, US 2011/0004205 and related patent applications and a hollow target region such as a blood vessel are used only as examples to illustrate the general device and method embodiment of delivering microwave energy to multiple locations of different dimensions within a single target region. In FIG. 3I, antenna 112 is positioned at a distal location within a target region. This location has a greater dimension. Examples of dimensions include, but are not limited to: length, width, height, thickness, circumference, area, volume, diameter, etc. Antenna 112 in FIG. 3I is deployed such that one dimension (e.g. the outer diameter) of antenna 112 is greater. In FIG. 3J, antenna 112 is deployed within a target region with a smaller anatomical dimension. This leads to a change in the shape of antenna 112 by a change in the shape of one or both of: radiating element 262 and shaping element(s) 264, and antennas dielectrics 266 (if any). The shape change is defined relative to an antenna axis 280 defined as a linear axis parallel to and emerging from the distal end of the transmission line 102. For example, in FIGS. 3I and 3K, shaping elements 264 are in an axially expanded configuration or shape and in FIG. 3J, shaping elements 264 are in an axially compressed configuration or shape. This shape change leads to a change in the position of the shaping elements 264 relative to the radiating element 262 and one or more of: surrounding medium, the distal region of shielding element 256, antenna dielectrics 266 (if any), and floating conductors in the vicinity of radiating member 262 (if any). This in turn leads to a change in the electrical length of antenna 112 due to the change in the interaction of the microwave energy emitted by the radiating element 262 as described earlier. The change in the electrical length of antenna 112 and the corresponding change in the antenna impedance are detected by a change in the returned power (RP). The change in RP may be used to perform a variety of actions as disclosed elsewhere in this specification.

In the embodiments shown in FIGS. 3I-3K, RP may be measured one or more times at each location as disclosed elsewhere in this document. In one such embodiment, a RP limit (e.g. between 15%-75%) is set in the system such that energy delivery is automatically terminated if the RP limit is reached. Microwave energy may be delivered to the three sites by delivering energy to a distal site, moving antenna 112 in a discreet step from the distal site to a proximal site and thereafter delivering energy to the proximal site. In an alternate embodiment, energy may be delivered to the sites by delivering energy continuously while moving antenna 112 continuously from the distal site to the proximal site. In one embodiment of antenna 112, a wider configuration as shown in FIGS. 3I, 3K leads to a lower RP than in the narrower configuration as shown in FIG. 3J. Thus, for a same energy delivery setting, the RP limit is expected to be reached earlier at the site in FIG. 3J than at the site in FIGS. 3I, 3K. Further, for a same energy delivery setting, the power delivered is expected to be lower at the site in FIG. 3J than at the sites in FIGS. 3I, 3K. This will enable an automatic adjustment of energy delivery by delivering a larger energy dose in a larger target region and a smaller energy dose at a smaller target region. This in turn leads to a constant energy dose or power delivery at locations with larger and smaller dimensions. Thus the present invention allows for an automatically adjusted energy delivery in locations of varying dimensions. The change in RP may also be used by a user to determine the shape of a target region e.g. for determining the location of the narrowest or widest region in target region, determining the contour of the target region, etc.

In an embodiment of a procedure, antenna 112 is inserted into a material through small punctures. Thereafter, antenna 112 is deployed such that the target material is substantially enclosed or surrounded by the claw-like shaping elements 264. The degree of deployment of antenna 112 may be adjusted to suit different target material sizes. In one such embodiment, one or more pull wires or tethers are attached to shaping elements 264 to control the position of shaping elements 264. In another embodiment, shaping elements 264 are pre-shaped and are made of a material with shape memory properties such as Nitinol. Shaping elements 264 are retracted inside a sheath or a tubular structure in a collapsed configuration before inserting into the target material. Once a portion of the catheter or tubular structure is inserted inside the target material, shaping elements 264 and radiating element 262 are deployed. Shaping elements 264 are deployed to their un-collapsed, preset shape by extending them from a catheter or tubular structure or by withdrawing a catheter or tubular structure over shaping elements 264.

In one embodiment of antenna 112 of FIGS. 3I-3K, radiating element 262 comprises an elongate conductor that is about 39+/−10 mm long. The distal end of the elongate conductor may be covered by a metallic tubular cap that is in conductive contact with the elongate conductor. Entire radiating element 262 is covered with a layer of dielectric material such as silicone. Each shaping element 264 comprises a proximal bend and a distal bend. The proximal bend is arranged at a longitudinal distance of about 5 mm from the distal end of the transmission line measured along the length of the radiating element 262. The longitudinal distance between the proximal bend and the distal bend measured along the length of the radiating element 262 is about 29 mm. The longitudinal distance between the distal bend and the distal end of shaping element 264 measured along the length of the radiating element 262 is about 5 mm. Thus the total longitudinal length of each shaping element 264 measured along the length of radiating element 262 is about 39 mm. The maximum diameter of the structure formed by shaping elements 264 is about 30 mm. In this embodiment, shaping elements 264 in antenna 112 improve the matching and also reduce the return loss. Further, shaping elements 264 improve the frequency range over which important performance parameters are acceptable. Thus, a larger frequency range (bandwidth) is available over which antenna 112 delivers an acceptable performance. This in turn allows for a design of antenna 112 wherein minor deformations of antenna 112 during typical use or due to minor manufacturing variations do not significantly affect the performance of antenna 112.

FIGS. 3L-3N show the steps of a method of delivering microwave energy to a target material wherein the properties of the target material change as the microwave energy is delivered. An antenna 112 such as an antenna disclosed in FIGS. 3A, 3F, and one or more of US 2010/0137857, US 2011/0004205 and related patent applications and a hollow target are used only as examples to illustrate the general device and method embodiment of delivering microwave energy to a target region wherein the properties of the target region change as the microwave energy is delivered. In FIG. 3L, a microwave antenna 112 is positioned inside a hollow target region and is used to deliver microwave energy to the target material. In FIG. 3M, the delivered microwave energy has led to a change in one or more properties of the target region. Examples of such properties include, but are not limited to: a physical dimension, area, capacitance, concentration, density, dielectric properties, elasticity, electrical conductivity, impedance, flow rate, fluidity, friability, hardness, inductance, intrinsic impedance, length, location, loss tangent, mass, moisture content, permittivity, plasticity, resistivity, strength, stiffness/flexibility, volume. In the example shown, microwave energy delivery has led to a change in the internal dimension (e.g. the diameter) of the target region. Other examples of microwave energy based changes to tissue include, but are not limited to: changing the water content of an organ or tissue e.g. dehydrating tissue, softening of tissue, and changing the dielectric properties of tissue. In FIG. 3M, the change in material properties has led to a change in the shape of antenna 112 which in turn leads to a change in the interaction of the microwave energy emitted by the radiating element 262 relative to one or more of: radiating element 262, surrounding medium, the distal region of shielding element 256, antenna dielectrics 266 (if any), and floating conductors in the vicinity of radiating member 262 (if any). This in turn leads to a change in the electrical length of antenna 112 as described earlier. The change in the electrical length of antenna 112 and the resulting antenna impedance change are detected by a change in the returned power (RP). The change in RP may be used to perform a variety of actions as disclosed elsewhere in this specification. After the step in FIG. 3M, the antenna 112 may be moved to a different location such as shown in FIG. 3N. In another embodiment, the change in the microwave properties of the surrounding material with or without a significant change in the shape of one or more regions of antenna 112 leads to a change in the interaction of the microwave energy emitted by the antenna 112 relative to the surrounding medium. This in turn leads to a change in the electrical length of antenna 112 as described earlier. The change in the electrical length of antenna 112 and the resulting antenna impedance change are detected by a change in the returned power (RP). The change in RP may be used to perform a variety of actions as disclosed elsewhere in this specification.

In the embodiments shown in FIGS. 3L-3N, RP may be measured one or more times at each location as disclosed elsewhere in this document. In one such embodiment, a RP limit (e.g. 25+/−15%) is set in the system that automatically terminates power delivery if the RP limit is reached. Microwave energy may be delivered to the various sites by delivering energy to a first site, moving antenna 112 in a discreet step from the first site to a second site and thereafter delivering energy to the second site. In an alternate embodiment, energy may be delivered to the sites by delivering energy continuously while moving antenna 112 continuously from the first site to the second site. In one embodiment of antenna 112, a wider configuration as shown in FIG. 3L leads to a lower RP than in the narrower configuration as shown in FIG. 3M. This automatically adjusts the energy delivery as the procedure progresses till e.g. the RP limit or a set time limit is reached. Further, this allows a greater energy delivery to large target region and lower energy delivery to smaller target regions. Thus the present invention allows for an automatically adjusted energy delivery in situations wherein the properties of the target region change as the microwave energy is delivered with a consequential increase in efficiency of energy delivery by preventing wastage of energy delivery after the desired microwave effect has been achieved. One or more RP measurements may also be used by a user to determine the shape of a target region e.g. for determining the effect of the energy delivery, determining the contour of the target region, determining the end-point of a procedure, etc. These measurements may be used to automatically or manually change the microwave generator settings for performing a procedure.

The target region may be an anatomical region. In one example, the target region is a blood vessel or a duct or another hollow organ. Microwave energy delivery leads to a shrinkage of the organ due to heat induced collagen shrinkage thereby causing a narrowing or occlusion of the blood vessel or duct. In one embodiment, the system comprises a RP limit such that after a sufficient shrinkage has happened, the change in dimensions of antenna 112 lead to an increase in RP until the RP limit is reached. Thereafter, the system automatically stops further energy delivery thereby ensuring the safety of the procedure by preventing excessive energy delivery. This may be used to occlude anatomical regions such as veins (e.g. varicose veins), blood vessels feeding a benign or malignant tumor, etc. In one particular embodiment, the target region is a renal artery and the method is used to ablate one or more surrounding nerves for treating hypertension and other conditions.

FIGS. 3O and 3P show an antenna 112 similar to that in FIG. 3I in a constrained configuration and a less constrained configuration respectively. The dimensions of the target regions in FIGS. 3O and 3P and their microwave properties are the same. However, the target region in FIG. 3O is of a sufficient stiffness so that it does not get deformed when antenna 112 is deployed and the target region in FIG. 3P has a sufficient flexibility so that it gets deformed when antenna 112 is deployed. This causes antenna 112 to be deployed in a more constrained configuration in FIG. 3O as compared to the configuration in FIG. 3P. The antenna configurations in constrained and un-constrained configurations may be different due to one or more of: change in the shape of radiating element 262, change in the shape of shaping element 262, change in the position and/or shape of antenna dielectrics 266, and change in the length of antenna 112. Specifically, in the embodiments shown in FIGS. 3O and 3P, the shapes of shaping elements 264 have been changed along with the shape and/or the position of antenna dielectrics 266 (if any). This leads to a change in the position of the shaping elements 264 relative to the radiating element 262, surrounding medium, the distal region of shielding element 256, antennas dielectrics 266 (if any), and floating conductors (if any) in the vicinity of radiating member 262. This in turn leads to a change in the electrical length of antenna 112 due to the change in the interaction of the microwave energy emitted by the radiating element 262 with shaping element 264. The change in the electrical length of antenna 112 and the corresponding change in the antenna impedance are detected by a change in the returned power (RP). The change in RP may be used to perform a variety of actions as disclosed elsewhere in this specification. The embodiments in FIGS. 3O-3P further demonstrate that the RP value may be a function of both the mechanical and microwave properties of the target material.

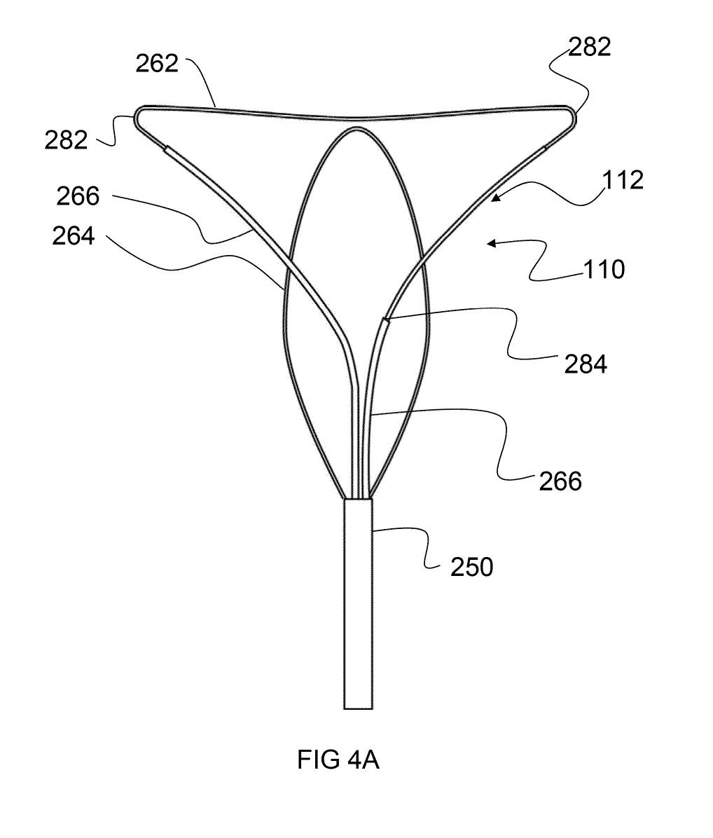

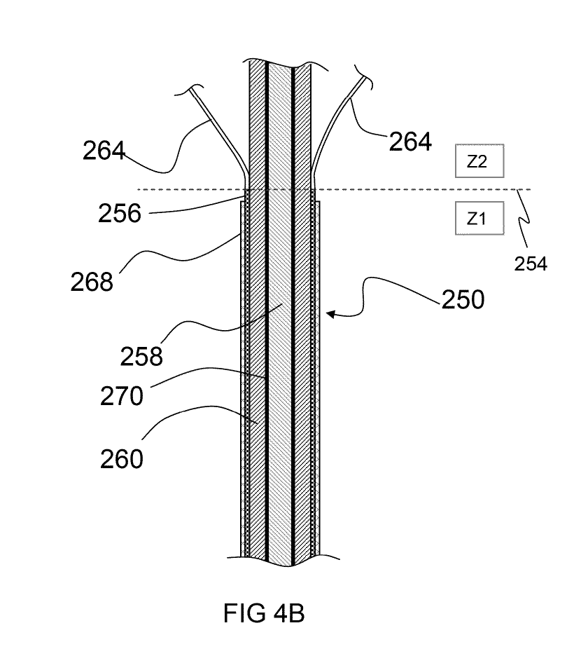

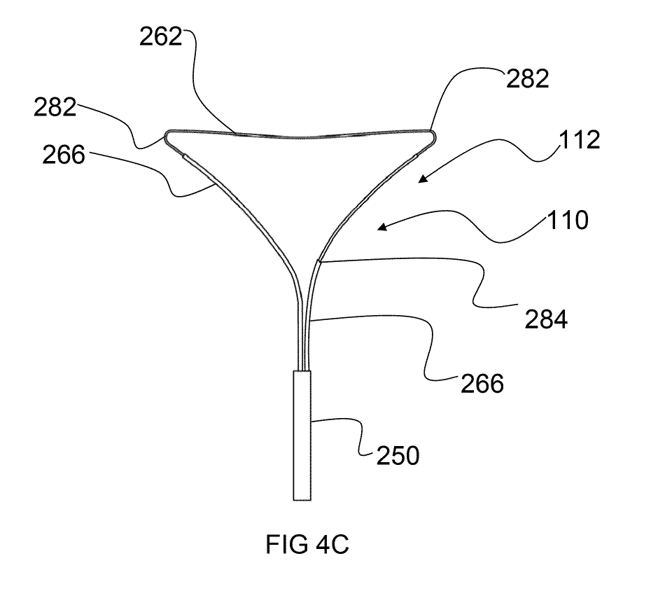

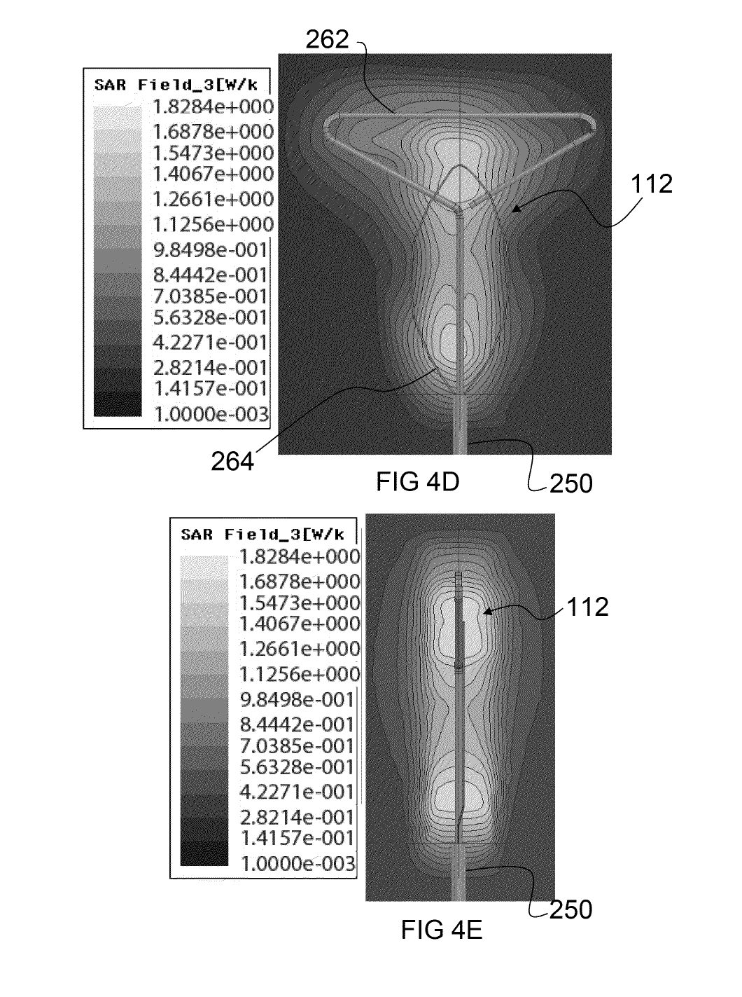

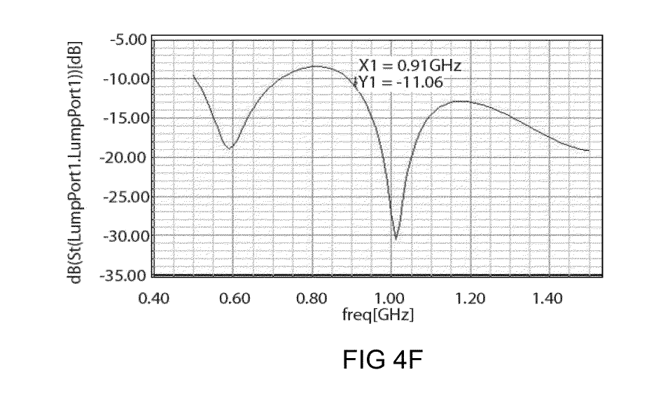

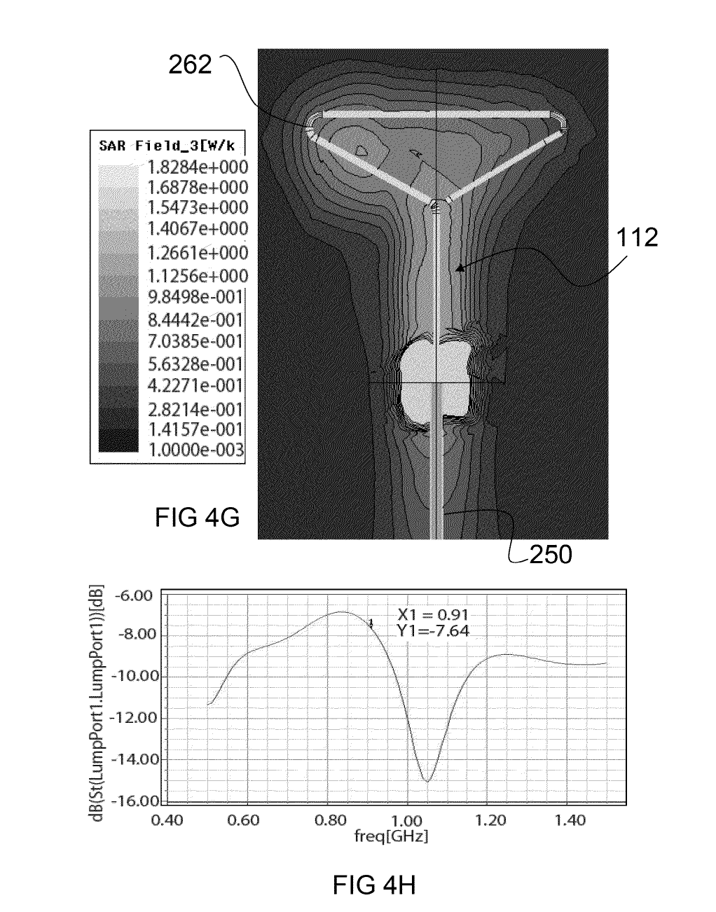

Several embodiments of planar antennas 112 are also included in the scope of the invention. Such planar antennas 112 may be used to ablate or otherwise treat planar or non-planar tissue regions. Such planar antennas 112 may comprise single or multiple splines, curves or loops in a generally planar arrangement. Planar antennas 112 may be used for ablating a surface such as the surface of organs such as liver, stomach, esophagus, a lumen of a blood vessel, etc. Planar antenna 112 may be oriented such that a flat surface of the antenna is in contact with the tissue. In another embodiment, planar antenna 112 is oriented such that an edge of the antenna is in contact with the tissue. Any of the planar antennas 112 disclosed herein may be fully or partially deployed in the anatomy. Partial deployment may occur due to one or more of: smaller size of the target organ than the size of antenna 112 that mechanically limits the deployment of antenna 112 and planned partial deployment of antenna 112 by a user. In a one embodiment, a single microwave signal is fed to an antenna 112 through a transmission line. Antenna 112 generates a microwave field. The near field portion of the microwave field generated by antenna 112 may be used for tissue ablation. For example, FIG. 4A shows a view of a planar antenna of a microwave ablation device designed for endometrial ablation. In FIG. 4A, microwave ablation device 110 comprises a transmission line (such as a coaxial cable 250) terminating in an antenna 112 at the distal end of the transmission line. In one embodiment, a single microwave signal is fed to antenna 112 through coaxial cable 250. The shape of antenna 112 is substantially triangular and has a wider distal region and a narrower proximal region. Antenna 112 generates a microwave field. The near field of the microwave field generated by antenna 112 may be used for procedure such as endometrial ablation, renal denervation, etc. In FIG. 4A, antenna 112 comprises a radiating element in the form of a bent or curved, planar outer loop 262 and a shaping element in the form of a bent or curved, planar metallic center loop 264. Outer loop 262 and center loop 264 may physically touch each other when deployed in the anatomy. In one embodiment, outer loop 262 is a continuation of the inner conductor of coaxial cable 250. Center loop 264 shapes or redistributes the microwave field radiated by outer loop 262. It should be noted that there is no direct electrical conduction between outer loop 262 and center loop 264. When microwave energy is delivered through coaxial cable 250 to antenna 112, a first microwave field is emitted by outer loop 262. The first microwave field interacts with center loop 264. This interaction induces a leakage current on center loop 264. The leakage current in turn creates a second microwave field. The first microwave field and the second microwave field together combine to produce a unique shaped microwave field of antenna 112 that is clinically more useful that the unshaped microwave field generated by an antenna 112 comprising only outer loop 262. Thus the original microwave field is redistributed by the design of center loop 264. Center loop 264 alone is not capable of functioning as an antenna; rather center loop 264 shapes or redistributes the electromagnetic or microwave field emitted by outer loop 262 to produce a shaped microwave field that is clinically more useful. Further, the combination of outer loop 262 and center loop 264 improves the power deposition of antenna 112.

It should be noted that there is no direct electrical conduction between outer loop 262 and center loop 264. Antenna 112 further comprises one or more antenna dielectrics 266 covering one or more portions of one or both of: outer loop 262 and center loop 264. In FIG. 4A, an antenna dielectric 266 covers the proximal portion of outer loop 262. Any of the antenna dielectrics 266 disclosed herein may be used to shape the microwave field and to optimize the performance of antenna 112. Any of the antenna dielectrics 266 disclosed herein may be one or more conducting polymers.

A microwave field couples to the nearest conductive path. In several embodiments of antenna 112 disclosed herein, the nearest conductive path is provided by center loop 264. Thus the microwave field couples to center loop 264 instead of coupling to the shielding element of the transmission line (e.g. the outer conductor 256 of the feeding coaxial cable 250). Therefore, minimal microwave field is coupled proximally to the shielding element of the transmission line. This in turn creates a unique, shaped or redistributed microwave field that does not significantly extend proximally to antenna 112 as shown in FIGS. 4D and 4E.

In one embodiment, outer loop 262 has no sharp corners. Sharp corners in outer loop 262 may cause the field to concentrate in the vicinity of the sharp corners. In one embodiment, the minimal radius of curvature of a corner in outer loop 262 is at least 0.5 mm. In the embodiment in FIG. 4A, the radius of curvature of corner regions 282 and 282 in outer loop 262 is about 1 mm+/−0.3 mm.

In one embodiment, antenna 112 has a shape that substantially approximates the shape of the body organ to be ablated. For example, antennas in FIGS. 4A and 4D have a roughly triangular, planar shape that approximates the roughly triangular, planar shape of the uterine cavity and is especially suited for endometrial ablation. The antennas 112 may be positioned such that the plane of the antenna is parallel to the plane of the uterine cavity. The proximal portion of the antenna 112 is directed towards the cervix and corner regions 282 and 282 of outer loop 262 are directed towards the fallopian tubes. However, as mentioned before, microwave thermal ablation does not necessarily require perfect contact with all of the target tissue. Thus antenna 112 is able to ablate all or substantially all of the endometrium. The entire endometrium can be ablated in a single ablation by antenna 112 having a single microwave antenna. Thus, repositioning of antenna 112 after an ablation is not needed. This greatly reduces the amount of physician skill needed for the procedure. Further, multiple antennas 112 are not needed in ablation device 110. A single antenna 112 positioned at a single location is able to ablate a therapeutically sufficient amount of the endometrium. This simplifies the design of ablation device 110.

In another embodiment, antenna 112 is used to deliver energy to an anatomical region that has a shape different than the shape of antenna 112. In one such embodiment, a planar antenna is inserted into a blood vessel in a compressed or low profile configuration. Thereafter, antenna 112 is deployed within the blood vessel and is used to deliver energy to one or more portions on the wall of the blood vessel.

Further, antenna 112 in the working, deployed configuration is generally flat and sufficiently flexible such that during and after introduction and deployment of antenna 112 in the anatomy, the anatomy experiences only slight forces from antenna 112. This may be achieved by designing an antenna 112 comprising one or more flexible outer loop 262, one or more flexible center loop 264 and one or more flexible antenna dielectrics 266. The plane of outer loop 262 is substantially parallel to the plane of center loop 264. Thus, the uterine walls experience only slight forces from antenna 112. This in turn reduces or eliminates the distension of the uterine wall thereby reducing the discomfort to the patient. This in turn further reduces the anesthesia requirements. Flexible antenna 112 may easily be introduced in a collapsed, undeployed configuration through a small lumen thereby eliminating or minimizing any cervical dilation. In such a collapsed, undeployed configuration, both outer loop 262 and center loop 264 are in a small profile, linearized configuration. The lack of cervical dilation dramatically reduces the discomfort to the patient consequently significantly reducing the requirement of anesthesia. This has tremendous clinical advantages since now the procedure can be performed in the physician's office under local anesthesia. In the collapsed configuration, outer loop 262 and center loop 264 may be closer to each other than in the non-collapsed configuration. This enables the introduction of antenna 112 through narrow catheters, shafts, introducers and other introducing devices. Further, this enables the introduction of antenna 112 through small natural or artificially created openings in the body.

Further, flat and flexible antenna 112 in FIG. 4A in its deployed configuration has an atraumatic distal end in which the distal region of antenna 112 is wider than the proximal portion of antenna 112. This design creates an atraumatic antenna 112 which in turn reduces the risk of perforation of the uterus or other organs. The flexible nature of antenna enables antenna 112 to take the natural shape of passage during introduction instead of distorting the passage. For example, when antenna 112 is introduced trans-cervically into the uterus, antenna 112 may acquire the shape of introduction passage comprising the vagina, cervical canal and uterine cavity instead of distorting one or more of the vagina, cervical canal and uterine cavity.

In one embodiment, the length of outer loop 262 measured along the outer loop 262 from the distal end of coaxial cable 250 or other transmission line until the distal end of outer loop 262 is an odd multiple of one quarter of the effective wavelength at one of: 433 MHz ISM band, 915 MHz ISM band, 2.45 GHz ISM band and 5.8 GHz ISM band. In one embodiment of a deployed configuration of antenna 112 as shown in FIG. 4A, the length of outer loop 262 measured along the outer loop 262 from the distal end of coaxial cable 250 until the distal end 158 of outer loop 262 is about three quarters of the effective wavelength at the 915 MHz ISM band. The effective wavelength is dependent on the medium surrounding the antenna and the design of an antenna dielectric on the outer loop 262. The design of the antenna dielectric includes features such as the type of dielectric(s) and thickness of the dielectric layer(s). The exact length of the outer loop 262 is determined after tuning the length of outer loop 262 to get good impedance matching. The length of the outer loop 262 in one embodiment is 100+/−15 mm. In one embodiment, the width of deployed outer loop 262 is 40+/−15 mm and the longitudinal length of deployed outer loop 262 measured along the axis of coaxial cable 250 is 35+/−10 mm. In the embodiment shown in FIG. 4A, distal end 158 of outer loop 262 is mechanically connected to the distal end of coaxial cable 250 by an elongate dielectric piece 160.

In one embodiment, the proximal portion of outer loop 262 is designed to be stiffer and have greater mechanical strength than the distal portion. In the embodiment shown in FIG. 4A, this may be achieved by leaving original dielectric material 260 of coaxial cable 250 on the proximal portion of outer loop 262. In an alternate embodiment, this is achieved by coating the proximal portion of outer loop 262 by a layer of antenna dielectric.

In the embodiment shown in FIG. 4A, the cross sectional shape of outer loop 262 may or may not be uniform along the entire length of outer loop 262. In this embodiment, the proximal portion of outer loop 262 is a continuation of the inner conductor of coaxial cable 250. This portion has a substantially circular cross section. A middle portion of outer loop 262 has a substantially flattened or oval or rectangular cross section. The middle portion may be oriented generally perpendicular to the distal region of coaxial cable 250 in the deployed configuration. The middle portion of outer loop 262 is mechanically designed to bend in a plane and comprise one or more bends after deployment in the anatomy. This in turn ensures that the distal most region of ablation device 110 is atraumatic and flexible enough to conform to the target tissue anatomy. This helps in the proper deployment of outer loop 262 in the uterus. In one embodiment, the middle portion of outer loop 262 is a continuation of inner conductor of coaxial cable 250 and is flattened. In one embodiment, the distal most portion of outer loop 262 is a continuation of inner conductor of coaxial cable 250 and is non-flattened such that it has a circular cross section.

One or more outer surfaces of outer loop 262 may be covered with one or more layers of antenna dielectrics 266 as shown in FIG. 4C. One or more outer surfaces of center loop 264 may be covered with one or more layers of antenna dielectrics 266. The thickness and type of antenna dielectric material along the length of outer loop 262 is engineered to optimize the microwave field shape. In one embodiment shown in FIG. 4A, every portion of outer loop 262 is covered with some antenna dielectric material such that no metallic surface of outer loop 262 is exposed to tissue. Thus, in the embodiment of FIG. 4A, outer loop 262 is able to transmit a microwave field into tissue, but unable to conduct electricity to tissue. Thus, outer loop 262 is electrically insulated from surrounding tissue. Thus, in the embodiment of FIG. 4A, there is no electrical conduction and no conductive path between outer loop 262 and center loop 264 even though outer loop 262 and center loop 264 may physically touch each other when deployed in the anatomy. Examples of dielectric materials that can be used as antenna dielectrics in one or more embodiments disclosed herein include, but are not limited to EPTFE, PTFE, FEP and other floropolymers, Silicone, Air, PEEK, polyimides, cyanoacrylates, epoxy, natural or artificial rubbers and combinations thereof. In the embodiment of FIG. 4A, the antenna dielectric 266 on the proximal portion of outer loop 262 is a continuation of the dielectric 260 of coaxial cable 250. There may be an additional layer of a stiffer antenna dielectric 266 over this later of antenna dielectric 266. In the embodiment of FIG. 4A, the dielectric on the middle portion of outer loop 262 is a silicone layer with or without impregnated air or a silicone tube enclosing a layer of air. In the embodiment of FIG. 4A, the dielectric on the distal most portion of outer loop 262 is a silicone layer with or without impregnated air or a silicone tube enclosing a layer of air or EPTFE. The thickness of an antenna dielectric on any portion of outer loop 262 may vary along the length of outer loop 262. Further, the crossection of an antenna dielectric on any portion of outer loop 262 may not be symmetric. The various configurations of the antenna dielectric are designed to achieve the desired ablation profile as well as achieve the desired impedance matching or power efficiency. In an alternate embodiment, entire outer loop 262 is covered with silicone dielectric. In one such embodiment, the layer of silicone used to coat the distal most portion of outer loop 262 may be thinner than the layer of silicone used to coat the middle portion of outer loop 262. The thinner silicone dielectric compensates for the lower field strength that normally exists at the distal most portion of a radiating element such as outer loop in FIG. 4A. Thus, the microwave field is made more uniform along the length of outer loop 262. In one device embodiment, outer loop 262 is made of a metallic material and the circumference of the metallic material of the distal region of outer loop 262 is more than the circumference of the metallic material of the middle portion of outer loop 262. This causes the silicone dielectric to stretch more at the distal portion than at the middle portion of outer loop 262. This in turn generates a thinner layer of antenna dielectric at the distal portion of outer loop 262 than at the middle portion of outer loop 262. In another embodiment, entire outer loop 262 is made from a single length of metallic wire of a uniform crossection. In this embodiment, a tubular piece of silicone dielectric of varying thickness is used to cover outer loop 262. The tubular silicone dielectric is used to cover the distal and middle portions of outer loop 262 such that the layer of silicone dielectric is thinner near the distal portion and thicker near the middle portion of outer loop 262.

In FIG. 4A, the shape of outer loop 262 is different from the shape of center loop 264. Further, in FIG. 4A, outer loop 262 and center loop 264 are both substantially planar and the plane of outer loop 262 is substantially parallel to the plane of center loop 264. Further, in FIG. 4A, both outer loop 262 and center loop 264 are bent or non-linear.

FIG. 4B shows a section of ablation device 110 of FIG. 4A through the distal end of a coaxial cable 250. Coaxial cable 250 used herein is flexible and comprises an inner conductor 258 made of Nitinol with a nickel content of 56+/−5%. The outer diameter of inner conductor 258 is 0.0172″+/−0.004″. Inner conductor 258 has a cladding or plating 270 of a highly conductive metal such as silver or gold. In one embodiment, inner conductor 258 comprises a silver cladding 270 of thickness 0.000250″+/−0.000050″. Cladding 270 in turn is surrounded by dielectric material 260. In one embodiment, dielectric material 260 is made of expanded PTFE with an outer diameter of 0.046″+/−0.005″. The dielectric material 260 in turn is surrounded by the outer conductor 256. Outer conductor 256 acts as a shielding element to the microwave signals transmitted by inner conductor 258. Further, outer conductor 256 shields the microwave signals transmitted by inner conductor 258 from external noise. In one embodiment, outer conductor 256 comprises multiple strands of gold plated copper. The multiple strands of outer conductor 256 are arranged such that the outer diameter of outer conductor 256 is 0.057″+/−0.005″. Outer conductor 256 in turn is covered by an outer jacket 268. In one embodiment, outer jacket 268 is made of PTFE with an outer diameter of 0.065″+/−0.005″. Thus, the outer diameter of coaxial cable 250 is less than about 2 mm. Similar embodiment of coaxial cable 250 may be designed that are flexible and have a diameter of less than 4 mm. Further, coaxial cable 250 is sufficiently flexible such that it conforms to a curved introduction passage comprising the vagina and the cervical canal during insertion of antenna 112 into the uterine cavity. The low profile and flexibility of the coaxial cable 250 has tremendous clinical advantages since it requires minimal or no cervical dilation during trans-cervical insertion. Coaxial cable 250 may be stiffened or strengthened if desired by adding one or more stiffening or strengthening elements such as jackets, braids or layers over coaxial cable 250. In FIG. 4B, the identity of coaxial cable 250 ends at the distal end of outer conductor 256. The outer jacket 268 ends a small distance proximal to the distal end of outer conductor 256. Inner conductor 258, cladding 270 and dielectric material 260 extend distally from the distal end of outer conductor 256 into antenna 112. Thus, the radiating element or outer loop 262 is electrically connected to inner conductor 258. Two proximal ends of center loop 264 are electrically connected to two regions on the outer conductor 256. In one embodiment, the two proximal ends of center loop 264 are electrically connected to diametrically opposite regions on the distal end of outer conductor 256. In one embodiment, the two proximal ends of center loop 264 are soldered to the distal end of outer conductor 256. In another embodiment, the two proximal ends of center loop 264 are laser welded to the distal end of outer conductor 256. The two proximal ends of center loop 264 may be connected to the distal end of outer conductor 256 in various configurations including, but not limited to lap joint and butt joint. In an alternate embodiment, at least one of the two proximal ends of center loop 264 is not connected to the distal end of outer conductor 256. For example, at least one of the two proximal ends of center loop 264 may be electrically connected to a region of outer conductor 256 that is proximal to the distal end of outer conductor 256.

In a method embodiment, when ablation device 110 is used for endometrial ablation, antenna 112 of FIG. 4A generates a substantially uniform microwave field that is more concentrated at the center of antenna 112 and is less concentrated at the corner regions 282 and towards coaxial cable 250. Thus, when used for endometrial ablation, the microwave field is more concentrated at the center of the uterus and is less concentrated towards the cornual regions and towards the cervix or the lower uterine region. Thus, the depth of ablation generated by antenna 112 is deeper in the center of the uterus and is less deep towards the cornual regions and towards the cervix. Such a profile is clinically desired for improved safety and efficacy. In one embodiment, the ablation profile is shaped to ablate a majority or entirety of the basalis layer of the uterine endometrium. The shape of the microwave field in any of the embodiments herein may be substantially similar to the shape of the uterine endometrium. In one embodiment, center loop 264 is made of a round or flat wire. Examples of flat wires that can be used to make center loop 264 are flat wires made of gold or silver plated Nitinol or stainless steel with a cross sectional profile of about 0.025″×about 0.007″. Such a loop shaped shaping element does not act as a shield for the microwave field. This non-shielding action is visible in the SAR pattern in FIG. 4D. In FIG. 4D, there is no sharp drop in the microwave field intensity past center loop 264. In the embodiment of FIG. 4A, center loop 264 is roughly oval in shape. Two proximal ends of center loop 264 are electrically attached to two circumferentially opposite regions of the outer conductor of coaxial cable 250. In the embodiment of FIG. 4A, the width of center loop 264 is 13+/−5 mm and the length of center loop 264 is 33+/−8 mm. When ablation device 110 is used for endometrial ablation, outer loop 262 and center loop 264 both contact the endometrial tissue surface.

Center loop 264 may be mechanically independent from outer loop 262 or may be mechanically attached to outer loop 262. In the embodiment shown in FIG. 4A, center loop 264 is mechanically independent from outer loop 262 and lies on one side of outer loop 262. In an alternate embodiment, a portion of center loop 264 passes through the interior of outer loop 262. In an alternate embodiment, a portion of center loop 264 is mechanically connected to outer loop 262. This may be done for example, by using an adhesive to connect a portion of center loop 264 to outer loop 262. In an alternate embodiment, one or more portions of center loop 264 are mechanically connected to one or more portions of outer loop 262 by one or more rigid or flexible dielectrics 266 that maintain the orientation between center loop 264 and outer loop 262 when antenna 112 is deployed in the anatomy.

Parts of center loop 264 may or may not be covered by one or more layers of antenna dielectric materials 266. In the embodiment of FIG. 4A, one or more or all metallic surfaces of center loop 264 are exposed to the device environment.

Portions of outer loop 262 and center loop 264 may be made from one or more of lengths of metals such as copper, Nitinol, aluminum, silver or any other conductive metals or alloys. One or more portions of outer loop 262 and center loop 264 may also be made from a metallized fabric or plastics.

FIGS. 4D and 4E show the front and side views respectively of the SAR profile generated by an antenna with a center loop similar to the antenna of FIG. 4A. In the embodiment in FIG. 4D, the distal end of outer loop 262 is mechanically and non-conductively attached to a region of outer loop 262 proximal to the distal end of outer loop 262. Thus, outer loop 262 has a substantially linear proximal region and a looped distal region. In one embodiment, the looped distal region may be substantially triangular in shape as shown in FIG. 4D. Outer diameter of antenna dielectric 266 on the proximal region of outer loop 262 may be larger than or substantially the same as the outer diameter of antenna dielectric 266 on the looped distal region of outer loop 262. Antenna dielectric 266 on the looped distal region of outer loop 262 may be a layer of silicone of varying thickness. Outer loop 262 may be made of a silver or gold clad metal such as Nitinol. Center loop 264 may be made of a silver or gold clad metal such as Nitinol. In the embodiment shown in FIGS. 4D and 4E, center loop 264 is not covered with any antenna dielectric 266. Thus the metallic surface of center loop 264 may be exposed to the surrounding. Outer loop 262 and center loop 264 may physically touch each other when deployed in the anatomy as shown in FIG. 4E. In FIG. 4D, the microwave field is shaped such that the ablation at the center of antenna 112 will be deeper than the ablation at the corners of antenna 112. This is clinically desirable for endometrial ablation. Also, FIGS. 4D and 4E show that the microwave field volumetrically envelops entire antenna 112. Also, FIGS. 4D and 4E show that the microwave field is substantially bilaterally symmetric. FIG. 4G shows the front view of the SAR profile generated by antenna 112 of FIG. 4D without center loop 264. The microwave effect of shaping element 264 in FIG. 4D can be seen by comparing FIG. 4D to FIG. 4G. FIG. 4G shows a first unshaped field that is not shaped by shaping element 264. When the antenna 112 comprises a shaping element 264 as shown in FIG. 4D, the antenna generates a shaped microwave field as shown in FIG. 4D. It should be noted that in FIGS. 4D and 4E, the shaped microwave field is more uniformly distributed over a wider area of the endometrium than in FIG. 4G. In FIG. 4G, the unshaped microwave field is more concentrated at the distal end of coaxial cable 250. A more uniformly distributed, shaped microwave field such as in FIGS. 4D and 4E is clinically desirable for endometrial ablation. Further when antenna 112 of FIG. 4D is used for endometrial ablation, the microwave field is distributed over a wider area of the endometrium that the microwave field generated by antenna 112 of FIG. 4G. This can be seen by comparing the SAR profile distal to the distal end of coaxial cable 250 in FIGS. 4D and 4E to the SAR profile distal to the distal end of coaxial cable 250 in FIG. 4G. Further, in FIG. 4G, a portion of the unshaped microwave field extends to a significant distance proximal to the distal end of coaxial cable 250. In FIGS. 4D and 4E, an insignificant portion of the microwave field extends proximally to the distal end of coaxial cable 250. Thus the microwave field profile of FIGS. 4D and 4E is advantageous over the microwave field profile of FIG. 4G since it limits collateral damage to healthy tissue. Thus the presence of center loop 264 shapes the microwave field such that the microwave field is more distributed. In absence of center loop 264, the microwave field interacts with an element of transmission line 250 such as the outer conductor of a coaxial cable. This results in a non-desirable profile of the microwave field e.g. a concentrated field around the distal end of the transmission line 250 as shown in FIG. 4G. This interaction can also cause backward heating of coaxial cable 250 that may lead to collateral damage of healthy tissue. Further, the combination of outer loop 262 and center loop 264 creates a more robust antenna 112 wherein the performance of antenna 112 is less affected by distortions during clinical use. Also, FIGS. 4D and 4E show that the microwave field volumetrically envelops entire antenna 112.

Further, the SAR profile of FIG. 4D demonstrates that the entire uterine endometrium can be ablated in a single ablation. Thus the physician can position antenna 112 at a first position and ablate substantially the entire uterine endometrium or a therapeutically sufficient amount of endometrium to treat menorrhagia. Thus the physician does not need to reposition antenna 112 after a first endometrial ablation. In one embodiment, at least a majority of endometrium is ablated. This novel aspect of the device and procedure greatly reduces the amount of time needed for the procedure and also reduces the procedure risks and physician skill requirements. In the embodiments disclosed herein, a combination of direct microwave dielectric heating and thermal conduction through tissue is used to achieve the desired therapeutic effect. The thermal conduction evens out any minor variations in the microwave field and enables the creation of a smooth, uniform ablation. Further, the SAR profile of FIGS. 4D and 4E demonstrates that antenna 112 is capable of ablating an entire volume surrounding antenna 112 not just ablating between the surfaces of outer loop 262 and center loop 264. Further, the SAR profile of FIGS. 4D and 4E demonstrates that antenna 112 is capable of ablating a tissue region without leaving any “gaps” of unablated tissue within that tissue region. Further, the SAR profile of FIGS. 4D and 4E demonstrates that the entire microwave field generated by antenna 112 is used for ablation. The entire microwave field comprises the microwave field around outer loop 262, the microwave field around center loop 264, the microwave field between outer loop 262 and center loop 264 and the field within center loop 264. Further, the SAR profile of FIGS. 4D and 4E demonstrates that the microwave field is located all around outer loop 262 and is not shielded or reflected by center loop 264. Thus center loop 264 does not act as a shield or reflector in the embodiment shown in FIGS. 4D and 4E.

Various embodiments of planar antennas 112 may be designed to generate a variety of shapes of SAR and/or the ablation profile. For example, antennas 112 may be designed to generate substantially square, triangular, pentagonal, rectangular, round or part round (e.g. half round, quarter round, etc.), spindle-shaped or oval SARs or ablation patterns.

FIG. 4F shows the simulated return loss of an ablation device with antenna 112 of FIG. 4D. The simulated return loss shows good matching (about −11 dB) at 915 MHz. FIG. 4H shows the simulated return loss of an ablation device with an antenna of FIG. 4G. The simulation shows a return loss of about −7.5 dB at 915 MHz. Thus, the presence of center loop 264 also improves the matching and increases the power efficiency. In the presence of center loop 264, microwave power is delivered more efficiently to the tissue and not wasted as heat generated within ablation device 110.

Shaping element 264 also increases the frequency range (bandwidth) over which antenna 112 delivers an acceptable performance. If the graphs in FIGS. 4F and 4H are compared, at a cutoff of −10 dB, the acceptable frequency range in the embodiment containing shaping element 264 is more than 0.52 GHz (spanning from approximately 0.88 GHz to more than 1.40 GHz). The acceptable frequency range in the comparable embodiment of FIG. 4G without shaping element 264 is only about 0.18 GHz (spanning from approximately 0.97 GHz to approximately 1.15 GHz). Thus in the first case, a larger frequency range (bandwidth) is available over which antenna 112 delivers an acceptable performance. This in turn allows for a design of antenna 112 wherein minor distortions of antenna 112 during typical clinical use or due to minor manufacturing variations do not significantly affect the performance of antenna 112.

Any of the embodiments disclosed herein may be used for ablating one or more areas within or surrounding the renal artery wall. This may be used for performing procedures such as renal denervation for treating conditions such as hypertension. FIG. 5A shows a step of one such embodiment of performing a renal denervation procedure using a linear or curved antenna 112. In such a method, arterial access is achieved and a 5 French or larger guide catheter is placed to secure the arterial access. Thereafter, a pigtail or similar catheter is advanced and an abdominal aortogram is performed to define the anatomy of aorta and renal artery origin and sizes. Thereafter, an endovascular renal denervation catheter 112 is inserted through the guide catheter and advanced into the renal artery. The denervation catheter is positioned adjacent to or in contact with the intima of the renal artery. This contact may be sensed by any of the methods listed elsewhere in this disclosure including by imaging (e.g. angiography) or by measuring a tissue or device parameter (e.g. impedence and temperature). After appropriate position is confirmed, energy is delivered by catheter 112. The energy delivered may be used to disrupt the perineurium of the renal nerve traveling in the adventia of the renal artery, thus ablating the renal nerve and making the renal nerves dysfunctional to treat hypertension and other conditions. In one embodiment, 10-20 mm region of the bilateral proximal renal artery are targeted with an ablation treatment. Using one or more embodiments disclosed herein one or more of: the proximal region, the middle region, and the distal region of one or both renal arteries may be treated. The ablation depth may range from 2 mm to 12 mm. Even though FIG. 5A shows a linear or curved antenna 112, any of the antennas 112 disclosed herein may be similarly used for performing procedures such as renal denervation. The shape of antenna 112 may be changed by manipulating an arm 102 coupled to antenna 112. Such manipulations include, but are not limited to one or more of: torquing arm 102, pulling and/or pushing arm 102, steering the steerable distal end of arm 102. The size and shape of a region of antenna 112 and/or medical component 110 can also be further adjusted by advancing and/or withdrawing medical component 110. The size of a region of antenna 112 and/or medical component 110 may be expanded based on an anatomical dimension (e.g. length, width, circumference, diameter, area, volume, etc.) of the bodily passage. In one embodiment, the expansion fills the entirety of a lumen or a hollow bodily passage with antenna 112 and/or medical component 110.

One or more device embodiments disclosed herein may be used to create one or more lesion patterns on the wall of a hollow organ (e.g. a blood vessel) or other anatomical region. FIGS. 5B-5G show various embodiments of lesion patterns that can be made on the wall of a hollow organ or other anatomical region using one or more devices and methods disclosed herein. FIG. 5B shows a series of “point” lesions (LS) created at one or more locations on the wall of a blood vessel. A point lesion is defined as a localized lesion covering less than 16 square mm in surface area. The point lesions may form a pattern. In the embodiment shown as shown in FIG. 5B, multiple point lesions form a helical lesion pattern around the wall of blood vessel (BV) or other anatomical region. The point lesions may be spaced apart to prevent strictures or undesired narrowing of the blood vessel due to collagen shrinkage. In another embodiment, multiple point lesions cover a circumferential area of a wall. To create multiple point lesions, an antenna 112 is deployed at a first region of tissue and is used to create a first point lesion. Thereafter, antenna 112 is moved to a second region of tissue using any of the methods and devices disclosed herein. Thereafter, a second lesion is created using any of the methods and devices disclosed herein. In one embodiment, one or more lesions are created that do not circumferentially envelop a wall of a vasculature to avoid causing structures due to a circumferential ablation.

FIG. 5C shows a helical lesion created on the wall of a blood vessel. To create the lesion in FIG. 5C, any of the helical antennas disclosed herein may be used. In an alternate embodiment, a non-helical antenna disclosed herein is moved along the blood vessel while being rotated to create the helical lesion.

FIG. 5D shows a circumferential lesion created on the wall of a blood vessel. The lesion in FIG. 5D may be created using an antenna 112 disclosed herein that fills the lumen of the blood vessel and is substantially adjacent to a circumferential region of the blood vessel lumen. Alternately, the lesion in FIG. 5D may be created using an antenna 112 disclosed herein that is moved or rotated to come into contact with or be adjacent to a circumferential region of the blood vessel lumen.

FIG. 5E shows multiple linear lesions created on the wall of a blood vessel. The lesions in FIG. 5D may be created using a linear antenna 112 disclosed herein. Alternately, the lesion in FIG. 5D may be created using a non-linear antenna 112 disclosed herein that is moved along a line on the wall of the blood vessel lumen. The multiple linear lesions may be parallel or non-parallel to each other. They may be of the same or different lengths and widths.

FIG. 5F shows a variety of lesions of different shapes created on the wall of a blood vessel. One or more lesions from any of the embodiments disclosed herein may be combined with one or more lesions from another embodiment disclosed herein to form a variety of lesion patterns. In the embodiment shown, a couple of point lesions are combined with a single curved lesion to form a lesion pattern. The combined lesions pattern may be decided by the user based after identifying the lesion target sites (e.g. location of nerves) using any of the methods and devices disclosed herein.

FIG. 5G shows an embodiment of an area lesion created on the wall of a blood vessel. An area lesion is defined as a localized lesion covering more than 16 square mm in surface area. The area lesion may be created by an antenna disclosed herein that ablates a region with more than 16 square mm in surface area in a single placement (i.e. without need for repositioning antenna 112). In another embodiment, an area lesion is created by an antenna disclosed herein after repositioning antenna 112 to create multiple overlapping lesions.

Any of the feedback, positioning, and energy delivery systems and methods disclosed herein may be used to create one or more lesions disclosed in FIGS. 5B-5AN and elsewhere in this specification. Any of the antennas 112 disclosed herein may be Any of the lesions disclosed herein may overlap with or may be spaced apart from an adjoining lesion. Any of the lesions disclosed herein may be created by delivering energy simultaneously while antenna 112 is being moved or repositioned. Alternately, the lesions may be created by moving antenna 112 and delivering energy only after the antenna repositioning is complete. The location of one or more lesions disclosed herein may be determined by a user based on one or more methods and devices disclosed herein.

FIG. 5H depicts a lesion patter in the left atrium. Lines LPV and RPV represent a plane defined by the openings of the left and right pulmonary veins, respectively. Line SP, shown as dashed, depicts the general approach line taken by a delivery or guide sheath 102 upon access to the left atrium from a general apical approach. If an antenna 112 is to be directed toward the LPV, the steering system comprising sheath 102 imparts a deflection of approximately 90 degrees with respect to the longitudinal axis of the delivery or guide sheath, along line SP for example, as indicated by arrow SP-LPV. Alternatively, to direct the ablating portion toward the RPV, the steering system imparts an acute angle with respect to line SP, as indicated by arrow SP-RPV. It should be apparent that deflection along one plane is all that is necessary since the deflection point directionality, for example defined by the distal opening of sheath 102, can be rotated about the line SP. This discussion generally applies to a retrograde approach as well, as more fully discussed below, since, as with the apical approach, the distal opening of a delivery or guide sheath, sheath 102 for example, is placed within the left atrium from a left ventricle approach. Also, when considering a transseptal approach, the device advanced in an antegrade manner as discussed above, where the longitudinal axis of the delivery or guide sheath is generally defined by line L1, one can observe, while the approach to the LPV is generally direct, the approach to the RPV requires deflection of approximately 140 degrees.

The ablation systems in accordance with the present invention can also be positioned via a retrograde approach. In such an approach the delivery sheath 102 would be intravenously directed into the left ventricle via the aortic arch and then deflected to eventually place the distal opening of the sheath 102 within the left atrium. The deflection can be made through any suitable means, for example via a steering catheter system using one or more pull wires to deflect the sheath 102. Additionally, the sheath can be adapted to follow a guide wire which was previously placed within the left atrium via a retrograde approach. Also, multiple sheaths can be used, sheaths similar to sheath 102 for example, employing any means of deflection discussed herein, to advance the distal opening of the delivery sheath 102 into the left atrium.

Once the distal opening of the delivery sheath 102 is positioned within the left atrium 106, the desired lesion pattern, the lesion pattern of FIG. 6E for example, can be created through movements disclosed herein.

Now turning to FIGS. 5I-5V, various exemplary flexible non-linear or planar ablating portions or antennas 112 in accordance with the present invention will be discussed. As stated above, the ablating portions are preferably formed, or otherwise adapted, to create ablation lines which have specific predetermined geometric shapes. While described as planar ablating portions, it should be apparent to one of ordinary skill that the flexible nature of the structures will allow the ablating portions to be placed substantially in contact with the target tissue surface, despite the fact that the target tissue surface is concave, as along certain locations of the left atrium endocardial wall or renal artery wall for example. The ablating portions may be used to create various area ablations, long continuous lesions, etc. within biological tissue such as cardiac tissue or a wall of a renal artery. Even though a variety of lesions have been shown as being overlapping, two or more lesions may be non-overlapping. This may be achieved by displacing an ablating portion 112 more than shown such that the subsequently created lesion does not overlap with a previously created lesion.

The ablating portions of the embodiments depicted in FIGS. 5I-5X are shown in schematic form, and can be constructed in any suitable manner, and using any modality, as discussed herein or generally known in the art. The design of ablating portion embodiments depicted in FIGS. 5I-5X can be used to design ablating portions of any of the devices disclosed herein. The ablating portions may be formed by bending or otherwise distorting a single antenna or a combination of antennas disclosed herein, such as substantially linear antennas (antenna 112 in FIG. 1A for example), planar antennas (antenna 112 in FIG. 4A for example), and flattened three dimensional antennas (antenna 112 in FIG. 3A for example). Generally, the ablating portions define two dimensional geometric shapes having an overall length and a width or other dimensional values for example, which allows for the more efficient creation of lesions in the target tissue with minimal movement and precision requirement. The overall length and width of the geometric shapes can be of any suitable dimension, the longer dimension, if any, preferably being placed substantially inline with or adjacent to the desired ablation line, as discussed below. The geometric configuration of the ablating portion allows great freedom of motion while moving along a desire ablation line. As long as the movement is less than the length of the geometric shape along the ablation line, a lesion continuous with a previous lesion will be formed. Last, for purposes of discussion only, the embodiments of FIGS. 5I-5X are shown with reference to an interface to a delivery system D. The delivery system can be any suitable system know in the art or discussed herein, such as the distal end of either the guide sheath or delivery sheath 102 described herein for example. Additionally, the longitudinal axis of the delivery system can form an angle with respect to the target tissue from about 0.degree. to about 90.degree..

Now turning specifically to FIGS. 5I and 5J, a first exemplary planar ablating portion 112 in accordance with the present invention will be discussed in greater detail. As shown, the ablating element has a general “J” shape and comprises a substantially linear section followed by a distal curved section forming the distal end of ablating portion 112. The distal curved section, while shown forming an arc of approximately 180.degree. with respect to the proximal section, can form an arc in the range of about 10 degrees to about 360 degrees, the latter being discussed in more detail below. As with the other geometric shapes defined by the embodiments of FIGS. 5I-5X, the ablating portion 112 can be rotated, or otherwise positioned, with reference to a desired ablation line AL.

With reference specifically to FIG. 5J, with the first exemplary ablating portion 112 orientated with respect to the ablation line AL corresponding to a first ablating position shown in dashed as A1, a first ablation or lesion within the target tissue can be created in a similar fashion as described above. Once created, only simple movements by the user are required to properly place the ablating element for subsequent ablations. More specifically, the user can move the ablating portion 112 along the desired lesion line AL in order to properly position the ablating portion 112 for creation of a subsequent ablation, ablation A2 for example, being continuous with the initial ablation A1, using the steering systems discussed herein for example. Similarly, additional lesions can be created through further simple user movements to create a continuous lesion, as part of a desired lesion pattern. As should be apparent from FIG. 5J, the specific geometric shape of ablating portion 112 allows for the movement of ablating portion 112 along the desired ablation line, ablation line AL for example, with less required precision, but increased efficacy with regard to the ablation procedure itself. The geometric shape increases the likelihood of creating a desired continuous lesion during beating heart procedures, as stated above, it is important to note that the actual surface ablation created can have the general geometric shape of the ablation portion itself, or can take on the overall geometric shape of the ablation portion, an exemplary overall geometric shape indicated by line AL1 of FIG. 5J, corresponding to ablation A1.

A second exemplary flexible ablating portion 112 is depicted in FIG. 5K. As shown, the element 112 comprises two generally linear sections and a curvilinear section therebetween. While shown depicting the curvilinear section positioned in the middle of the ablation portion 112, the curvilinear section can be defined anywhere along the ablating portion 112. As with the embodiment of FIG. SI, the ablating portion 112 is rotated to an orientation generally as depicted with reference to a desired ablation line AL. As the ablating element is subsequently moved to create multiple continuous ablations, at corresponding locations A1, A2 and A3 along ablation line AL of FIG. 5L for example, the overall geometric shape of the ablation portion 110 ensures that for movements less than the overall length of the ablating portion 110, there will be one intersecting point between the individual lesions.

The exemplary flexible ablating portion 112 of FIG. 5M is similar to portion 112 in FIG. 5K but provides an initial linear portion at is proximal end and includes an elongate curved portion forming its distal end. It should be apparent that the distal curvilinear section can include one or more linear sections therein. Further, the initial proximal linear section can be longer to provide for a longer individual lesion.

As discussed with reference to the embodiments of FIGS. 5I and 5K, FIG. 5N depicts an exemplary continuous lesion created by ablations A1, A2 and A3. It should be apparent from the depiction that the specific geometric structure of the ablation portion 112 allows greater freedom of motion during the ablation process.

Referring to FIGS. 5O-5T, exemplary flexible loop structures are depicted in accordance with the present invention. Along with having overall geometric lengths and widths adapted to encourage the creation of a continuous lesion along a desired ablation line, loop structures also have the ability to create at least two barriers or conduction blocks to erratic signals related to cardiac arrhythmias. With reference to FIGS. 5O and 5P, another exemplary flexible ablating portion 112 is shown comprising two linear sections and a curvilinear section there between. The curvilinear section can take on any suitable form allowing for the preferable width to length ratio to encourage the creation of continuous lesions with minimal and coarse user input.

As a desired continuous lesion is created, along ablation line AL of FIG. 5P for example, successive lesions intersect immediately proceeding lesions at two points corresponding to the general geometry of the ablating portion 112, increasing the likelihood of a successful ablation procedure. As discussed above, while the ablating portion 112 of FIG. 5O may result in the creation of a surface ablation generally corresponding to the area defined by portion 112, subsequent ablations A2 and A3, as shown, are deemed to intersect previous ablation A1 at least at 2 points.

FIG. 5Q depicts an exemplary flexible ablating portion 112 having a single curvilinear section in accordance with the present invention. While shown to be generally oval in shape, the curvilinear portion can have any suitable shape which provides the desired ratio of width to length to enable the creation of continuous lesions, in accordance with the present invention. Similarly to the embodiment of FIG. 5O, ablating portion 112 creates lesions which provide at least two intersecting points or barriers for increased efficacy, as defined herein.

The exemplary flexible ablation portion 112 of FIG. 5S is similar to the ablating element 112 of FIG. 5I. The ablation portion 112 is also similar to the exemplary embodiment of FIG. 5I, the distal curvilinear portion, however, in the FIG. 5S providing a curved portion of about 360.degree.. As with the other loop embodiments discussed herein, if desired, the movement of the ablation portion 112 between subsequent ablation procedures can be controlled to form continuous lesions having at least two barriers, such as lesions A2 and A3 as shown in FIG. 5T, for example.

As generally shown in FIG. 5T, ablating portion 150 creates a lesion including an enclosed distal curvilinear section. The curvilinear distal section is particularly advantageous since it can be used as a further guide during an ablation procedure. For example, after a first lesion is created, such as lesion A1 of FIG. 5T, the user can then direct the ablation portion 112 along the desired lesion line AL1 of FIG. 5T to a point where the curvilinear portion of ablating portion 112 encircles the starting point of lesion A1. More specifically, when the lesion A1 is created the proximal point of the ablation portion 112, visible through fluoroscopy or other techniques, can be recorded or otherwise defined through simple marking of a procedural fluoro display for example, or through computer generated means. Once recorded, the user can direct the curvilinear portion of ablation portion 112 to encircle the recorded point, the proximal point of the ablating element during the creation of ablation A1 in this example.

Additionally, as shown in FIG. 5T, as with other ablating portions disclosed herein, the ablating portion 112 can be rotated along another desired ablation line, line AL2 for example, and a further ablation A4 can be created which is continuous with the previously created ablations A1-A3. Through rotation and further placement of the ablating portion 112, a desired lesion pattern can created.

Now turning to FIGS. 5U and 5V, another exemplary flexible ablating portion 112 is shown having linear splines arranged in a “T” shape with respect to the delivery system, the delivery system located at the top and middle of the geometric “T” shape. As is discussed in more detail below, ablating elements such as ablating portion 112 being delivered from a point more central to its geometric shape are advantageous since it provides more uniform force or pressure about the ablating device to encourage contact between the ablating device and the target tissue. Moreover, such centralized systems are more natural for electrophysiologists to use, building on their past procedural development and training, since the placement involves directing the delivery point D to a desired location generally central to the ablation created. Here, the user is using the same skills in directing the point ablation system to direct the various centralized area ablation systems discussed herein.

As shown, the ablating portion 112 comprises a number of linear sections or splines. While shown having three splines with respect to delivery point D, any suitable number of splines for a given corresponding geometric shape is contemplated. The splines may also be of differing lengths and may be radially positioned in any suitable manner to achieve the desired overall geometric configuration. Moreover, the splines may be radially arranged more closely spaced with each other, each spline adapted to include a curved portion such that the spline members are parallel with respect to each other substantially over the length of each spline, as discussed with reference to FIG. 5W below.

Preferably, the spline forming the length, or longest dimension, of ablation portion 112 as depicted in FIG. 5U is slightly longer than the remaining splines. In such a configuration, as discussed with other exemplary embodiments, the longer spline is positioned substantially in line with the desired ablation line AL through simply rotational motion of the ablating portion 112, indicated by arrow R. Using methods discussed herein a continuous lesion can be formed through further simple movement and ablation of target tissue along the desired ablation line AL of FIG. 5V. In accordance with the present invention, the ablating portion 112 is adapted to be moved a fraction of its length, e.g. a fraction of the length of longer spline. The vertical displacement of the individual ablations A1-A4 more accurately portrays displacement errors due to the natural physiological motion of the beating heart and the blood flowing therein. With delivery systems of sufficient stiffness, held in place by user control or contact forces of anatomical structures, the motion of the delivery system and, thus, the ablating portion itself translating therethrough will be minimized enabling the creation of continuous lesions, as depicted in FIG. 5V for example.

Now turning to FIG. 5W, an alternative exemplary spline ablating portion is shown having three spline members which are each individual antennas 112. As depicted, two spline members include proximal curved portions with distal portions parallel to central spline member. The resultant configuration, provided ample ablative energy application, can produce an area ablation as represented by line AL1 of FIG. 5W. As discussed elsewhere herein, such geometric configurations resulting in area ablations are advantageous since they provide a higher probability of success with respect to linear ablating portions, or otherwise ablating portions having only a single spline member, the single spline member oriented along the desired ablating line.

As discussed with respect to other exemplary embodiments herein, with reduced ablative power applied the resultant ablation may be more consistent with the actual geometric configuration of spline members, the ablation for example comprising three separate linear ablation lines proximately connected and spaced therebetween. Moreover, the ablating portion 112 allows for the creation of at least one conduction block with respect to errant signals when multiple area ablations are created forming a desired continuous lesion. While each spline member is depicted as terminating equidistance from spline adjacent member, other configurations are contemplated. Alternatively, one or more of the spline members may include intermediate curved sections (not shown) which effectively widen the overall geometric shape of ablating portion 112 along its length.

As with the exemplary embodiment of FIG. 5U, the spline members include one or more ablating elements 112 thereon from which ablating energy is applied to the target tissue. While shown having three spline members, additional spline members, at any suitable individual length to define the desired geometric configuration, are also contemplated.

Turning to FIG. 5X, another exemplary embodiment depicting an ablating portion will be discussed in greater detail. As shown, the ablating portion includes two concentric curvilinear spline members 112. Such a system allows the creation of area ablations having at least two lines of conduction block with respect to a point central to the inner spline, such as a pulmonary vein ostium. Additionally, such a system, as discussed with respect to other ablating portions comprising loop sections, allows for the creation of continuous lesions having at least two lines of conduction block, preventing undesirable signals from triggering atrial fibrillation.

While the embodiments of FIGS. 5W and 5X are depicted with delivery points D located generally laterally with respect to the ablating portion geometric shape, other delivery points D are contemplated, as discussed in more detail below. For illustration purposes only, the delivery points, that is the points from which the ablating portions exit the steering or delivery systems, can be located more central to the geometric shape of the ablating portion providing a more consistent contact force between the ablating portion and the target tissue. Additionally, the ablating portions can form any suitable angle with respect to the delivery point D or the delivery point D can include a flexible joint as discussed in more detail below.

Another advantage of systems incorporating ablating portions which form ablations defining at least two lines of conduction block is such systems provide for a higher overall probability of creating a desired lesion along a lesion line as part of a desired lesion pattern. More specifically, due to the non-uniform nature of certain biological tissue surfaces, certain endocardial surfaces of cardiac tissue for example, it is often difficult to ensure that proper placement of the ablating portion with respect to the target tissue is achieved. For illustration purposes only, consider an ablation system having an ablating portion similar to ablating portion 112 of FIG. 5W, where the ablating elements require physical and direct contact with the target tissue surface. Such non-uniform tissue surfaces can impact the ability for certain splines to properly engage the tissue and create a desired lesion as part of a lesion pattern. In this case, assuming ample ablative power is applied to create overlapping lesions between each adjacent spline pair, other ablating elements on other spline members would make the desired lesion at that location upon the target tissue surface. Thus, as should be readily apparent, while the individual ablations created by individual spline member may not be continuous with respect to the individual corresponding spline member, the overall lesion created by ablating portion 112 will be continuous.

Now turning to FIGS. 5Y-5AN, additional exemplary ablating portions in accordance with the present invention are depicted. FIGS. 5Y-5AN depict ablating portion structures similar to the embodiments of corresponding FIGS. 5I-5V, however including symmetrical or non-symmetrical element structures with respect to a delivery point established along the length of the ablating portion itself. As shown with specific reference to FIGS. 5Y, 5AA and 5AC, the ablating portion may be nonsymmetrical with respect to a delivery point D. For example, the ablating portion may include a symmetrical or mirrored structure about the delivery point D as in the case of FIG. 5AA, or, alternatively, a non-symmetrical structure about the delivery point D as in the case of FIGS. 5Y and 5AC. In certain circumstances, physically establishing the delivery point more central to the ablating element itself can be more advantageous. As stated above, a more centralized delivery point provides for a more uniform force between the ablating portion and the target tissue. Additionally, it provides for easier creation of certain area ablations through the simple rotation of the ablating portion about the delivery point. Last, it provides a natural transition for electrophysiologists since the steering reference is more centralized to the ablating element, similar to point ablation devices currently in wide use. The design of ablating portion embodiments depicted in FIGS. 5Y-5AN can be used to design ablating portions of any of the devices disclosed herein.

As with FIGS. 5I-5V, the exemplary embodiments of FIGS. 5Y-5AN depict the ablating portion in outline form, however, the actual ablation created can differ from the depicted geometric shape, as discussed above. Any modality can be used, as described herein. For example, considering a radiofrequency based ablation system, the ablating element can include a number of spaced apart electrodes arranged along the length of the ablating portion. Alternatively, for further illustrative purposes, the ablating portion can comprise one or more antenna structures adapted for transmission of electromagnetic energy into biological tissue. Additionally, as with the exemplary embodiments of FIGS. 5I-5V, for each exemplary embodiment shown in FIGS. 5Y-5AN, there is a corresponding figure depicting an exemplary lesion pattern. As discussed above relative to FIGS. 5I-5V, the exemplary lesion patterns shown in dashed line are for illustration purposes only. As stated above, the actual surface ablation may differ depending on the specific arrangement of the one or more ablating elements and the ablation energy utilized and in the manner the energy is applied. The exemplary lesion patterns depict the advantages of the general structures when creating continuous lesions, as discussed herein. The ablating portions may be used to create various area ablations, long continuous lesions, etc. within biological tissue such as cardiac tissue or a wall of a renal artery. Even though a variety of lesions have been shown as being overlapping, two or more lesions may be non-overlapping. This may be achieved by displacing an ablating portion 112 more than shown such that the subsequently created lesion does not overlap with a previously created lesion.

The overall dimensions of the various exemplary embodiments of FIGS. 5Y-5AN may be similar to their counterparts in FIGS. 5I-5V, or may differ in scale or dimension. The various exemplary embodiments of FIGS. 5Y-5AN are, for illustration purposes only, depicting alternative structures adapted to the exemplary embodiments of FIGS. 5I-5V, in accordance with the present invention. For example, while FIG. 5Y is shown comprising a generally centralized delivery point D and two different ablating portion segments which extend therefrom, the exemplary embodiment of FIG. 5Y could include two identical sections arranged approximately 180.degree. radially from each other. Alternatively, the exemplary embodiment of FIG. 5Y could include a plurality of “J” type structures, as depicted in FIG. 5I, mounted about the delivery point D, for creating area ablations in accordance with the present invention.

FIGS. 5AE through 5AJ, 5AK and 5AL depict exemplary ablating portions comprising various loop structures arranged about a generally centralized delivery point D. As discussed above, along with the advantages of other ablating portions described herein adapted to define a generally planar geometric shape, the loop structures of FIGS. 5AE through 5AJ, 5AK and 5AL have the ability to create continuous lesion patterns including at least two barriers or conduction block lines, preventing undesirable signals originating from within one or more pulmonary veins passing therethrough to a substantial portion of left atrial tissue for example. Such systems increase the likelihood of a successful ablative procedure. For example, viewing the midpoint areas of the created ablation patterns, as depicted in dashed line in the corresponding figures, one can see two barriers depicted in dashed line. The embodiments of FIGS. 5AM-5AL include a number of loops or splines which allow for the creation of continuous lesions without the need of precise rotational control of the ablation portion itself. As long as the delivery point D is moved less than the overall dimension of the ablating portion along the ablation line, as continuous lesion if formed.

Additionally, as discussed above, the actual ablation characteristics created by the ablating portion are directly related to the modality used for the procedure and specific arrangement of the one or more ablating elements. For example, with specific reference to FIGS. 5AM and 5AN, for an ablation system which utilizes radiofrequency energy applied to the target tissue via several electrodes mounted along the length of each of the four linear splines, depending on how the electrodes are energized differing individual lesions will be created. If the electrodes along each spline, for illustration purposes, are energized relative to each other then a linear lesion generally corresponding to the ablating portion geometric shape will be created. However, if all the electrodes are energized, either through bipolar application from one electrode on a first spline to another electrode on a second spline or through unipolar application where all electrode currents travel to a ground plane for example, a surface ablation similar to the ablation defined by line AL1 is created. The depth of the created lesion can be established through control of the ablative power and the application period of such power.

As with any other embodiments described herein, the exemplary embodiments of FIGS. 5I-5AN can be steered or otherwise guided toward a desired target tissue location through any suitable steering system, including those disclosed herein.

Although many variations of medical system 100 are discussed and/or illustrated with a single arm 102, variations of medical system 100 may include any number of arms 102. In some variations, medical component 110 extends through arm 102 in a generally concentric or coaxial manner. However, the invention also includes arrangements of medical component 110 and arm 102 wherein medical component 110 extends parallel to arm 102, or extends along arm 102 or a portion thereof in a non-concentric manner. Arm 102 may be reversibly coupled to one or more medical component(s) 110 via a grasping structure. Examples of such grasping structures include, but are not limited to: releasable hooks, rings, or jaws. Arm 102 may be fully or partially hollow. One or more arms 102 may be arranged concentrically to manipulate medical component 110. Arm 102 may be introduced via one or more access ports and pathways.

For purposes of this specification, the term medical component 110 is intended to describe a medical device or portion thereof that is adapted to provide a visualization, diagnostic, or treatment procedure. For example, and as described below, in one variation, medical system 100 may include a medical component 110 comprising a first portion comprising a working element 112 configured to deliver energy (e.g., radiofrequency (monopolar or bipolar), resistively generated heat, microwaves, ultrasound energy, laser) to or from tissue and a second portion (such as a guidewire, rail, tether 114, etc.) that is used to manipulate the position and/or the orientation of the first portion. Alternatively, or in combination, medical component 110 may also comprise one or more sensors or information receiving elements. For example, such elements can include one or more of: electrodes (that detect and/or measure voltage, current and/or impedance), temperature sensors, pressure sensors, light measurement devices, infrared sensors, chemical sensors, radiation sensors, deformation sensors, or any other type of sensors that observe and/or measure a state or condition of tissue or the body. In one embodiment, the first portion and the second portion are parts of the same medical component 110. In one embodiment, the first portion and the second portion are integrated with each other and are non-separable without destroying medical component 110. In another embodiment, first portion and second portion are separable from each other by one or more user generated actions. In one embodiment, the first portion can advance over the second portion which functions as a rail, guide, or tether. In such a case, the first portion and second portion may be separate medical devices.

Examples of medical components 110, include, but are not limited to, therapeutic devices such as ablation devices for imparting a treatment to a target tissue, diagnostic devices such as mapping catheters for providing physiological information regarding a target tissue; positioning devices which include elements for providing additional positioning of additional medical components 110 (e.g., guidewires, rails, tethers, introducer catheters, sheaths, etc.), imaging devices, or non-imaging feedback devices (such as a Doppler catheter). Medical component 110 need not have a specific physical structure, for example an arm 102 of the inventive medical system 100 can be adapted to deploy a simple tube that administers a chemical ablating agent at a desired location or delivers an additional fluid used during, and in support of, the medical procedure (e.g. a contrast agent delivered to provide a clearer view of the anatomy in support of a procedure performed within a patient's heart). In yet additional variations, medical system 100 may comprise multiple separate medical components 110 used to provide a single diagnostic procedure or different steps of the same medical procedure. For instance, when using a radiofrequency energy modality, a first medical component 110 could include a first electrode while a second medical component 110 can include a second electrode (either the opposite or same polarity). Alternatively, one medical component 110 can include an ablation element or electrode while a second medical component 110 contains one or more mapping electrodes to assess the ablation lesion created by the first medical component 110.

Any of the arms 102 may comprise a handle portion on the proximal end of arm 102. The handle portion enables the user to control movement of the distal portion 104 of arm 102. In one embodiment, the handle portion is connected to one or more steering control mechanisms e.g. pull wires, pre-shaped tubular sheaths, inflatable balloons, slidable stylets, etc. In one embodiment, the handle mechanism allows the physician to control the maneuverable distal section 104 of arm 102 with a single hand. The movement of distal section 104 of arm 102 can occur in any three dimensional space. In a particular embodiment, arm 102 comprises two pairs of oppositely disposed pull wires. In another embodiment, arm 102 comprises three pull wires arranged 120 degrees apart around the circumference of arm 102. Accordingly, embodiments of arm 102 comprising any number of steering mechanisms or steering control mechanisms are to be considered within the scope of this invention. The proximal end of a lumen of arm 102 may open at the proximal end of hub. Hub may include one or more side ports. The one or more side ports may be used for example to introduce one or more liquids or devices into the lumen of arm 102.

In several embodiments of the present invention, a single type of steering mechanism is shown on arm 102. However, the invention is not limited to only a single type of steering mechanism on arm 102. Instead, any of the arms 102 of the present invention may employ any number and/or type of steering mechanisms or controls or actuators to produce the desired steering capability of the arm 102.

Any of the tethers 114 herein may have a circular, semicircular, oval, or a non-uniform cross section. In additional variations, a tether 114 may comprise a hollow cavity with any number of openings for delivery of one or more fluids such as gases or liquids such as contrast agents. In one such embodiment, a tether 114 comprises one or more lumens. Tether 114 may be made of a metallic or non-metallic (e.g. polymeric) material. In one embodiment, tether 114 comprises a coating (e.g. a lubricious coating) on the outer surface of tether 114. In one embodiment, tether 114 has an insignificant column strength i.e. tether 114 is floppy. In another embodiment, tether 114 is a wire-like member having a sufficient stiffness that allows for improved control of the remaining portion of medical component 110. Any of the tethers 114 disclosed herein may comprise a steering mechanism to steer or deflect a portion of tether 114. In one such embodiment, a tether 114 comprises a lumen in which a steering device is slidable positioned. The steering mechanism may be one or more of: a sufficiently stiff stylet, a pull wire, a hollow tube and a deflectable elongate device. Medical component 110 may comprise a sufficiently elastic joint between shaft 116 and tether 114. The sufficiently elastic joint allows the joint to bend at an angle of about 180 degrees when folded medical component 110 is introduced through arm 102. In one embodiment, the sufficiently elastic joint comprises a sleeve of stretchable material such as natural or artificial rubbers or polymers. Such a sleeve may enclose a support element such as a wire.

A joint between shaft 116 and tether 114 may have two configurations—a locked configuration and an unlocked configuration. The unlocked configuration loosely couples shaft 116 and tether 114 such that they can be advanced parallel to each other. After the joint exits the distal end of arm 102, the joint is converted to a locked configuration in which shaft 116 and tether 114 are more tightly coupled together. After performing a procedure, the joint is converted to the unlocked configuration to enable the user to remove shaft 116 and tether 114 from the anatomy using arm 102. In one such embodiment, the joint comprises two complementary fitting parts—one part is attached to tether 114 and the other part is attached to shaft 116. In another such embodiment, the joint is a reversibly detachable ball and socket joint. The ball portion and the socket portion of the joint may be brought closer to each other to achieve the locked configuration. The ball portion and the socket portion of the joint may be moved apart from each other to achieve the unlocked configuration.

In additional embodiments of the medical system 100, the position and/or the orientation of one or more medical components 110 disclosed herein can be locked relative to an arm 102. In one such embodiment, proximal portion 106 of arm 102 comprises a reversibly locking mechanism such as a rotating hemostasis valve. In another such embodiment, a handle 108 of an arm 102 can include such locking mechanisms.

In one embodiment, the position and/or the orientation of one or more arms 102 disclosed herein may be independently frozen in place to permit the physician to independently freeze the position and/or the orientation of each arm 102 into a desired profile or orientation. This reduces or eliminates the need by the physician to continuously hold an arm 102 in any particular profile.

Any of the devices disclosed herein may have a varying degree of flexibility along the length of the device. For example, arm 102 may comprise a relatively stiff proximal portion 106 and relatively flexible distal portion 104. This permits ease of articulation of arm 102 at the treatment site but a relatively stable proximal portion 106 from which arm 102 can be maneuvered.

In additional variations of the system, one or more arms 102 may be configured to perform medical procedures as well. For instance, an arm 102 may comprise one or more electrodes, a fluid source, a suction source, a reservoir to collect tissue, etc. In another embodiment, distal portion 104 of arm 102 may be adapted to provide suction, irrigation, contrast agents, etc. to the target site. In yet another embodiment, distal portion 104 of arm 102 comprises vision capabilities such as a fiber optic, CCD camera, or another vision source enabling visualization of one or more portions of medical system 100 and/or the anatomy during a procedure.

In embodiments wherein medical component 110 is connected to an auxiliary component (e.g., a power supply, imaging monitor, fluid source, etc.), a handle connected to medical component 110 can include the desired connector.

Any of the sheaths or arms such as arm 102 may comprise a steering mechanism. Examples of such steering mechanisms include, but are not limited to pull wires, pre-shaped tubular sheaths and stylet structures. A physician may rotate an arm 102 to cause an articulated distal section 104 to move in an arc-type motion. An arm 102 may comprise a single steering member 128 e.g. a pull wire that extends through a wall of arm 102 so that steering member 128 does not interfere with any devices located in a working lumen 130 of arm 102.

In one embodiment, steering members 128 are pull wires. In another embodiment, any of the steering members 128 are stylets slidably positioned within arm 102. The stylets have a sufficient stiffness to steer the distal region 104 of arm 102 when advanced or withdrawn relative to arm 102. One or more regions, especially one or more distal regions of the stylets may be pre-shaped. In another embodiment, the stylets can be twisted or torqued by the user to steer the distal regions of arm 102.

Any arm 102 disclosed herein may comprise a handle 108 on the proximal region of arm 102. Handle 108 enables the user to control movement of distal portion 104 of arm 102 by manipulating one or more regions or elements of handle 108. In one embodiment, handle 108 is connected to one or more steering control mechanisms e.g. pull wires, pre-shaped tubular sheaths or stylet structures. A hub may be located on the proximal end of arm 102.

Any of the arms 102 may comprise longitudinal slots or passages. Each of the longitudinal slots may enclose a pull wire 128 or other steering member. In one embodiment, pull wires 128 are made of stainless steel coated with PTFE. A pull wire enclosure may be disposed around each pull wire 128. Pull wire enclosures may be used to reduce friction during operation of the pull wires 128. Further, pull wire enclosures may be used to prevent the longitudinal slots of arm 102 from collapsing. In one embodiment, a pull wire enclosure is made of a helix or coil of stainless steel coated with PTFE. A lumen of arm 102 may be strengthened by an inner coil. Inner coil may prevent the lumen of arm 102 from collapsing. This in turn reduces the risk of forming a kink or a localized sharp bend in the lumen of arm 102, especially when distal region 104 of arm 102 is bent or deflected by a large angle such as 180 degrees or more. Such a kink or a localized sharp bend may substantially increase the friction between arm 102 and a device such as medical component 110 introduced through the lumen of arm 102. Thus preventing the formation of a kink or a sharp bend enables the smooth movement (e.g. translation, rotation) of devices within the lumen of arm 102 even when distal region 104 of arm 102 is bent or deflected by a large angle. Inner coil may also provide sufficient strength to arm 102 such that arm 102 may be pushed or pulled or torqued when introduced inside the anatomy. In one embodiment, inner coil is made of stainless steel.

Any of the planar embodiments disclosured herein may be capable of being deflected from a planar configuration as shown to a non-planar configuration. This may be achieved by engaging a first steering mechanism (e.g. a pull wire, a stylet, an inflatable balloon, etc.). Any of the medical systems 100 disclosed herein may comprise arms 102 that can be converted from a substantially linear configuration to a configuration in which distal portion 104 comprises more than one curve. In one embodiment, a medical system 100 disclosed herein comprises an arm 102 that can be converted from a substantially linear configuration to a configuration in which distal portion 104 is spiral or helical. In the embodiments wherein arm 102 comprises multiple steering mechanisms, one or more steering mechanisms may be different. For example, an arm 102 may be designed using a pull wire and a slidable stylet. In one such embodiment, the distal most portion of the stylet is always located proximal to the distal most portion of the pull wire. In the embodiments wherein arm 102 comprises multiple steering mechanisms, one or more steering mechanisms may be similar or the same. For example, an arm 102 may be designed using two pull wires oriented at varying circumferential positions around arm 102. Any of the pull wires disclosed herein may be oriented substantially parallel to the length of arm 102 spanned by the pull wire. Any of the pull wires may be oriented in a non-parallel orientation relative to the length of arm 102 spanned by the pull wires. Any of the medical systems 100 disclosed herein may comprise one or more bendable arms 102 wherein the radius of bending may be varied by the user.

Any of arms 102 disclosed herein may be pre-shaped. Various combinations of embodiments of arm 102 disclosed herein can be used to create medical systems 100. For example, medical system 100 may be designed with a preshaped arm 102 comprising three steering members 128.

In any of the embodiments of medical systems 100 comprising multiple arms 102 disclosed herein, two or more of the multiple arms 102 may be similar. In one embodiment, all of the multiple arms 102 of a medical system 100 comprising multiple arms 102 are similar in design. In another embodiment, none of the multiple arms 102 of a medical system 100 comprising multiple arms 102 are similar in design.

Various combinations of embodiments of arms 102 disclosed herein can be used to create medical systems 100. For example, a medical system 100 may be designed with a first preshaped arm 102 and a second steerable arm 102 comprising three steering members 128. In another embodiment, a medical system 100 may be designed with a first steerable arm 102 comprising a single steering member 128 and a second steerable arm 102 comprising two steering members 128. In another embodiment, a medical system 100 may be designed with a first preshaped arm 102 steered by a single pre-shaped stylet and a second steerable arm 102 comprising two steering members 128.

Arms 102 can be coupled via first and/or second medical components 110 when inserted into the body. Alternatively, medical system 100 is configured so that the medical component(s) 110 can be advanced in and out of (or distal and proximal to) first and second arms 102. This construction allows a physician to remove the medical component 110 from the first arm 102 or second arm 102 to ease insertion of the respective arms 102 into the patient's body. For example, the arms can be inserted into separate entry point or each arm can be separately maneuvered to the target site where the arms are ultimately coupled. In other words, medical system 100 can be inserted into a patient without the arms 102 being coupled by the medical component 110. Once the arms 102 are positioned at or near the target site, medical component 110 can be advanced from first steerable arm 102 to second arm 102. In some variations, medical component 110 can be affixed to one arm 102 so that it engages that arm 102 without extending through the entire length of that arm 102. Alternatively, medical component 110 can be advanced from first arm 102, through second arm 102 until medical component 110 extends out of handle 108 of second arm 102.

The stiffness of a region of first medical component 110 adjacent to the distal region 104 of one of arms 102 may be substantially different from the stiffness of a region of medical component 110 adjacent to the distal region 104 of the other of arms 102. In this manner, designs of medical system 100 are possible where a physical property (e.g. stiffness, torquability, outer diameter, cross-sectional shape, radio-opacity, pushability/column strength, presence of one or more lumens, presence of one or more coatings, presence of one or more markers, lubricity, outer surface properties, porosity, storability, conductivity, presence of one or more metallic elements, dielectric constant, presence of one or more exit ports, color, elasticity, presence of one or more functional elements, etc.) of the region of first medical component 110 adjacent to the distal region 104 of one of arms 102 is substantially the same as or different from the physical property of the region of medical component 110 adjacent to the distal region 104 of the other of arms 102. Embodiments of medical system 100 comprising long linear active regions 142 disclosed herein can be designed to enable a user to place long linear active region 142 in any orientation and any position on the surface of a bodily tissue. Such a medical system 100 is advantageous to treat the interior of hollow organs or anatomical cavities in a minimally invasive manner. Arms 102 are introduceable into such hollow organs or anatomical cavities (e.g. lumens) through small sized natural or artificial openings. Thereafter, active region 142 can be placed on one or more locations in the hollow organs or anatomical cavities (e.g. lumens) to treat one or more locations in the hollow organs or anatomical cavities (e.g. lumens).

Any of the arms 102 disclosed herein may comprise one or more plastically deformable distal portions 104. The plastically deformable portions are designed such that they deform elastically while being introduced into the target tissue but can be deformed plastically by the user on application of a sufficient force. Thus, in this embodiment, distal portions 104 are user shapeable.

In any of the embodiments herein, medical component 110 may be adapted to deliver two or more treatment modalities. The two or more modalities may be selected from the group consisting of: radiofrequency energy, microwave energy, a cryogenic zone, laser, ultrasound energy, IR energy, visible light, thermal energy, X-rays and other ionizing radiation and chemicals.

Any of the components disclosed herein that comprise electrodes may be designed such that the electrodes project out of the outer surface of the component to ensure better proximity to target tissue. Alternately, the components may be designed such that the electrodes do not project out of the outer surface of the components.

Two or more radiofrequency electrodes 112 disclosed herein may be coupled together or energized by a single source. Radiofrequency electrodes 112 may be designed for multi-phase radio-frequency ablation by having multiple radiofrequency electrodes 112 capable of producing multiple current paths in the ablation zone. Radiofrequency electrodes 112 are fed by one or more conducting wires extending through a length of medical component 110. In the embodiments disclosed herein, radiofrequency electrodes 112 are fed by anywhere from one to thirty two conducting wires. Several variations are possible wherein more than one radiofrequency electrodes 112 are fed by a single conducting wire. In the embodiments herein comprising multiple radiofrequency electrodes 112, adjoining radiofrequency electrodes 112 may be spaced apart such that the treatment zones formed by adjacent radiofrequency electrodes 112 overlap. For example, the ablation zone formed by two adjoining radiofrequency ablation electrodes 112 may overlap due to heat spread in target tissue to form a single large ablation zone. The multiple radiofrequency ablation electrodes 112 may operate in a monopolar or bipolar configuration. In another embodiment, multiple radiofrequency ablation electrodes 112 are capable of operating in two modes: a monopolar mode or a bipolar mode. A user may select the mode (monopolar or bipolar) depending on his need. For example, the user may select the monopolar mode for creating deep lesions and the bipolar mode for creating surface lesions. In the case of bipolar radiofrequency electrodes 112, adjacent radiofrequency electrodes 112 may be electrically coupled. In an alternate embodiment of bipolar radiofrequency electrodes 112, non-adjacent radiofrequency electrodes 112 may be electrically coupled. Multiple radiofrequency ablation electrodes 112 may be energized simultaneously. In another embodiment, multiple radiofrequency ablation electrodes 112 are energized sequentially. In another embodiment, only a few selected multiple radiofrequency ablation electrodes 112 are energized at a time. When one or more of the multiple radiofrequency ablation electrodes 112 are not energized, the non-energized electrodes may have a high impedance. This may be used, for example, to reduce interference from the non-energized radiofrequency ablation electrodes 112. In one embodiment, medical component 110 comprises multiple radiofrequency electrodes 112 coupled to a reference electrode that is located in the pericardial region. In an alternate embodiment, medical component 110 comprises a single reference electrode coupled to an array of multiple radiofrequency electrodes 112 located in the pericardial or other regions adjacent to a target organ. The voltages used to energize the multiple radiofrequency ablation electrodes 112 may be same or different. The frequencies of radiofrequency energy used to energize the multiple radiofrequency ablation electrodes 112 may be same or different. In one embodiment, the action of one or more multiple radiofrequency ablation electrodes 112 is controlled by controlling the voltage applied to the one or more multiple radiofrequency ablation electrodes 112. The one or more radiofrequency electrodes 112 disclosed herein may be energized by a variety of controlled waveforms. In one embodiment, the waveforms used to energize one or more radiofrequency electrodes 112 are changeable. The one or more electrodes 112 and associated waveforms may be designed to provide one of the following actions: cutting, coagulating, desiccating and fulgurating tissue. The one or more electrodes 112 and associated waveforms may also be designed to provide multiple actions described above, for example a combination of cutting and coagulating. One or more electrodes 112 may be designed such that they transmit and/or receive energy uniformly over their surface. Alternately, one or more electrodes 112 may be designed such that they transmit and/or receive energy non-uniformly over their surface. For example, a portion of one or more electrodes 112 may be insulated such that one or more electrodes 112 transmit and/or receive energy only along particular regions.

Medical component 110 may comprise a single, long radiofrequency electrode. Radiofrequency electrode 112 may have a length ranging from 5 to 80 mm. Medical component 110 may further comprise additional long radiofrequency electrodes 112. The long radiofrequency electrodes may be arranged in a spiral configuration.

Any of the functional elements 112 disclosed herein may be heated using resistive heating by an electrical current. The resistively heated electrode may then be used for thermal treatment of tissue.

One or more radiofrequency electrodes 112 may be slidably positioned on medical component 110. This allows the user to change the position of one or more radiofrequency electrodes 112 relative to medical component 110. This can be used, for example, to access multiple target regions of the anatomy without changing the position of medical component 110. Thus multiple target regions of the anatomy may be treated without changing the position of medical component 110. In one embodiment, a slidable shaft comprising a single radiofrequency electrode 112 or an array or radiofrequency electrodes 112 is located within a portion of medical component 110.

Any of the device embodiments herein may comprise a cooling mechanism. Examples of such cooling mechanisms include, but are not limited to: active cooling and passive cooling mechanisms Any of the embodiments herein may be cooled by irrigation with a coolant such as saline. The coolant may be released into the exterior of the embodiment or may be circulated within the interior of the embodiment. In one method embodiment, a medical component 110 is positioned such that it is spaced away from a target tissue (e.g. a wall of a vascular region) to allow blood to flow between the target tissue and the medical component 110. In this embodiment, the flowing blood acts as a cooling mechanism. In one embodiment, a medical component 110 is used along with a cooling mechanism to cool the surface of tissue. Deeper ablations are created while protecting the surface of the tissue using the cooling mechanism. In one embodiment, a fluid channel within a device embodiment acts as a cooling mechanism. In one embodiment, an inflatable balloon around or within one or more regions of medical system 100 acts as a cooling mechanism.

Any of the embodiments disclosed herein may comprise a balloon that encloses one or more portions of a device. The balloon may be inflated and/or deflated to provide one or more of the following: displace or otherwise reposition tissue relative to a device region, displace blood away from a device region, improve energy deposition, align an antenna at the center of a bodily passage, deliver drugs or other chemicals, improve microwave matching, provide a cooling mechanism, enable visualization of surrounding anatomy, and deploying an antenna disclosed herein.

Medical systems 100 disclosed herein may be used to treat a variety of internal organs including, but not limited to stomach, gall bladder, colon, rectum, urinary bladder, uterus and other regions of the female reproductive tract, regions of the male reproductive tract, esophagus, heart, lungs, liver, spleen, small intestine, and other pleural, visceral or peritoneal organs. The procedures may be performed on the surface of such organs, inside lumens or cavities of such organs or within an interior portion of such organs.

While some variations of medical system 100 comprises a tether 114 that has little column strength (essentially used to pull the device), alternate variations of tether 114 include a wire-like member having a sufficient stiffness that allows for improved control of an end of a medical component 110 that is coupled to tether 114.

In any of the embodiments disclosed herein, a looped medical component 110 and/or looped second medical component 122 may be deployed in an anatomical region such that the looped medical component 110 and/or looped second medical component 122 substantially span the entire anatomical region. In one method embodiment, this is done by deploying arms 102 in the anatomical region with a looped medical component 110 and/or looped second medical component 122 connecting the distal regions of arms 102. Thereafter, looped medical component 110 and/or looped second medical component 122 are expanded in the anatomical region till looped medical component 110 and/or looped second medical component 122 substantially span the entire anatomical region.

Although a significant amount of the disclosure discloses various embodiments of medical systems 100 used for treating regions of the heart, any device embodiment described herein may also be used to treat other anatomical regions. The devices and methods disclosed herein are especially suited for minimally invasive surgery inside hollow organs, openings or lumens. Examples of such lumens include, but are not limited to lumens in the vasculature, lumen of the gastrointestinal tract, lumens in the mouth or other oropharyngeal regions and lumens in the respiratory system. Medical system 100 may be collapsed for introduction through narrow passages or openings. Embodiments of medical system 100 may be used to ablate tissue inside or around the coronary sinus. In a particular embodiment medical system 100 is used in the V-mode to ablate tissue inside a coronary sinus.

A medical system 100 disclosed herein may be introduced non-invasively into the lower esophagus through the nose or mouth to destroy Barrett's tissue. The position and/or the orientation of one or more regions of medical system 100 is tracked using an endoscope. The position and/or the orientation of one or more regions of medical system 100 can also be tracked using fluoroscopy, ultrasound or other imaging modalities disclosed herein. Medical component 110 comprising an active region 142 is manipulated such that active region 142 is adjacent to or in contact with the target tissue. Thereafter, active region 142 is used to treat tissue. The treatment may be tracked using endoscope and/or fluoroscopy or other imaging modalities disclosed herein. After a first region of target tissue is treated, active region 142 may be moved to a second region. The repositioning can be made by any one of the methods disclosed herein. More specifically, active region 142 may be rotated circumferentially along the esophagus or moved along the length of the esophagus. Medical component 110 may be deployed in a helical or spiral configuration inside the esophagus. In one embodiment, medical component 110 has a pre-shaped region that enables medical component to achieve such a helical or spiral configuration. In another embodiment, medical component 110 is twisted by twisting or torquing one of arms 102 such that medical component 110 achieves such a helical or spiral configuration.

Any of the medical components disclosed herein may be pre-shaped so that they deploy in the anatomy in their pre-shaped configuration. Examples of such pre-shaped configurations include, but are not limited to: spiral, helical, looped, planar curved, and three dimensional.

A medical system 100 disclosed herein may be introduced non-invasively into the uterus through the vagina and the cervix for endometrial ablation. The position and/or the orientation of one or more regions of medical system 100 may be tracked using an external ultrasound device. The position and/or the orientation of one or more regions of medical system 100 can also be tracked using fluoroscopy, hysteroscopy, trans-vaginal ultrasound or any other imaging modalities disclosed herein. Arms 102 may have one or more distance markers along the length of arms 102 to indicate the depth of insertion of arms 102 into the uterine cavity. Active region 142 may be positioned inside the uterus and oriented perpendicular to the path of insertion of arms 102. After a first region of target tissue is destroyed, active region 142 may be moved to a second region and a second region of the endometrium may be ablated. More specifically, active region 142 may be pulled proximally towards the cervix.

A medical system 100 disclosed herein may be introduced non-invasively into the lower esophageal region enclosed by the lower esophageal sphincter through the nose or mouth for treating GERD (Gastroesophageal reflux disease). Arms 102 may have one or more distance markers along the length of arms 102 to indicate the depth of insertion of arms 102 into the esophagus. In this embodiment, active region 142 is used to deliver energy that causes shrinkage of tissue. Active region 142 may be rotated within the esophagus if needed.

Any of the medical systems 100 disclosed herein may comprise arm coupling elements to mechanically couple two or more arms 102 disclosed herein.

A looped medical system 100 comprising arms 102 and a elongate medical component 110 with or without a second medical component 122 may be introduced into the target anatomical site as a pre-assembled system. Such a pre-assembled medical system 100 may be collapsible and flexible for introduction through narrow lumens and cavities. In an alternate method embodiment, two arms 102 are introduced in the body. Thereafter, the distal regions of arms 102 are connected by one or more elongate devices to create a looped medical system 100 in the body. In another method embodiment, a looped access is created between 2 openings in the body using a looped auxiliary device. Thereafter, the looped auxiliary device is used to create looped medical system 100 in the body such that arms 102 exit the body through the 2 openings. In one method embodiment of using a looped auxiliary device to create looped medical system 100 in the body, a looped access is created between 2 openings in the body using the looped auxiliary device. Thereafter, arms 102 are introduced through the 2 openings over the looped auxiliary device. Thereafter, arms 102 are navigated to the target anatomy. Thereafter, looped auxiliary device is exchanged for medical component 110 or a combination of medical component 110 and second medical component 122.

Any of the embodiments of medical system 100 described herein that comprise more than one arm 102 (e.g. medical system 100 in FIG. 2U) may be modified such that a single arm 102 is used to introduce medical component 110 and other components of medical system 100 disclosed herein. In one such embodiment, a medical component 110 is disposed within an arm 102 such that two elongate regions of medical component 110 are enclosed within arm 102 and a looped portion of medical component 110 extends beyond the distal end of arm 102. In another such embodiment, a medical component 110 is disposed within an arm 102 such that one of the elongate regions of medical component 110 is enclosed within arm 102 and the other of the elongate regions of medical component 110 is located outside arm 102. The two elongate regions of a looped medical component 110 may be present in a single lumen or in two separate lumens or compartments of arm 102.

Further, any of the embodiments of medical system 100 described herein that comprise a single arm 102 (e.g. medical system 100 in FIG. 2O) may be modified such that more than one arm 102 is used to introduce medical component 110 and other components of medical system 100 disclosed herein. In one such embodiment, an additional arm 102 mechanically cooperating with a first arm 102 is used to manipulate the position and/or the orientation of one or more regions of a medical component 110.

Embodiments of medical system 100 herein may provide many degrees of freedom to position medical component 110 and/or working element 112 in any number of positions or profiles from a linear shape to simple two dimensional curves to complex three dimensional profiles that conform to a targeted region of tissue.

Any of devices disclosed herein such as medical component 110 or arm 102 may comprise one or more of any of the steering mechanisms disclosed herein. Such steering mechanisms may be designed such that they do not interfere with the working of medical component 110. For example, in embodiments wherein medical component 110 emits microwave energy, medical component 110 may comprise one or more pull wires or other steering mechanisms made of non-metallic materials such as plastic or polymer materials.

Any of the devices (e.g. medical component 110 or an arm 102) or components (e.g. a tether 114) disclosed herein may have additional features that enable the user to obtain information about one or more of: position and/or orientation of one or more components of medical system 100, contact and/or proximity of one or more components of medical system 100 to anatomical regions, images of anatomical regions, real-time information of tissue properties (e.g. temperature, electrical activity, viability, echogenicity, etc.), a physiological parameter of the patient's body, presence of one or more devices, feedback of the success or efficacy of a procedure and risky situations arising from too much force or pressure on the anatomy. In one embodiment, a component of medical system 100 comprises a force sensor to measure forces transmitted along the shaft or the one or more components of medical system 100. This may be used to determine tissue contact by the one or more components. In another embodiment, a component of medical system 100 comprises a temperature sensing modality such as a thermocouple. In another embodiment, a component of medical system 100 comprises a pressure sensor. In another embodiment, a component of medical system 100 comprises an impedance sensor. Such an impedance sensor can be used to determine the success of efficacy of an ablation procedure. In another embodiment, a component of medical system 100 comprises an embedded intra-cardiac echography probe. In another embodiment, a component of medical system 100 comprises an embedded imaging probe capable of imaging in the non-visible electromagnetic spectrum. In an embodiment, a device or component disclosed herein comprises one or more electrodes. In one such embodiment, a device or component disclosed herein comprises multiple electrodes wherein at least two of the multiple electrodes differ in their design. In another such embodiment, a device or component disclosed herein comprises multiple electrodes wherein at least three of the multiple electrodes differ in their design.

Any of the method embodiments disclosed herein may comprise the step of using a feedback before, during or after the step of energy delivery. The feedback may be obtained about the state of one or both of bodily tissue and a component of medical system 100. Examples of such feedback include, but are not limited to: measuring voltage, current, or impedance; temperature measurement; pressure measurement; measurement of one or more parameters of returned power; light measurement; measuring a chemical change; measuring a change in the imaging characteristics; measuring a change in the mechanical properties of tissue; and measuring deformation. The feedback may be collected by a component that is a part of the medical system 100 or an additional device that is separate from medical system 100. The feedback may be used to adjust one or more procedure parameters including, but not limited to energy dose, duration of energy delivery, and power level of the energy delivery.

Any of the devices or components herein may comprise a position feedback mechanism to determine the position of the device or components inside the anatomy. In one embodiment, the position feedback mechanism is a means for introducing a dye (e.g. a visually detectable dye) or a contrast agent (e.g. a radiopaque or ultrasound contrast agent) inside the anatomy. In another embodiment, the position feedback mechanism comprises one or more electrodes. In another embodiment, the position feedback mechanism comprises one or more distance markers located on an outer surface of the device or component. In one such embodiment, a medical component 110 introduceable through an arm 102 comprises one or more distance markers on the proximal region of medical component 110. In clinical use, the distance markers lie outside the body of the patient. The relative distance between the distance markers and a proximal portion of arm 102 enables the user to determine the relative distance between the distal ends of arm 102 and medical component 110. This in turn may be used to determine the degree of deployment of the distal region of medical component 110 relative to arm 102.

Any of the devices and methods disclosed herein may be used for electrophysiological isolation of the pulmonary veins (PVs) to treat atrial fibrillation. This may be done by creating one or more lesions around the pulmonary vein ostia. In one such embodiment, an anchoring mechanism is used to reversibly anchor to a region inside or around the pulmonary vein ostia. Thereafter, a medical system 100 mechanically coupled to the anchoring mechanism is used to create a series of lesions around one or more pulmonary vein ostia. The anchoring mechanism may be one or more of: anchoring balloons that may be anchored inside the pulmonary veins, guidewires that are inserted inside the pulmonary veins, magnetic devices, devices comprising a means for applying a vacuum, etc. The medical system 100 may be pivoted around such an anchoring mechanism Similar anchoring embodiments may be used to perform other procedures disclosed herein.

Several embodiments of user interfaces may be designed to manipulate one or more regions of medical system 100 in the anatomy. In one embodiment, the user interface is a haptic system. The haptic system may have one or more of: force-feedback ability, ability to apply vibrations to the user and ability to apply one or more motions to the user. In another embodiment, the user interface comprises a joystick. The joystick may have a force feedback capability. The joy stick may be configured to have one, two, three or more planes of motion. In a particular embodiment, the planes of motion of the joystick correspond to the planes of steering of one of arms 102. In one embodiment, medical system 100 comprises 2 joysticks—each joystick controlling the movement of the distal portion of an arm 102. The joystick(s) may be mounted on a console. The console may contain additional features such as displays for fluoroscopy, displays for pre-procedure imaging results, results of a real-time diagnostic modality, displays of the medical system 100 position, etc.

In one embodiment, the user interface is similar to the Sensei™ System made by Hansen Medical, Mountain View, Calif. In this embodiment, the user interface of medical system 100 comprises one or two robotic controllers along with several displays. The displays may display one or more of the following: fluoroscopic images, EKG data, X-ray images, intra-cardiac ultrasound images, external ultrasound images, pre-procedure electrophysiology maps, pre-procedure anatomical maps created from pre-procedure imaging, etc. Such a medical system 100 can be designed to introduce a user selected catheter into the anatomy as disclosed elsewhere in this document. The user selected catheter may be a catheter or device for accessing, diagnosing or treating the heart. Examples of such catheters include, but are not limited to ablation catheters, mapping catheters, pacing catheters, ultrasound imaging catheters (ICE catheters), guidewires, sheaths, needles, prosthetic valve delivery catheters, pacing leads and angioplasty catheters.

In another embodiment, the user interface is similar to the da Vinci® Surgical System made by Intuitive Surgical, Sunnyvale, Calif. In this embodiment, the user interface of medical system 100 comprises one, two or more robotic controllers along with several displays. The displays may display one or more of the following: endoscopic images, fluoroscopic images, EKG data, X-ray images, intra-cardiac ultrasound images, external ultrasound images, pre-procedure electrophysiology maps, pre-procedure anatomical maps created from pre-procedure imaging, etc. Such a medical system 100 comprising a robotic user interface can be used to treat a variety of internal organs including, but not limited to stomach, gall bladder, colon, rectum, urinary bladder, uterus and other regions of the female reproductive tract, regions of the male reproductive tract, esophagus, heart, lungs, liver, spleen, small intestine, and other pleural, visceral or peritoneal organs. The procedures may be performed on the surface of such organs, inside lumens or cavities of such organs or within an interior portion of such organs.

Any of the intra-cardiac or intra-vascular procedures disclosed herein may be performed in conjunction with an accessory device placed inside the esophagus or on the external surface of the heart or the pericardium. Such accessory devices may be selected from the group including, but not limited to imaging devices such as Intracardiac Echocardiography (ICE) probes, temperature sensing devices, shields for shielding microwave energy and components of a surgical navigation system.

MRI imaging may be used along with any of the devices disclosed herein to visualize one or both of: portions of the devices disclosed herein and portions of the anatomy. One of more components of the devices disclosed herein may be made of non-ferrous or non-ferromagnetic materials.

It is important to note that the embodiments shown herein may be designed to be used in the left atrium of the heart and within blood vessels to perform one or more diagnostic procedures (e.g. electrophysiology mapping procedures), one or more treatments (e.g. ablation procedures) and combinations thereof. In such designs, one of arms 102 may be translated relative to the other of arms 102. This allows the user to change the relative positions of regions of arms 102. Also, in such designs, one or more arms 102 may have multiplanar steering ability. The multiplanar steering may be used to deflect or steer the distal region of arms 102 in multiple planes without torquing arms 102. Arms 102 may be introduced in the heart using an auxiliary device loop such that each arm 102 is introduced through a single femoral puncture. Thereafter, auxiliary device loop may be exchanged for medical component 110 to create the looped medical system 100 inside the body. Embodiments of medical system 100 comprising arms 102 and medical component 110 may be sufficiently flexible and have a sufficiently low profile to enable a user to introduce the medical system 100 through the vasculature. For example, medical system 100 may be introduced through the femoral vein into the right atrium and thereafter through a trans-septal puncture into the left atrium. Medical system 100 can be used to treat various tissue regions of the heart including, but not limited to right atrium, left atrium, right ventricles, left ventricles, ostia of pulmonary veins and other vasculature connected to the heart and valves. In embodiments where medical component 110 is the only device between two arms 102 in the heart, medical component 110 may comprise an electrical working element. Examples of such electrical working elements include, but are not limited to linear or non-linear ablation electrode(s) or antenna(s), linear or non-linear mapping electrode(s) or antenna(s), linear or non-linear pacing electrode(s) or antenna(s) and linear or non-linear electrosurgical cutting elements. In embodiments where medical component 110 is used to introduce second medical component 110 between two arms 102 in the heart, second medical component may comprise an electrical working element. Examples of such electrical working elements include, but are not limited to linear or non-linear ablation electrode(s) or antenna(s), linear or non-linear mapping electrode(s) or antenna(s), linear or non-linear pacing electrode(s) or antenna(s) and linear or non-linear electrosurgical cutting elements. In the embodiments where electrical working elements are used for ablation, mapping or pacing, physical motion of a component of medical system 100 may not be necessary for the medical action. For example, medical system 100 may be used to position an ablation antenna of medical component 110 adjacent to a target tissue in the heart and thereafter ablate the target tissue without moving any of arms 102 and medical component 110. The region of medical system 100 extending between the distal regions of arms 102 may or may not have a uniform cross sectional profile throughout the region of medical system 100 extending between the distal regions of arms 102.

In one embodiment, medical component 110 or one or more devices introduced by medical component 110 are magnetically enabled devices capable of being magnetically navigated in the anatomy. Such magnetic navigation may be used to further aid the precise positioning and orientation of medical component 110 or one or more devices introduced by medical component 110. In one embodiment, the distal region of medical component 110 such as an arm 102 or one or more devices introduced by medical component 110 are capable of being magnetically navigated by a magnetic navigation system such as the Stereotaxis Magnetic Navigation System made by Stereotaxis Inc., St. Louis, Mo. One or more movements of such devices can be computer controlled.

One or more arms 102 disclosed herein may be controlled robotically by a user. For example, one or more arms 102 may be Artisan™ Control Catheters controlled by the Sensei™ Robotic Catheter System made by Hansen Medical, Mountain View, Calif.

Any of the steering mechanisms disclosed herein may be designed such that a distal end of the steered device replicates a user motion applied to the proximal region of the device. In another embodiment, any of the steering mechanisms herein may be designed such that a distal end of the steered device replicates the mirror image of a user motion applied to the proximal region of the device.

Several examples or embodiments of the invention have been discussed herein, but various modifications, additions and deletions may be made to those examples and embodiments without departing from the intended spirit and scope of the invention. Thus, any element, component, method step or attribute of one method or device embodiment may be incorporated into or used for another method or device embodiment, unless to do so would render the resulting method or device embodiment unsuitable for its intended use. If the various steps of a method are disclosed in a particular order, the various steps may be carried out in any other order unless doing so would render the method embodiment unsuitable for its intended use. Various reasonable modifications, additions and deletions of the described examples or embodiments are to be considered equivalents of the described examples or embodiments.

1. A method for treating hypertension and other medical conditions comprising the steps of:
introducing an elongate device in a renal artery lumen, wherein the device comprises a transmission line and an antenna comprising a flexible radiating element coupled to the transmission line,
causing a change in the shape of the radiating element, wherein the change in shape is based on an anatomical dimension,
delivering energy to a renal artery wall, and
obtaining a feedback of the energy delivery.

2. The method of claim 1, wherein the antenna has one of: a linear shape, a planar shape, and a three dimensional shape, and the method further comprises the step of changing the shape of the antenna.

3. The method of claim 1, wherein the radiating element has of: a linear shape, a planar shape, and a three dimensional shape, and the method further comprises the step of changing the shape of the radiating element.

4. The method of claim 1, wherein the antenna further comprises a shaping element that is connected to a shielding element of the transmission line and wherein a microwave field emitted by the radiating element couples to the shaping element.

5. The method of claim 1, wherein the step of obtaining feedback comprises obtaining a measurement of one of: voltage, current, impedance, temperature, pressure, one or more parameters of returned power, light, a chemical change, a change in the imaging characteristics, a change in the mechanical properties of tissue, and deformation.

6. The method of claim 1 wherein the step of delivering energy to a renal artery wall creates a first lesion in the renal artery wall.

7. The method of claim 6, wherein a second lesion is created in the renal artery wall that overlaps with the first lesion.

8. The method of claim 6, wherein a second lesion is created in the renal artery wall that does not overlap with the first lesion.

9. The method of claim 1, further comprising the step of cooling the surface of the renal artery wall.

10. The method of claim 1, further comprising the step of using a microwave shield or reflector to change the microwave field emitted by the antenna.

11. The method of claim 1, wherein the device further comprises a tether, and the method further comprises the step of manipulating the tether.

12. The method of claim 1, wherein the device is introduced through a sheath.

13. The method of claim 1, where causing the change in the shape of the radiating element comprises positioning the radiating element against the renal artery wall.

14. The method of claim 1, where causing the change in the shape of the radiating element comprises expanding the radiating element within the renal artery and against the renal artery wall.
